SEQUENCE LISTING (92 sequences)

SEQ ID NO: 1 - DNA, 128312 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaaggtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccagattcgctttgtggtggaactcgtgtggcctttatctttatttctggtcttgatctggttaaggaatgccaacccactctacagccatcatgaatgtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaaggccatttccccaacaaggcgatgccctcagcaggaatgctgccgtggctccaggggatcttctgcaatgtgaacaatccctgttttcaaagccccaccccaggagaatctcctggaattgtgtcaaactataacaactccatgtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacagcttggcaagggtatatcgagattttcaagaactcctcatgaatgcaccagagagccagcaccttggccgtatttggacagagctacacatcttgtcccaattcatggacaccctccggactcacccggagagaattgcaggtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtaggaagaggaatacgaataagggatatcttgaaagatgaagaaacactgacactatttctcattaaaaacatcggcctgtctgactcagtggtctaccttctgatcaactctcaagtccgtccagagcaggtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacagcttcccacactcctagacagccgttctcaaggtatcaatctgagatcttggggaggaatattatctgatatgtcaccaagaattcaagaggtgagtagcctgatggtagtaattataatttcattatgtctttccaccatttaccccacttatgtcaaataatttaattgtatttcaaacctgttcaaggaaaagtacatttgatctttccatctagcaatttcaaagcacctgttcacatcccaaattatctgtgctcttaagtaagaggcagaaagaaaggaaccacccttctgatttcacatcaaaaaagaaatgccactggcaataagcaacttgcctggtgtggcataaatcatcagaagacttacagttgaatctaagtcttttcagtactgaggtggttcattattctgttacagtcttaaaattcacataaatatatactgccaataataatagcatacacctttatagcttacaggcactcttcttctaagtgttttacctatgttggcttatttcatcataaagaaaacaatggacttttgtgttgttttgtaaaaagatgcgcacattttaattaacatctgattgcacaagtctcctcccatatagaaatggattcttccacgcaatagataagaggtgctggggatatgatgatgaacacacagatttggtcatgaccctgtgggaaagagagatgggaaaaaaacaattctcttcaagtgtgatgagtgttacgaaagggagggaaaagttgaaacaggtttttttccaaacttttctccctccattattcgcagctgacttgggctccaccaacctggaaaactgcatggttggaatctgtctttataaaacgcatctcaacctgggccgagtatgcacactgatgtgggaaagttagagaagagcccattgtactaatgctcacctgctacagtgggagtctctgttaaacagtcttttcttcatagcattaaaaaaatttatatcactacaataaggttgaaattgatagagaatgtacaaacaatccccaaagtatatcaacactcttagttctgagtagaagttccagaaggcttcttgactgtctagatagcaagtctaatcatttgtgaactaagttaaagcagaaggcccagtttatatgaattggtattacaccatttgacctgagaacagccccttcatctctgagtgctttgactaaatgagcaacataataatagtaataaccccttacaagatgtcataagactcactgttgttgaagcaatttgagattttgactttattgaagcatagatggtgattataggcatgactcactgtgtggattctccctgggctcatcagtttcagagggcaagtgttggcatgtggacaaagagagggatgacacgtaaacatggcttattgcaatggggaaatattttcagtctcactgattgaatcctaatggttttataaattccccagtaccactgaaagcaaagcaagtaatcaggtgtgttttaggaataaaagcagcattattttaatttcgtattttcccctaaagcaaagccaaatggcattatgggagccaagctactggcagctccaccagccttctcctgagttctcggcattacagatctaccctcaaaggatgaggccagcaagcaccacagggtgcccacatggagaagagaaggccaccaacctcctcttagctggcacagaattgaaaaagtgtttttccaggaatggatacttcatctgttctgtatttgctagaattttaaaacgcacacacagacacacacaggcgtgcacacacacacgcacacacacacgagaaaaccacaaaccacacatttcaaggaaatggaagaattcattggtaaaattaagctaataagattattttccaaatataagaaactaaattttagactatttagccaaagaaatttgctctgatcttgcttttctacaacagaatcattccccaatcattttatttccctctttttctccccagtatccccatcttggtgggacaacagaacccaagaactggcttaacagtaaaatattttctgcatttgcccaaggacacattcccaacgaattcaaataaaggagactagaagaagagaggctatactacagtgctctaggggtcactctgtgatttgttgttgttgttgttgttgttttgagacggagtattgctcagtcgcccaggctggagtgcagtggcacgatgtctactcactgtaagctctgccccccaggttcacgccattctcctgcctcagcctcccgaatagctgggagtacaggggcccgccaccatgtccggctaatttttttgtatttttaatagagacggggtttcaccatgttcgccaggatggtctcgatctcctgacctcgtgatccgcccgcctcggcctcccaaagtgctaggattacaggcatgagccactgcgcccggccactctgtgattttctttaaggctcatcctagtattctcctagtccctaagtagatggcagtaggttttgttttttgtttttcgcagctggattaaggattgctgagaatatatggatgttttcttttaaatgtggaagtcaaaccaaacgttggagcattggcctcacagcagattatgactctagctgccttaaaataacctgaagactttgccttgccctagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaagtaagttttctgagtcctgcttataaattggcctctcatgttggttaagttgatggtttaacacttctaggtgaaaccaaacctggggttgcatctgtcttgtcttgctgagtggccttaggtaaagagacttctcccagaaagtccacttccccttgcagaaagggggcattgcttataagcaattctggacatgaaccacagaaagaactgaggcccacttggaaagggaacagaggggccatttcccactgatgtaattgaactagggctaagttcaagaggaagagaatgatccgcaaggaagcaacccagagttccaggtgaagctcaggtcagaagggccctggcaagtaaacacggctgtgggatgcttttacaaacacaatatcgtgaaaatctatgtgtgtagtactgaattacattccaaatggcaaattcctggcaaatcatcttccccacctttcactatttttttttttttggtcttctatggggtaaaggaggatggggtggggaagaaatgtaactggctgcccctctagttaaaaactgaaaagaggcagcaagggacatgccaaaagtagttggactctaagatagctacacacaacaaagcagctaagcagctaattgaagggaaattactgaggctcaagctgagattccaagcgggggccttgtttggcctctcagtccctttcatctgagaaaggcctcagttcctagcagtaatcagaggcaggcttctcagcctccttctcctaaagcagaataaaccacagggcaagtcgcatcctttgtttctctgatgaggccattactgagagtcactgtggcattttgctactaatgatgagcttgttattggtggggtacagcctattaatttaggttattcatcaaatcctccagcatggagttgaatgagacatgtgatgtggatacactaatgactatattgagttacaagcaatggggagtttctgtaaaatctgtcccttgtctcctggcagcatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgtaagatcccagctgggcttgccttgtgtaccctggacctcccagaagtgtgtgtgtgtgtgtgtgtgtgtgagagagatgtgccttcctggtagcacatctcatgtttgttttttgctaagtggactcttgcgtttcctcccccatccacagtcatcactggaatgctttgcttcagtgcccctgcctgggccctcccctctctactgcagcctacaatgaggttttctttcccattgcttgaattatatccctaatggaagggttcacaattctctgaatcctggctactcagataaagacagggaggaagggaggaagggtattttctcccagggggtccaaatctagctttaacgagggaggttctgagaaaataatatcatcaatattacatggacttctgagatactaagaaattagattctgtcagcccaggaagttgggagatggtgaattgttctgggaaatagcaatagactgagaaaataaaaacacttccttgaaaagcctttccctaacactaagtgataggggcagaaaagacacaaccaaaagttctctctcacttttctctctgttcgtgtctctgtcttgatctctgtctggttttaggccaactcaacttttgaagaactggaacacgttaggaagttggtcaaagcctgggaagaagtagggccccagatctggtacttctttgacaacagcacacagatgaacatgatcagagtaaggggggttggaggatggggaggggaggggaggaggaagcggtgggggcaagaaagttccacttgtttccttttcccaggaaagagttaatcgctattggagttagatcaaaatacaacaagcaggccccaaaggccttcattccaagcagtcaccaagtggggtcactgactttggatgagaaatatgtttcttgaattctgggagaagtctaaaagctgccacaagaccagtggcttcctggagtttcctacttttatgaattcactcaagggcctcaaattcaaagaggcatctccccaaggggccagctctgtaactccaaagatggtggaatgtgtttgtctggtctcattttcagctttgcaaaatgaagacaagagttctatatatcagggacactcaaaagaaaacaaaaatatccataagcaaaagaaagctttttatacaccatattcaatgacccccatctggcccctcctttgcccctacacatcttccctctattctagagacccatggacttggggaaatgggatatagataggtatgtttcatagtggaacaagctcaccagctcttcagggagccttagcatctctatcctcaatcactaaaaattagaaatggctgaagaacaagaccaaagatcctatggaatttctaagcagagcagtgactgtatttcttcttcccaaggataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggaggtaaggggctgcaagccccacagtgggccccttgaagatagccccatgagtggggccagagctcccttagcaagtcaagtggtcttgaatttaagctttcattttccccactgaagaaacaagaatccctacatcccctgtacagttctcattctctaacagcttatccatacttaaaacttatctatgctgaaaacggtttcctcttcacatctcctacttctcatgctgggcacctcctcctgtagccccctttaagcatctgtgtctgtcctcaaccctcttctgtctgacattgcttgagtggccatctatggccagtgtcccctcaaccccacagtccattgcttgctggacactcctgccctcaagttctacaagcacatcagcctcaacatgtcccctccaaaaactgtatgttctccttgcccatagaacatatccttctcctatatttcctatcctaattaacgtcctcagcatttgcccgaattctcaagtgagggatttcagggtcatccctaattttccttcttcaccctccacacagtagctgtcacttactgagtgttactttatgccaagtactgtgccaactgcttttacacacatatgcttcatttaattctcacagctccatgaggcttgcaccattatcattgccaatttgcagatgagaagccagggcttaaagaggttaaataagatcccacgcatgaccattaagaggagcgaacaggatccagctctgggggtgcctgagttcagagcctgcctttctgatttctcttaccaagctttgtctcctctccctcctaaatatctctcaactctgcctcttgcattccaggctctctgaggactagaggccttgtcatctctgcgccagcccattccaagggcttccttcctggaatccagggtccagcctctgttggcccaggcatttctctacactggcaccagagttacattccgcacacctgcttacgttgctctctcacttaaaatcttaatgactcgacccccaaataacacaggtcccttccaaatctgtcctaccccaccttcccagcccttgctcaactctgctacctggccctttcacgcctacaggcattcccattccatgacctcttgggattctaccctttgcaaatgctgttttcattgcccatttattagagcgcttttggtcacaagctttttgcttaaccaaaaagaaagcatttattggtggacataaataatgaagttcaggaggatccaagagttggaagccaccatgagacccctgtgtccttccacctcacttttctactcgcctctgctcagcttcatctctggccaggccctctcctctgctgatgccctagctgcttacagcccttagcagtcatctatacaccaaaaatccctttcccatagcagaagcaatgctcctagagagtttccctgttggtctggctcctgtacccacccctgtgtactctgattgggaggcctgggtcagctgcccaccatggggccatttctatgagcaggattactgtgaagtggaggaagatgtttccccaaaagaagaaacacaaggtagaaaagtgtatgtccaccaatgcctgaaatgactgtccctttcctcatctgctgagcttctactcattcattctttgagactcagcactcagctcttaaatgtcacttctgctttgatggaggtttagtcattcactcctctgtgcttcctggccctctcttcacacctctctcagacccctctcccagatagattagagttggctgttgacatgtccatctctggctgggcagctaaactggagttatttagaatcagggagcacatgtcagtcattttcaaattctcaacctcatactcccagtaaatgactccatctaagggtggaccactcttgcccatgggccaggtctgggtctgtgtcatctagaactgttggaaggtaggggcttctgtgagcagtaggagagggaataaactcgagggccctcgggagcatgccctcttgtctcagacttgtgagtcctgaggataacaaactagtgaagaaaagcctcgttctatctgtcacctggtgctcttgaggactttctgttgccctggtgccaccacaattttccagagtgtgtgaccctcgctctccaaactctggaagtggcagccgaggctccccagtggcctttcagaaggtgccagtcatgacagcagcaccaaactgcaggcaactactaagcgatcaccaacttgtctgaagataagaatgaccttgaatgcattttataaaacaggattttttttttaatttttagattttctttctttattttaccttaagttctgggatacaagtgcagaatgtgtaggtttgttacataggtatatgtgtgccatggtggtttgctgcacttgtcaacccatcatctaggttttaagccccacatgcattagctatttgtcctaatgctctccctcgcctcgcccctaccccaccccaacaggctccggtgtgtgatgttcccctccctgtgtccatgtgttctcattgttcagcttccacttacaagtgagaacatgtggtgtttagttttctgttcctgtgttagtttgctgaggatgatggcttccagcttcttccatgtccctgcaaaggacatgatctcattcctttttatggctgcatagtattctatggtgtatatgtaccatattttccttatccagcctatcactgatgggcatttggattggttccatgtctttgcaattgtaaacatacatgtgcatgtatttttatagtagaatgatttatattcctttggttatatacccagtaatgggattgcctggtcaaattgtatttctggttctagatccttgaggaatcacactatcttccacaatggttgaactaatttacattcccaccaacagtgtaaaagccttcctatttctcaacagcctcaccagcatctattgtttcttgacattttaataatcaccattctgactggcatgagatgatagatacccatttgtcagatgggtagattacaaaaattttctctcattctgcaggttgcctgttcacgctaatgatagtttcttttgctgtgcagaagctctttagcctaattagatccatttttcaattttggcttttgttgcaattgcttttggtgttttagtcatgaagtctttgcccatgcgtatgtcctgagtggtattgcctaggctttcttctagttttcatgattttagattttacatttaagtctttaatccagcttgagttaatttttgtataaggtgtaaggaagggatccagtttaagttttctacatatggctagccagttttcccaacaccatttattaaatagggaatcctttccccattgcttgtgtgtgtcaggtttggcaaagatcaggtggttgtagatgtgtggtgctatttctgaagcctctgttctgttccattggtctatgtgtctgtttacaaaacagattcttaagcatcaacccagatcgactggctcagaatttccagggaagaggcctggttatctgcatgtttacagacctattagatttgtgggacctgcagttcccttgtacagttagttactcaattaacatctccctcctctcatggtgcctctacctgctaagcccttattcccagccaggcccaccaccatccacccactgctgttataacataagcaggacctgtgcgagggggtgtggacggaggagagaggctctgttgcttcatttgtgcagcatggagttcagtggttctcacaatgtttttgcaaagtatataaagaatactccttgtctacttgacattcgtatcgtgacataaatgtcttgttttccagaaggattattttttccaagcagcttgttcctaatgcagccccaggcaccaaacagatacttaaaatatattaattgcttaaatggttaagaattcagtctctggacccacactgcctgggttcaaattcctattatctgtgcccagtttccaagtctataaaatagggatattaatagcacttacctaataggctcgttatgagaattaaatgagctaattcatgcaaagcactgacatatagtaagcacttaataaatattagctttttaacaaaatacaagccaaaaaacactgcttaggagaggaaatgatgttagtgcctcctgtaaataggcccagcctccaagctggtgctcctctaggaatcacaacgctgcaaatcacatcctccggggccgccaggacttcacgagggcctctgagcagaggggtatgatgggagcagaagcccagcagctgtgatgatgtggtttctgatcttcctgcccttggggtgggggaggaggaaagcaaggggcaatgaacagaaaggagaagatagcggggaggaaatgtgtgaggaagaaacacatcactgtggcttgtcctggatttttctgcttctgttctcgtgttttgggaagtctggaggagacttgaaaatcattcatgtccccaccctgaggatggcttagtagcagagaggccatgaaaactctttgctgatggctctgaaagcaaggatgttgcttcactgggctgctgaaggcctgcctgggggttctgagcagagagtacaggcccctcccaggagggcggcctaaccaccatgctggcatttctgtggaccatggtctgctgtctcagaccccctccacaatagggtctgcaatctcattcaccccataaatacattctgtctttcctctgatcccctcccattagcagggggaaataaatggaagtcagacggcccagttagaaggcaggcagtggagtaggaaaatagatgatggtggtttggggagcctcacatcactcatggggagacattcattcccatgggccttccaatcacccttttctccaaatctaaggacacaggacaaatgggtcctcatacaggcaaatatcttaaactggtatgtgtattcatttatagttctaatttatatgtgtctttattcacatatattttgcttctggagaaaagctcaattagaaaaattaatacattattcttcttattgcccttcagctaaaacaagcatacacacccctcccctttggattttttgtttagcaaaaggttaggcctggcacagatgaaatactattcagagttcacagtgtattttcatttcataatatatttgattttcaggtcttgaatttcacatcaggaagctgatataggaagctgaattcagccagattttaatacgaaaatacctctgatcaaggcataaaattgtactttaaccagtaaccactgtatttctctaagctgtgaaaaaacatgcattcattaactgctttttcctctgctgtcaacacagtcaatacatgtgcataactccttattgtctacatggtgattatcttgctgatgaattctcaaaggccagagatttggactattttttctctgtaaccttgcatgttcctggccacatgccaccaccacccaaacagaatgtacgcagggaatgtatttttcaggataacctaagaaaaaataggattaagaagataaagctgctgatcatgtaatgtactttagactcagatatataaatatttgtgaattatctgtcctatttctttcttctattaattcattgactctagatgtgcattggaaggctagggagaaatcaggggatcgtgagaaagagcacagaagtctgcatcacacaaacaatattatttcaagagccatgaactagatcctaagcaactcataggcaatgacctcatttcatacctctagtctctaagaaacatataactggcctgaggaaggaaaatgtgggcaaggggtagaccggggtcatgggtggaggtccaaatagtaatcaatggagctcatagggtggactgatattgaagctgctatgagccagccacatgctgggcactgttacatgtcatctcatgcaatactcccaattacctgcctagtaagcataattgtcattttatagaattaaaaacagactcaaagaggttgacagtctaatgtaacacaacagctaaatgggggatctggaattataatccagagctgcctggctctgatgagaaagctctttctgctgtcatatgcagcccacattaatagggggctcagaaagtattctctggataaattatataatgaatccaatgaaggaagacattattttataatatgcagcataataggcactattatgattggattttcctgcttgaaagtagctagattagagtaggaaaccaaaaagatgtgaattcattcagtcattcatgcatttgcatggattgagctacctacatttgaataaatgctgttaatccctgattccttggaagctcacattggagagataagcatgtcattaaataatgccataatagtggtatctcagaggactagcagaacataattcaatctgacagagtagaaacagattgtacaaatccaattcaaaacatcataaatcctctaagcactgtcaattcttcctccaaattatctctgaaattcctccttctttcccatttatggcctccatttacagaagcgtgtactgtctctcttagctgtttgccaggccgccagtctcttgctgttcagctctcaactgcttccagcaagatctttctaaaatcccaggcttgccaagacttagcgcccacagctccacagtgactcctcattgctgttaaggtaaaggccttcccagtctagcccttcatgcttcttccatgttctatgggactgccccaggcttcccacctggtaccactgagcctttccatccttcccccactcgactgccaggtcaacacccacacccacgcttcaggactcaggtcctatgtttcgggccttcttctgtgcaccattcccttccctgtagcccttgatcatgatttgtttatacgcctccgcaccttcatggccctgaacccctcaagggccgaaactgccttacttttctttttgacttcccaacttaccttagtggagctgtagtcacatagaatagacgctcataaatgcttctctgggctgtaaaggttgaattttccagctaagcaaggaagaaagacaatttcaggcaggaggaagggcataagcaaagtgcagagatgtgaagctcaagagaaatggatgggctgggcagaggtgtggctgcagcatcaggggagaagaagtagtgcctggagtcagcaggcacggcttgcaaaagcttcacctataggtgaaaggacaccatctcttgcaccaataggctctgtgattggaggcaactttgctgttttactgccagaaaactgaggatgataacccaaactgcagttcaagtggcattcactggtgtggctgaaatgggtgtttgtggccagaatgtggtctgattggtcagtgcccagctctgttgattagcagatgttttgaatatagtagcatccatgtgcccaagttgttgggatgattcaacaagaaactttaagagctcaagtgccctgcagttgtcagccaggtgattctcttcctttggacccagttagacgcaggcattacctcgtggctttgccccagtgtgaatctttgtcctccaacttgatctttttatttgtttcattattgtatttaagttgtttattttagagacagacattttttaacagctgtgcatttcctgtccctttgttttccagtcgtcatgtgtttccttactctctgtgggtgaacgtttcagatgtctgtttgcggtgcccagcgtgcaagataaaatttattgcagtgccttcggcctctaactcaccattccaaccaattcagatagcccaaggctgttttatccagtggatttttccatgtagtgggaaataaatcttgaatgttactgtttagattagccaggaaactcattctgggatgtttgcccacatccattggcatttctcaaaaggaaccccaggtgtctaccttgacaccagcagggccacttgagccctccgctggcattcatcgcccgctttgttctcagcctgagtttaggagttacagatgtgagaggcgggattatacagccaacatctctaagcgggcagtggctcccttaccctcgaagacctcactcctagcacgtcctggatgtattcgtcaaaatatgtcctcttatgccacgtcagcacagggttgctccccactttgatcatcaagtttaaacaaaaggaaagattttctttctttctctgcctctactggacatcatttcccacctaacagataatttaatgtatctgttactgaatgtgtttgaattacagacagagaggtcacagttaaagaaggaagcctgctgctactgcagcttgtcctcccaaggaggtgtttgatttagctgtgtaaacaaatgactgcattctccagaggtcctgaacacagctgcctgcgctggagagggctcaaacctcttccgccagggtgaactctgcttcctggtgagtgccagcaaaacaaccaacaaagagctgtaggacttgtgtggacttcaaatggtggtggtcctgccacttgggctcagccacagcagttaggaaactaaaggggaggaggaaagccctttccttgctttattgtcattggctgtcatagggcattacaatggttctctttgagattctgagctccggctataacatttgcccagaatctgcctctgaggccttaagacactgtgtttttattcagcaaagatgccctttgactccttttcccactagtggtgctaggtttgagcaccttacactggccccttacaatagccagttcttgtctacctacattcttccctaacattcatgattgcatagttactcttagtgtagaagcagacagcttttacacatagactccatggccgtagcctcatagaacctactatattctaacttgcaagctaatcagaccaaatatatcaaaatcaaaaacctctgctgagagtttattcattcatctctgtctcccaaacgtacttatgtacatacgtgcactaatatacatgtccattagccaagattttgatttcagggatcaaagcaagtaccaatagggaatgaggtcacttgctgcatggcaggtggcttccccatgagaatgcaaggccacctcatgactcatacttcagagggtgacccaggaacttctgattcatgtccaaagcagcttctacaattgctctaccttgatctagggaagatgtggggaggatgacattcgggattagctttataaggccttcctgtgggcagagttgtctgactttcacctagtgatcaacaagcagctagcaagcatcagtgtgtgaggccccacgccctctcagctcccctactgcccacctgggacatgggctttggcatctgtccatagcattgttctaaccaaatgaggtgttatggatcagctcaggatgggatatgttcccagacatattatttaaagaaaatagctcccttcctcccctgataaacagctgccatggctaaaaggtaacctggctggggcttaaaagtctgttgactttcaagatattttgcaaaaacagtcataaaaatggtatttatcagatcctaactatttgtgagacggtttggtataccatagtggttaaaaacacaggctctttccagaggaggtttactttgcttagtcgtgtctcctaagtgaacttggacctcataaggttgttgtgagaatgaaatgggtgaatatgagtaaagtccttggaccagttttggccgtatagtaagccttcagcaagcatctgcttttattcctacagggaggcaattgtaagcccttcacaaacagcgtctaatgtgatccttagaacaaacctatgagatagggcatatctcaattttgtaggtagggaaacagaagccacacaattaggaaatggcaacagatctgttagactcttaaacactatgctacaccaatttgcaaggcaaggaagacaaagcacctttgaaaatgggtcagatgttttagggtaaatgaacgtttgagaatcttttaagttttttttcccccagagattatcaaggtatcattgtagggggatgcatcaggaaacatgactatgaatcagctgcctgataaaccagccaggatggagcccacgtcatcacagcagtcagcaatgccactgaaaaacatcagctgcttattcccgtatagatttccccttaagacatgaaaagggagttcaaagagaatgggccagatatctctgagagtcatattactaaaatatatttatttttactagcttttttgttttaagaggtatactgtcattagcactgtagcaaaaattcacgttttattaatttctcctagtttatcatgtgattctagggtaggatgcagagttatattcaaaatacacaaatcaactcaactcagtaaacatatatcgaggccctatcatgacaaaatgctattctagagaccacggcgaacaagccacggccccagcctcaaagaatgtactatctttggaactgtgctggccaatacagtaaccagcagccacgcagggctatttaaatttaaattaattaaaagtaaaaacacaatgcctcagatgcattagccacattttaagtgttcaatagatatttgtggctcctgcctgccatattggacagggcagatatagaacaattccatcactgcagaaagttctactgaacaatgctgctctggagcagaagatcttcttgttcagggatgttacacccccgcttgtggctagagtgtggcttatcctcagagcaaggataggggaaccatggcactctgcaggctcagcactgaagacacggatgcaggctctgcttctgacctagattgaccttgggcaaggccctttgctcctctgatcccaatttcttcaccagccaagtaagaacatcagaccacaagccctctagggctctgtccaaatgccccatgactgagtgaactggtagaacattctatgtgtgtgtcacaacatgaagagcaaagactttcatctccccaaataattttgtttttcgttttaggaattaaatttcagattcactctaattgccaatactaaaattctctatatgcagttctaaacttgacaaaccaataaaaaaagattatttgactacttatctttgtacaacattgaggtctccctaaagcaaatttaaatgcatattttaaaaatgtattctagcagttcagttcagaagccccctggcccaagcatcacactgtcaatcctttgtcctcaagcagcatggttgggtgggttaagtactgacaaacactgggtgtcaggcccatggtcagggactgtgctaacagtctacatattagatgccacctacccccaccctcaacagacccaaactatttatccaatagcaaaccttgcattatttctgtccagaagaaacaaacatttattgacaacttttggtgtgtgacctgtttaagtcctacatctcatttaaggactggtcaatgttaggctaggcaatgcctgtttgtgagagaatcactgcctaaagaaaattctccatttcccttagctctatggtgggtgactacacatactggtatttcttaaagaaataccaattccatttccttttaacataattattaatatctcattagcatggtgtcactgaagcctgggcccaaagaaataccaattccatatcattttaagatcattattaatatctcatcagcgtggtgtcacttaagcctgggccctttagaatttttcatgtacctgtgttcctctgcccatatcagctggaacactaatagttttcttcctttttatctagaagactgagaacattacatgggacctgcccccagggcatggaggctgaggtgggacagtttagttcaggaggcccaagaagtgttgggtgtgcagccccttgttcaaacacagcctctgaatcgccagaggcttccggtgcatactctgaggcgcaggtgggactcgggagtgagaggtttcggcgaatgaattgggattgcctacttcttcccagtgcagtggagcttggttctgtggtcaggtccttacgccctgtctgcctttctcgtttctttatttctcgggtagtagttgtggaatcaaatgacctggggtttgatacctactctaccacgcctctgggggagtcactcagactcgttgaacctaagttccggggctgccaagtgaggataagtagtaattgctgatccacctacttgacaagatagtagtgagggccctgagcgccaggctgtggatccagcctttcccacggttcctggtgtggcaggaagaactctaggcctgaaggtgaaattggggagggagtcccagctctgccactgtctctctgggtgacctcaggcaggtctcctcaaaaaaataagatactttataaagctcagtttcctcttcagtaaaatgaggattccaggtaactcacagatagtttgtggggatgaatctgttccttaaagcctgcagtacatcaataacccagtcttcctgcttgctttcccccctctccactaccagtgatcatagtctgatcccataggtgatatcccagctcaaaaccctacattagcttctgtggctgtttaaggcctgcccagaactcccctggtcttagcactgaaagcacgtgtccggggaagccctgcattggtcgttcatactactgagtcccgcagggcaaaccgtccggtcccaccctcctttctagtgctgctgtcacactcacctcccttcaccctacactcccttctgtgccttgcaattacctagggagttttttacaagatatggatgccctggccctgccactagagattctgatttaattgcttggggtagggcctggcataggtatcttttaaagctccgcagtggttctaaagcacagccacagatgggaaccactgatctattcttgtaggtccccagatacctcatgtgctgttccctgtgcctgagctgacctttcccccactttcctctcctcggctaattcctgcttatcctcctactcaggaggctcttcctccaggcagccttccctgatccctccaggaagacttagctgcgtccctccgctgggcttccccaatacactgggcttgctttcattagaacctgatccttccacattatggttgttggtttgctccaatcctctccctcattagctctcaactttctttcaggaagagatgtttatctttccttcttgtattcctagagtcgaccaggctctggcacattgcagattctcagtatgcattcagggaacaacttaatcaagacaagaccatctgacttcttgtgagttacatgctaagaaagaaatgtcgacaccaatagccctcacaatgataggaacaggaggttaaagaaaaggaaatagatgcaaatagcaatataagtgctttaacaaatctatacaggaggacaaccatcatattcaaattttcaaacattcttagttctgctcttttgtgggtaatggtttttttttttcctcttccaggagaagaaaagaggcatattatagaaattcctcctcccccagcattacttgtcacagaattgtaattggaagtgatttccctgactaagttattttggctgtctgttattttctctcttcctccttgctcttccctcagctggccatcctgtgtgtttggagagagccagaaaggttcaaggctaggaatgtttctctctctctttaaagctctttaatcgtcaggctttctgatcttcaaagcaggctgtagccagtgtgaccccactccctcgcctccccatgctggagagtaaaagcctggagtatttttgtcattttgaagacttgcatatttggacagccttggacatctggaaagtgtggtcctcactagctctgcagggataagagcacgtcagcacttccaagctctctggcgcccctacatctggacacgttgaaaaattaacaccagactctggagttaagcaaacattaagtttataggcctccttgcatttgaccatttcctgggacagcagcccttatcctgtgactttctgtgtgtagagttgagtctttgcagttggtcctcctcacactctctcaactttgtgactctctgcagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtgatgtttcaggaagggctcgctgcatttctccaaagtcagtgggaaattacatttggtagagagaaagggattgagactggactcataaatcaataaaattaagttaaataagaaaaaataagatattttataaagctcaacaaagagtccttgaatgaaagcaattacagagtcacattgtggctaatattcaaaactgagatttaaactgaggactaggaaatagaattggatccttttgaagcgtttaggagaaagattttaagagaatgagttccgagtcaccctgtggtcgggaggtgtgagtgagctatccaagcccgttcccatcctttgtccctctgtgtcttctcaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattcgtgagtctgaagttcgcgatcctcctccatgacacgctaatgggggtgctggagtgggctggggtgggctgggggtgccctcaaggcttccatgtctttagagagagccccagggaccagagccaaattggagagcatggagctctgactgaggaacctgcttctcccaagctccaggcaggcacagatgagtcagtgcagtggtgggaaagggaaaagagttgatgttgtagctggaaaagggaaggggaaaattaaagcaaggaaagtgaggctgggggaggggacaaattccccactatgtagtatgtttggtatgtggaagggttctggtcagaatgtttgcccaatgattgccacatcagcattcattttggactctgtatggccagtaggtctggttcctgggagccctggaataatgcagccccttccctaactaacatttccatgatgtatgctcaatgacaaggcagaggaatgtgttggatgagctcaggacctgcctccctggacactcccatcccaggcctgtatatctgttgaccaggaataagccaagcaagcagcctactgtttgactgaatatggatttggggggtggtagagaaagggccggggtggagggttgggaggctcatttgtcattatagatggggtcagacacactaccaaaacagcagcagagatctacaattgagttcacctaaaactcagtgtggacacaggaaaccctcttttaataactgtccaatgggttttccagcctcagctctacagaaaacttgagataacagtggccagtctgcagttagtttgggttcggacaataggcagagctgggaaatggagccaggggcgaaagcccaggtccactttaggatcaggacgggagtggctggtggggaagtgaggtgggtgtggggaggcaatagggagctgggtcatttggtatgggagagtcctctggtggctagtcccagaagtgcatgctttacgaacatatgcttctctccctagggccaccttgagtgaaaccctcccatgctggaattgggccctttcagtgacaacacacaacagttttcaatagataataatcccaagggctttactagcacatgaaacacagggaaaacgtgtaaagttcacaagaaagtcgttccagtgtatcaaatctatcctgtttgccaggtggatataccagggtctcctccacctgtgcatggctggtggtgggtccagtggctgttggataactgatgtattgatggatcattcgccttctgaaagtgccaaactgattagttattttgtgtgtctttttgtgtaactagggtttgaccttccagggcagactgtgctggggcggctgaccccttggggagccaagttattgctcttaccaccaccacttgcccttgtcagtcctccaccctcttgggtttcagtgtcagcatgtagctgtctactcagatcccatccacatcatcaagtctgcagttttttccttgcaaggccttacagggaagatctttgacatagaggatataattttattgacacattttacttgcagagcattcacccgggctaaccagaaagccagcactctgctataaacaaaaaataatgcttcagggctaacatggaatgtgttaaaagattccagcccattaaatgtccaggggaggttttcctgttttcctttccctccatctgggctttgttctcaacacattcattcaacaaacatttattctgcctctaccaggtacagagcactctactattctgcttctctccttttgctttagtttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatggtaagccaaatggagaaggcccagaaaatcttgaatactttggttcctttcccctttcctcctgttcatgtgcctggattagtcatgtggccaccaaggagagcgtgacatctagcttcccagcccttccttttagccaacgtgggagacactcaaagagacgaaatctcctgaaggagccactgtatcacagcatcctcccatctcccacttcctgcccaggggtccatggtccacacagacttcccagtcccattccgtgaccatctggagaagctgctattagcagagccctgcacagggtgatagtgtaattaaagtggtcttctctttccaaacacagaaaaaatcagttcagggagtgttttcctgggcttacaattttaactactggctagagttgaaatggggaaagccttttgccttttcagtagcagtaggggaggagatctggattatttacttatcatcatcatggtcacctcctacatggcttcaccaaaaaacattctgctgcctgaaaaagctccaacacctctctctcttttaaaggatggaatttggagtccatccttcctcagtgataaggagtttttatagccacaggcagcatctattggtctgtcctctgcaaacttgcaactcctctgagagctagacttggaaatgaaacattattttgcaatgcgctgctatccttcatttttagctcctccaccgtagatgatagtttgtacttgttaaatgataaggatataaatttaggtcattttttatattttattgggtggaatttggtataatttttagacttcaggctttacaggctcctgagatggactgattgagcttgttctacttcttccccatcatgataggaagtgctgtaccacactaggcagtgtgtgtagtgaccacagactggctgagtgtctcccatcccatgctggcccatatctggtacccacctgatccacaaatgttccatcagatcctgttcaaacaacacatctccagttaagccaaatcttgccctttctccttacggtaaaatgtactaaatctgaaggttttgtctttttaatgttgctccatgatccagtgatctgtggccttggttatgctctgtgctagagtcctaacaagacaaatgctaaggtagaggtcattctgctcaaacaacctgaccccacctggatgtgggcttacatttgcaaagggcaccaaagttctaagagatgaggggaggagctgagccccttgtccttatctaggtttcccttgttctttcccatccctcagtctgcttcttttcccagtaccaacatgtttgtgtcctcagaattaaaggagtaaaaatgtgtaaacatctgactagcaacagccatgagattttgcctggcttgttgataagcagcattgagatctgccctcctaagaatgggccattaggtcttcaaagcttttacgatgtgaggtaaagaatgttcaccaggagtttcatgcacaaaagggtttctctttgtgggaactagaacattgttccagtgatgacggaaacagggctttccataccaaaacagggttttcctttgaatgactctcccacctttcccttgtctcttcctccccacctcaacaacacaggaaagaagctggaagcagggacaatgggaaggtccctttgttactcgagctattagaaacaaaaagaaaagtggccatctgaggaagccacagctggtgaaactgtagggtcacagagtgaattacacctctggcttaagtcagtgaaaagtcctagaagtttgtggtcctagaagtcctaaaagtttatgggactttgttttgagcaaggataagaaattgatttcaggctgggcgtggtggctcacgcctgtaaccctaatactttgggagacagaggcaggtggatcacttcaggtcaggagttccagagcagtctggccaacatggcgaaaccctgcctctcctaaaaatacaaaaattagccaggtgcggtggcacatgcctgtagtcccggctactcaggagactgagcaaggagaatcccttgaacccaggaggtggaggtctcagtgagctgatatcatatcactgcactctagcctgggcaacagagcaagactctgtctaaaaaaataaataaataaaaaagaaattgatttcattcttctgagaactgcaacaactaccttaaagtgattccatccaaaacccacatgttcagccatggacttgcttttatggagctgcgtgtgggtgacacacaaaatcaggagctctgagtcctaatttagacttttatttagatttcctcaaatttgggttccagttaagcgtgggtctcttctgtgccccgctcccctttgccatttgttttatctgttcttcagtctgttctgtcagtacccacaggcaggagagcagaaaggagaaatggcagccacagcagacaaatggcacattcgttccactcagctctcgcatgcccatcacagatacagctcattggtctcttttctatgagaggaagccagagctccagggaactactgccaactgatcagaactcatttaggacatggacctatttgttcctttatgttcctgggaagagcacaggatgaattctatgtactcatttacgtgttcagagagtaaagtgcctcataggatgcctccagcaaaagataaccaagaaggtctaatacctttgacaatctcagtttatcctatagtgtaattggatagcagttcccctagcaaaagttgctagtttggtcctattttctacatagccaaagtgattgattcattggttaatgtgaaagttactgagtactgccagcaggttctaggaaatatatttgtgtgatattcatggatggggaggatcaatccacttccaagtgatttggattaattactggtattttcacctgtgtgggtagcaaacctcagaaaatcaagtatagatgacggcataggacaggccaggccccaggcaaaatgttgaagctcctctggagttccctcccatctccctcttttgttttccatatacctggtttatccagggccctggagatgctccaagaccccctacccaggtcttcctcccttgtcccagctatatttctccatattaccactcttctcaccgaggatttgcttacttaacacataataaatactattaaaagagaaacttaggcacattaaaatgttagagttgattccagcaaacagtgattcacaggaggctccagatcacaagtggttcagggccccactgaggggtagggaagcaagacaaagaaaaacaaagcaaatatttgattggttcaagtggaaagtccctgattacaggttagtgggcagtttgtgattagttaagtttctctaagttgggttttggtttgctgatgtaggaacacagaatgctggggccgtttcaacctaatggtctcccaattaatttttttaacattactgatgactgttaggagtctaatgtgctactcctcccagggaaaatggcattcctaggattaaaggaactcagcacatggagtgtgcgtagaaatttagacactaactgcaggctggtgggagagagccctttagggcagaatgagaaggcgtccggccaagggcaggagttactgacgcatggcctcttggtttcagcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtggtgaggcccttgggctggcccctgtcctacaacacgtttccttggaagggtccgtagcagtcctggaggcccagcctgccctctgagggggtccactttgcctttgacctaaggttaaaaagttcacgtgaggctaaaatgtacaggggcaaaagtgggagcagtcctcaccccgagcgatgcaacagtgactcctcaccacgcctgcttgattcatctgccctggaaagtcattaaaaaaccagttcaactcatgggtccctttatttactcacaagagagagccagcagcccatttcactagttttcctttcctactctttgagaagaatcagaagggagggagcttgccactttactatctgtctaaagagatgtttccattaattaaaggtttttgttttgcttcaaaaaaacttgaattggagtatttccacaagtatctttaacatgctctaccaatgtttgcagaaagaagtgcagaaatgagactgtccacagagtcaggctcgctggccaggagaggactcccgaagctgacttctgatggcctgagaaacttcctagttcacaattcccagacccagacaaagagcactgtcttttctctaattgttttcaaatgggccatttccaccctctaatcagcctctggccctggagggtgcagttccccttgtcctccggagtctccctgtctctgtgctgtagagtcaagaagggacaaccacctgccctcactgggaaaagacagaaagtctgacttgttctcacgactcacacttattaggctccagaggtgtcagggcatctgcctttcatttcttaggttaaataagaaatcaattgctgccatttgtagtacccaattttctaaaatgatcacaatggataagtggcaagaaatccttatgactcatctgtgggcagagttgggctattttggtaatccttgagtaggcagatggaatttgaggccatcttcttgggtacatagatcactaggaagctataggtctagcaactgtggattagggctgggctgagaattgtttcatgttttttgtgactgtatagctagagactctcttgtttgcagagagacactctgaactccccctggccgtcaagggaaagactgccttcaccctcctgagctgaccttacactgagagacaatggggaccctcttttggccctcccctctacctcgagggcatctgggtgctgttgcattggataaaaggcactgctctttttctgtgccctctccgcctcactgcagagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggtaagcatcctcctctatagggtaaaggtaattgagttcttcagatccccagccctctccattcatctagtttaaatttcatttcttccaagctctttgtcagaaccagcatttgaagtttaaatctagaagttaaaaatccaccagcaaatcctactggctctacttgagaaacaaatccagaatctgatctcttgtcaccacctccaccacaaccttcccaatgccagtctcttccttccactaccacctcccatcagtccattctgcacactgtattcagggagatcctttcagaatcaaggtcatgtggtgtcagccctctctgtcaaatgcttgcactggcttttcctctctttcagagtaaaacccagtgtctcaaccctggcctccaagctgcttcattatccggcctccaactctcttcttcatcttacgattttccctactcctccatgttcctctgctccagccacgtcggcctccttactgactgtttaatacaccgagcgcatttcctcttcagggcctttccacctgctgttctcatgccagaagcacatttctctccccacaacctgcaacccgcccctcatatctgcaggcttgcttccttactttgttaaggtctctgttcaaatgtcccattatcacagggatctttccagactgaagagatctacataactatggctctgtaaacaacattcctccagggttcctgtccccttaccctactttattttggggaacattcttcaccatctgatacaatgatgtatcttatgcatgtatttactgactctctgcccttagtagaatatgagcccagagagcatgcatgtggtctattttgttaactgtgacagtcccagtgcccagaatagtgcctgacctttggtgggcactgaataaatatctaagtaatctgtagcatggaaaatcagcttctgaaaattggctgtttgcacggtcgtgtatttgcttggtagaaaatcaaattttccttcaaattagcattttctggtaactagagctgccccatcttcctctgagtggtctccaagtcagccaatagccttgtgctgtggcagccatgcctggctcttgatgctgtagccaaaagcaggcaggggatggtgaggctggtccagtccatggggagggacaaactcacagctctcagatcatctcagggcagcctttgttggcagaaataggtaggcagccaccctgaataggaggaaggcttctagactgggtcaggaggcctgggtttgcatcctagtggcaagcgtgcattcatttactagggctgccataacaaaataccactaactgggcagcttagacaacagccatttatatctcacagctctgaaggctggaagtccaaaatcaaggtgttggcagggccatgctccctctgaaacctgtaggtgcttgggcactccttgacttgtagatgcttcctgctgatccttcgtctgcacatggcattctgcctgtcttacatggccatcttataaggataccaactggattggattaggtgcctaccttgctcccatgtgacctcatctcaactaatcacatctgcaatgaccctgttcctaaacaaggccacattatgaggtacctggggttagcactctggtatcttttttcttgacagcacttctgacaccaaatgtgtgttttggttttttgttgttgttgttttggcaccaaccaattctcctatattaatgggttgtccaagaattcaattgaattctgacactatccagaattcacacagactccacgggttcagtcccacaaggcttccccgtcttcagatgccagctggaaatgtggtgcccaggctacccacacttttgccaaaatcctgtacttacaatcacagctttaaaatgaaggatgcagctcaggaactgccacatggaagagaagcacagtatggggtcgggggaagagtttctatgctctctctagacgcaccactctcccagcacctcaaagtgttcagcaacccaaaagctctccaaatcttgttgttcgagagtttttataaccctatctccagctccatactcccccattggaggttgagggttgggactgaaagttccattcttcacatgtgtggtgtttctggtgaccagtccccagaaactgcagctatcttggggctctaccctgagtcacatcattagcataaactcagatgtggtagaggaaggggcttattatgaataaaaaaagacactcctttctgccaggaaattccaagggttttaggagatctgtgccctgcacaggagctggggacaaagaccaagtatattttgtgttatgccacagaccccaacatgtctttttggagggagaccaaattcaacccatgacagtgactttgaacaagacatttgaacttagtctgttttttctatcctactagattgttggaaacagatataatagatgaaaattagttgattaaaattgaaatttgtgcataattcaaaagttttattttagccaagctaaagctttcatttattcaacagctatttactgagcagcacctgtgcatgaggctcagcagggccaggttctgggaacagagcggtggagataaagatccagacctgccccgaggaatagacagtccagtggcagcaaaggccatgaaacatacggcaactcttaaaaaaagccgagaccatgattttacaaaatcaacattttgtagggagcagaactttcaaagagaactggactagaaatttgggagtctttttcttggaaccctggtagatccagtagaatgagggatgggggtgtagggttaaaaacactgacattagaactggattacctgtgttggaattcctacatttctgtttcactatctgtgacggggggcagatggctgaatctcagtgtgcctctgtttcctttctcacaagaataatattactacctatctcctggggttgttttgaggtttagattatttaacacatggaaagcactcacagcaatgcctgccacagaaagaatatccagtacatcttagtgatgatcaccattattattatctgactcctggaaaaggacttgatttaattctctcatgaaacgttttcttggaaaactgatgtcaaccaagattattggtcttgctgttgcttataacaccccaaaaacatgactgtgtggataaaaatatgttggaaggggtagtctttctgggagcctgagaatagccatgtaataataactgcaaatatctatagttacaatttgaggttcaggtaaataaactctagatcttatagaactgcggtaaggtaggatagggagactccttcgactttctctgtttatttgtctctatttttaggagactatggaaccccacttccttggtactttcttctacaagagtcgtattggcttggcggtgaaggtgagtcctttaaaacacaaatcttaatgtttgaaatcaactccttgggctctgtgcaagatgtatatggatcacagaggtggccctctatgtaaacggtgtgattcctgatgagtcagctgcctcctggggctctgccccttgatgggcattgcagcgtctgggggaccacctttcacaagttgctgggccctgtgtgatcatgaatggctgatcatggatgaagccctgggtcctgtacaccttgtccagtagactaaattgccctatttaaaaaaggccaagccacttcagggttcaaagaacttttgcagcttttcagtataaagcagaaatccagggaatcatgaaggaacctttgcattcatctcccattgccttccttgtgcctttttattcttctctgccttttcaaaatataaattagtttattctcccaagatgaagactcctcctggggctgaggcagagctgttatcttcagggcaatacctcagattctcctggtgttgatctttcttaggggtggggaaaaaggctgaaagggcatttgcccacaacacatcttaggtaaaaggcacctttactactgaaccaaacaggaggcctagctagagaaagttctagaagcagggaaaagcacagactcttttgtgaggtctgagaaagcaaagaaattccagggtgaaagcgggggactcccctagagctgaagtactctcccatctgtttgttgctcacctacctattctttactttgtattattgggcctgggccaggacttatcctgcaagcactgagatggatgtttgttttctctgggggattagtctttttttttctttttttcttttgttttttgcttttgttttcactgggtcaaacaaacaacactttaacagctcaggattttttcattgtattgacttgtctacctgtaaacttgttaattttttactataataaaattatcatataataaatgaaaaatttcaacacagggcttgtgggcattttatttttctctacaatcccaacagatactctgcctcttaagaaaaaaagaaatcataaggaaaatatgctccttcaaaagtgaatcacaaatatgtttgccaacggaaggcaaatatttttcacctgtctcataggctggactgaaatggatttctaaaactctctaaaaccagaaaagagctgagtgtctccacccaacctccctcctttcacagattaaaaaataaaaaatggagcccaggagacatccagtatcttcccctattggtcacctgggacaaaatctggaacatgcacatgcattgcctggcaggaactcattccagtgattaaactcttcaggaggatgtttcctcttgctatttcattacctatttgtgcagtttgatagctagtaaagtgatcaaaggaactgtggggcatagattcaaaagtccttcaggaagcagaaatagaagaacagtactagaggcagcaggtccctgaccagcaggcccactacctgctgctccagcacacatcctgcacattttcagagggtgggggacagaggggccctgggtggctgttgcattgagaaatctcgccctgctcctgtatgtgcacttgaggccgagagcccttggatgcctggtgacagtggtttcctcctgcccctgccttcctctctggcagactgactggcccttctgctcctcttccccttccaggatgtcctgatatctttttaaaccaaatgccaagtttgccaaaaagtgtctgtttgtgtgtgtgtgtgtgtgtgtgttcaatgcgtgtgtttataccacacttcacaatttgtccaggcttgtattaataccatcaccaggctcaaccctggtgttaattccaagatacttaaatgcccatctaggtgaatttctcaggtaaaccatatattcaagctgtagtttaagctggctgcccgtcatagcactttgaatagactttgtttttgtttttgttttttgagacagagtctcactctgtcggccaggctggagtgcagtggcactatctcggctcactgcaacctccgcctcccgggttcaagcgattctcctgcctcagcctcctaagtagctgggattacaggtgagcgccaccccacccggctaatttttgtatttttagtagatacggggtttcaccatgttggtcagactggtctcgaactcctgacctcatgatacgcctacattggcctcccaaagtgctgggattacaggcgtgagccaccacatccggcccctgaatagacttttactcaaggttcaccatgactttcacatgttttgtattggagtaaaatgtgccagtggtgggctaaagaaaattaactcatttcaaattcaaacctggttttcttaatttttttaaaatcacagtttctgaaactgtgggctcctcatggcacattgagaggaggaggtgaaactctccaagtctgaagctcctgttataaatcttcctctggcaaagattgtgtgatcaggcttgagtacctcacagtcctagagcaggtcaaaggctggctaggaaactcatttgctccctgtacctctcccctcctttcctgcctttgctcgttctcagctcccggtggtagagtaacactggcttctgattggtgcagggtgttcaaccagagaagaaagagccctggaaaagaccgagcccctaacagaggaaacggaggatccagagcacccagaaggaatacacggtaaaaccccgataaagaatacacagcagaggcgaggaaaaggctctaagcactgcagagggccagagcaaaacatctcatggcaagggtggaaagaagcctaggaaactgactctctctgtggacaagtgttaaaccagatcccttctcagaggtccatctgcatgtgtgtggaatgaatggttcagcccagacattagcgcatatttcctggagaaagcaaataccaactatgtagtgtgcctgtgcccttgttaggcaaatcccaagtgagttgcacaaatgtgctgacttccgaggatttagcaagaacaataactttggtcactgggacttaaagcggatatgagctataaggaaagacaaaaataaatgcttctgtgtccagggggaaagagactccaggggagctgactacacttcacttacggcttacaaatctagaaggccattcattgaaaccatcagaagcctttcctgacagtggaagttacctaataatccctaaactgacgacccagatttacaagttttgttttcctggcttttgctgccctcatcttctctcttaaactagttctgtatttctcccaaggcttttcattccctaagcatacgcatttctctgtggccaaaatgctctgggtttagacaggcagcacagcccctgggctctgcctgacagggcaggagagggtctggcctttatccctccagcccaccccaggggccatttcataaaactaaagccagagacctgcagcccctcccagagttagactgcagtacaccatgcctctggcaagatcctcctcccacagtggaaagtctaagccaaatcaggaggctggggactggttccacctcagttgcaggcaaggccaggaggcacggatagaagaaacagtggactttttccccctagggaaagaaatgcttagagctacagtattaagatgacaaattaagctgtgccatatagggtgaaatgaagcagggatagatgggaggtcagggagaagtgagagcactcggtgagggtctgcactggagggggcatgggaggaagaaggaggggagtggggtttgagggatggtgatgaggaagcgtggactgccctacccacctattggaaaacctgggagttctgaggagcaagaagccttagtcaaagtcaactcaaagattcaagccaaggtgactaagagaatggcggtccagaaaaggtcatgggagaatctgaaggcagatgttgttttgggaagatgaagaacctaagccgcttccagaaattcatgaggaaatgccccgtggactgttggcaatgagggcctaggaccaaggttgagcttggggccaactctccctatagacagtgagtgcattctgacaagcatgggctctgggttcaaatcccaactctgccactcatgcctatgtgtccttaataggacgcttgatgtctctgtgtctaaggtttcctggactatggaaatgagcctaataaatgtctaccccttaggaccattgtaagagtacattgaggtaatttgtgtaaagcagtcgaagcagtgcctggcatataggaggtgctgtataaacgtttgatgctagtattactattattattctggagtcttccttgcaacggtgatagccgaagccacaggggcaggtgacgttataggcagaatacaagggcctggagacagagccctggggccatgtaattaggcattatgtttacatcatgttcattttttttcctccaagactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtgagtcttccagcagagaagctggctgccatgctagcctgtcatttcctggcttagtctttccctatcagcggctgtctactctttcccacaaattttagtgacaaatatttgcggccccaaaaatgtgtaaaagctttctgcagtattcaaagatcactaatatgtattctcttgatggggaggtagaatacgtttattgccccttttgtgtgccggggaagtggacattcattcagagagttgaagtgactttcctgaagccaccaagttgtcatggctcagcgggggcaaaagccaggcaccacagttgcctcttgtttctcacaccttgagtctttccccccatctcaacagtccatggtggtgatcaagtcatggccactgtcatcatgtgcatggaagctatagagtcctcctatttcctttctcttttcttttctttttttttttttttttttttgagatagtaaccattacccatgctggagggcagtggtgcgatcttggctcactgcaacctccgcctcccaggatcaagcgattctcccacctcagcctcccaagtaggtgggactacaggtgcataccaccatgcccagctaatttttgtattttttttttttttttttttttttagtacagacagggtttcaccatgttggccaggctggtctcgaactcctgacctcaggtgatctgcccgcctcagcttcccaaagtgctgggattacaggcgtgagcgaccgcaccaggccgagtcctgctattttcaaggaacattccttttcctaccaatcattaggcaggcttcaacatcagctgatgagggttagtggtcgttctggagaaagtgaaaaaagaatcagtctctagaggggcttgtggagtaaccgcctggtaacagaaggtcagggcagggaaggcaaaggggctctgcgcggatctctcagctccgcaggcgccccactctcctccaagggacccgagcgccatctgctgagaggagaacacggcccgccatggtttcccaaggagcagcagacacggacctcgcagggggcagcgaacccacgtgacacagtcttcaagtcctttggagagccccaggaaggaacaacagcgtgtacaccctgtgatggaatgttctctagggcggttcagtgtgaatggaatgtggggccggtgccattctaattggttctgtttccctctagtggttgatcgcggagatttcggcttctccatcaggacaagttcagatagcctgagatggtatcagaactcagggacagagctgggtgtggcggccctgcatccatctgctttctctccatgctaactgatatggtcagagagctggaagcaaattccaggaccccagggctccgcaaaggcaaacacattacttcatcggctgctgacatgcaacttcccccaggggttaaaacaatgtttaatactaacagtaataatatttttgagttttactttatgctggcgctgttctaatgttgtaagtgtattaactcatttaagccttacaacaacctaaggacatgggagtcatagttcccatttaaaaaaaaaaaaaaaaaaagcccaccattgctctgaggctttttatgttttggatccaaagctaatattggtggtggtaattcccatgcctggcttcgatcaattaatcagcaaatgcctaggactgcttagggttctggccttcatcaagaccttacccgggctttatgatgatgacacctggcttttcaatagccatgactgctcacccaggaggcaacgcctcgagtcatgcaccgaacaccttttattgatcctctccaacaccaggctccgtgatggctgagctggggacacctgtgactgcacgtgaacattttgaggctgggaatcccaaaggccctcggcgttggcctgggagcaccatgaaacaagtagaagcagagaaggatggcagaggtggccctctgcattagggcctggatgtatacactggtgctaagggggccccacagctaataggggtttgagtttgactgacagccccaggcaggaatctgtgagagttctcactgaacctggtgtgggggtggccctcctaaggcatgttgctaaaggccatctcttctgccactgacgcctgtgttctgcaggtccatcctgacgggtctgttgccaccaacctctgggactgtgctcgttgggggaagggacattgaaaccagcctggatgcagtccggcagagccttggcatgtgtccacagcacaacatcctgttccaccagtaagcgacacaggaactgagaccgccccatcccctctcctcacctctgcccccagcacacttctctagagcccagctcaggggtgccaggcctgggcacaggcagagatacagactcttatttggtttcccctatgtttaaagtcctttgtcctacttgcagtgagaattgtccctgagaatatgggactctgcctctgctgctcagagctgagggctcctccctcagaagggtgaggctgccttcgctctgacagagcagctgatcgatccccgagccccttgtgcagccctgaagtacttcctctctgggaccaaagacaggagaaccattgttcctttttcctgttgaagccacggcctgaaaggcaaacttttcagggggcttttcagttactttttttccccaataagatatcttttatttcttatctaagaagctacgcatagtcattgtgaaagaaaaaaaaggaagggaggaaggaagggaggaaggaaggaaggaaggaaggaaggaaagaagggagggagggaggggagaaggaagcgagggagggagggaggggagaaggaagggaacaggagggaggaaaagggaaggggaaggaggaaggaaagggaaggagggaggaagtaaatataggtaaacaaaaaattgaaaataaaagtcacctgtaatttcactactcagagataaccgctgagttataacattggtatataattttttagaactttctcctatacatgtatagatagataaacacatatacttcaaaatgataaagaatagtaaaactatgcatacaattttataacctgacttttttttcaaaaaaaaggattgcttttttaaacataagatatcaggaacatctttcatgtcattacatattcttctataaaataatatttaatgtttacagattattccattgtatgcatgaactatgtaagccatcctcttattagatatttaagcagggtctgctatttttgtattgtatcataaacaccaccacagtgagcatcttgattgccaaatcaagaatacttgtcctcaattatttctgtaagatcagctgctggaagtggaagtgctaagccactgcttttctcgttgtcccatcctcctagcctcacggtggctgagcacatgctgttctatgcccagctgaaaggaaagtcccaggaggaggcccagctggagatggaagccatgttggaggacacaggcctccaccacaagcggaatgaagaggctcaggacctatcaggtgctcagagctggatggagacagggccacagatggcaaatccatggctccccagtgcacccaggaggcaggggaggcttggagcaggagagcttctaagggtgggaacacctctgtgaagttacaccaaaaatctaagagcagcccccagatcattttccctgcagagcactgtctcacagcagcctgggttttatttgtcctgagattgatgtgcttgaacagtcttcaaagggtctgatccgaggaggtgagggttgccctttctgcatttacaaagcctgaacagtattagggctttgaacgctataaacatctaagaggcagcaccaaaccactgctgggttaaggtacccccacaatgccacttgccctgggcctttctcttcctcaccctccacagccccttaactctcccgtccttcttgtgcctccaggtggcatgcagagaaagctgtcggttgccattgcctttgtgggagatgccaaggtggtgattctggacgaacccacctctggggtggacccttactcgagacgctcaatctgggatctgctcctgaagtatcgctcaggtaacagctgctgctcagtctcctgggctgggctctcactgcagccctagctgtggtccccactctctcacctgccattttgtagctgagtacaggaaccacaatgactacactcagaagggggtttatcagtgacttggtgaatctaagttccagctaaagcctcctgaggtttttacaaatataaacagagaatcactgatgatgcaacctacttcccaaaatattttagaaaattctcttgacctgcagcccttctgtctggaataatggatgctactctaggtgaatgtcttctctgaccatggggacccaggtcacctgcaaacatacctagaagctccatagctgtcagatgaccactcaggaccagtgtgagggtgacctgctgggcattcagtgctccagagggtggccacagatggaagtggctcctctgtcatggcacctctcagacaaggggctcagatcagaagagacagcaagcagagctgagtgcccatagaggtaacagcacggttcaaccccgtggtcaagccagagctttcccccttgctctactcacacagcgttgccccgtgcctttctctgagggtttgtcatcctgaaatcctcattgctattttctttctttcttttcttttttttttttttttttttttgagacagaatctcgctctgtcgcgcaggctggagtgcagtggcgcaatctccactcactgcaagctccgcctcctgggttcgagccattctcctgcctcagcctcctgagtagctgggactacaggtgcccgccaccacgcctagctaattgtttttgtatttttagtagagacggggtttcaccgtgttagccaggatggtctcgatctcccgacctcaggtgatcctcccgccttgtcctcccaaagtgctgggattacaggcatgagccaccgtgcccggcctgctgttttctgttaatgacatctccagttagtgagagtatgcacgtgtgtgttctttatgaagagtataaatccagagcttaatgatccagaaaatgtacatatgaaactccctagatgctgaccataatacatgagcccctaatatagagatttatttgaatcagatcctatgctggatacagagacactgtgtgtggcaatgctttacagtatgtaggaagctatgaaatgttagttattattgtcctaatatgctggaatttgctgctgaattagttcccttgggtttttttttttagttaactcctgatttttgcaactatatagccaggaaattgctgtacaccctttaccaacaatgcccaacccagggcaggcctggtgattgccctggcccctaccttgcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggtaggtgtctgcccagggaaggaccctggcctgggtgagaaggagcacacagcacggggctgccactccagacatggctactcacacaggctctcgccaccagaatcagtgtctttgttctgggaccatttgcagaagatttcgatgaacacattctgaagcctcctcctacagagatgctttagccaaaatgaaacaactagctttaaatggtctgcaagtattacatgccagattacacaccagtttggtgcggtttggtgcaacatagaagtgagtgtcttattctgtaaggttaggctgttttaagagcaattggttgagcttcatttcaacattaatattccctaattaaacctgaatttcagtggtaagtgaaaactaagaagaggcctccttgggtgctataacataaaaatgatgaaggcaaaaagtaccaaccagcagagaccacttcagcacatcaggagacccagttttatgtctgtgctgcgaagtgaacaaactgtgtcatcctaggcaaattatttaattcctccttttttttagtatttttttcttcttcacatggaacatgaagctaatgacctctgcttctatttcttagggatgtgaagataagtgagataaagtattataaatgtgctctgggcttcttaagaacaggcattgctcacattcaaatggtcatgattatgatatggcagcattatttatgcctctggtttaagtgtctggctgccgctggggtttcctatgtccatccacggggagggaggcacagaatgtctcccacaggcagaacctacagctgccacataattgatgacaagccaaagggacccttggaggttctgctcctctctgtgtgtgactcacacactctctaggataaaatcaagcgactacaccctcaaaatgctcagatgaattaacagattaaacagtgaagaaaaaaatgtgttgactacacttggcagtgagaaataaataaagcgggcggtgacagcagctggcatcagggagaggctgtcatggaagggatgtgcatcttgtcagtcatcccatccatctgttgcaggggacctgcagctgctcgtctaagggtttctccaccacgtgtccagcccacgtcgatgacctaactccagaacaagtcctggatggtaaggactggacgggccatacttgggttccgtctggcagccatctcccagtattgctgggtgtgtcctgttgtgatgcattttaatgggagcaaagagaacactgggtacttctgcaggtcacacagttgttcttttgctttgagcttctttctcctcttccttcttccttcattcccaaagggattttaaaagtcatgcacctaaaggccctctccctttaatgaggaatacactctgtgctcttacccttagtaagccatcattcctggggtccccctgccctggctccaggccacattccttagtgtctggggagagcttcttctacatgtgtgccgtggcgccctctagtggaagcatggtgatgcacggctcttccagtgaattcgtggagtcagagattgcacatgtggatggcaagtctggaaatagcatacacccctgttatactcctgattctcccctcagcttcccaatttcccagtgattctccctttaattaggatgcactgaagctctcaggggtgcccccatctccaaggagctgcagtggagaggctatcccctctctatgtgagagaatgtgtgagaagcgtattcccacacaggagcaaaactaaacttacgtactgatgcaggttaatgaatggggaaagtatctgcttatcaaagaaaaggcatatttttctatttagcacaaactttttcaaatgttaagaatttactaactgaaatctggtgaagcaagagaaccgggcaatatttgcgttgtctgatcattacaactggagggaacatgctcagagaggcatcatcactgttcatgcacctgccctctctttacactgagagaccctgtgatgaacagaaaacatctttttaggatgacatctctgggtctttctcctagcctgccttgctgtgggtacctatctccctgctctctgaaccttggtcaagaagtttatatttgttttaaattgatactaatatgttaagttactgtgatttgccaaaatcagattggaaacagggcctgcatggctgaatgattcttttttttaaattactttatttctaaataaaggttttctttgtatagaatcgggatgctgtgaatggtgggaaatgcactaaatagttatgccccaaataagaaagggaaaatcatttgaatccccagttagctccttgaaagtcttttcacttaaacacacccacataccacacacacactcacagacctccctcccagatgcccaaagccctgctgacctacagagctacttctggaaaggctgacacatgcctaagacacaattcctgggaatccagcagctttgggttcaatttccttcctaaaagaacaatgaatatgacccctggagagctattagggcagagctgcttccttaacgtaaaggactctccagcctccgtatgaagtcatctcagagctaaagacaatcaagtccaacttgcagatttgacataaagcaagacttccaatccggctaggcagaaggattttggttgaaaaccatgaaatcccttcatatggatcattttttaaacaacaaaaaaagaaaagaacctactgggtgtccacaactctgagagctgctttctgaagagtcatgttttgagtcctggaatccctctccctttgacctgcctctcaagacaatgtgcgagagaactctctcttcaagtgcatgcaagtgaggttttcacagttagatttttaattttaaagtaatacacatttgtacataaaattcaattctgactgtatacatgtgtcagataaacagttgatacctgacacttgttcacagtctatgatacgcaccgcatatcctaccctctcccccagcctctctccatggcttctcaaccccccctctgcatttcctgtgacctgaggattcagttttgtttgtggaggcaggtgcaatcccaagagaaactgtgcaatcttctgagaagttagagtaggcatgtgtgtgtgatttagggaaggtacttctcactcagcttggtcaccggttccaggtttgtgtcttgggcaagtcccccatagctggtgacagaccagaaaaatgaaaacaactttgacttagccctcaagttttcagtgaatgagaatgaaaaacaaccatgagtaagagatttcttaccgagatgatgtaaaggataataatagcagccagcactcacctatgtgccaggtatttctctaactgctttgtgtagtttgactcatccagtcctcaaaaacaacaatgaagtggataccagtattttccccttttcacagatgaggaaagtctaatgtgacccacccaacataacatagtttgaggggacagagcatttcgttgaacagaggaggaactggcacaggaaagttgcatgacccccccaccaacctccgcccccaggttgcacagctagctagtcgggaggactttgcttccgtttccctctgcctctcaatgatgatctcagggccaactaagctaaaagcagacttgatggagcatcagtcctctgaaagagtcactgccgagatacaaaatacctcttcttcaaaggggaagtggagagaagtaggaaatctgggtaacctcacagtcttccagtttctggaaaacagagctggcatcagtcttttttcttgtcctaggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagaggtaaagtagagattccagctggtttctgtcaagtgccagaagtggcggttctttgaaaaagtctaacattagagcaaagttttgtaaaagcaaaaagccatcgttccccacccaagcatagcaactatctttatttttggcatagttcccccatctctgcatgcatacaaattttatgtacttgtggttactgtgtgcttacgtttttgtatttatagaagatgatgttctcagatagagtcgtaatggattttcttcccattatgaagcaatacccaacaaaacagagcttgggttagatttttctgagaataagaatgactaaacaaaattctctctttttttcttcttgacagatttttctgaaggtcacggaggattctgattcaggacctctgtttgcgggtatggtgctggagccagtggcttgttcccttccttgcctccctcccaagttccatctcgaaagtctaaggggctgggcacagtggctcatgcctgtaatcccagcaatttgggaggccaaggcagatggaccacctgagttcgagaccagcctggccaacatggtgaaaccccatctgtactaaaaatacaaaaattagctaggtgtggtggcgcgcacctgtaattccagctactcgggaggctgaggcaggagaatcacttgaacctgggaggcagaggttgcagtgagcagagattgtgccactgcactgcagcctgagcgacaagagcaaaatccatctcaaaaaaaaaaaaaagtctaaggaaaaagtcatgaaacaacaaagcaggcaaatactcctccatagtatctgactccccagtagtaggcattttgcatcctagatggctttgagtgacaaaggaataacagactgagttaggtctagatggggacactttggatgaatgaggattcttacggaggtcaggttggtagcttcatccctcagctcctcatgctgtatccccagtctctcggcctgccatgtcatcatcctcatctcctcctgtcatctccaccaggcctctgatccatctctgtctgcatgagtgacagctggcagagtccttaatgtttatcaaatacaactcagacgtcagtctcctggcccctttgagatcaacataaaatcattttgaacccttatttagtggtctatgggctttgaaaacatggggaccaaaattcctgtggattctagaagtctctcttctacatgtgtcagcctgggcaccaactagctccttccatgaacttttatcaaacccacagccacacaaagcatgtgtgagtgtagcagagtttacagcagagggtggagggtggggagatagatgtgtggaagggttacctgccacacaaacagaaaccacttctgatagaacacgaggtgtccacccacactgtaaaatcctctcctggtacaggcaaagctttgcagcgattctcctttgctgcccctgggctcctaacacctcctaaaccaccagttacctccttctttccagtgtggcatatttcagtgttttcctgttggagtgtttcctttctatgtggattctggaatcagctcttaagataacttggttttcatctttcttcataatgatcccaaacatctatctactatgcctagaactaccaatggacacatataccagcccagatatgcttcagcccatcccagtacatcgcatggtgaccaaaagatgtagtcgtcctggcacagtgggtgtggggcaggaagcagtcctctccaggggacagcagcaattcaccacagaacccaagtttctttcaagctctgctgacacagaaattgaataatctcagctcacccaatgtcaaagactcatattaaccaagaccagaatgaaaatatgctaatttatatcagaagctttgctggattcaagagttagggccttttacctgtgcagaatattccttcttgataaataggccctctcaggagaataaattacacatcagaggactgtttagtcagcataggcatagaacaggatgttccaaagatacagtcaaggggagtgggtaagagtgtagcctctggagtgaggccgaccaaatatcaaacctgagcttcataatttgcaaactaactggctttgggtaagtacatagcctctttgtacctgtttccccatctgcaaaatggagataataatagcatctacctgtagcattgttgagagaattaagtgagttaatgcttgccgacttataacacagtatacgatcactgattaagacttagcaactctaaactaaatgtttacaaaccatctcttacctcaaagcacttaacatccattgtcttatttgattatcactgtaatcttatgaagcaggcagggcaggggtctgccccatctggggggaactgagctcacagaggttggagggtttgcctaaagtcacccaggccactgggtctcactctctggtcttagctctgtaatctaggatgctcaatgccacactctcagccacttttcagatggctaagtacatttgttttgagttagctcagtctcagaggatgacattttctgatcttgtctccagtgtttaaatgaacctgtagctgtgcattggggtcacacaatgcgtggcatggagagggtctgtggctgactgccacggttactacgtgaaaccatcattacagcagttactactgttactgcctgagaacatcattacaagactgaacgaagggatcaacatggaaatgataacaaaaaaaccaaagtaactgttttaaggaaaggctagcatcgggaagaagaagagagaagaagagaagaagaaaagggctccctgcttctaatgagtaaaggcagctccctaagcttctgcagcccttcattatttattgggtaacaggaggaaggagcaggaggtaatgattgggtcagctgcttaaatgatcacgggttcatgttgttactgacagatttcaattatgcctaatcataagaaacatttgtgcagcctccaacaagggtcaatgccacttctgaaggggtgactcatagtcagtaactagaaagcagcagatagctagggacaaactggcgattctgaataggcctggaacccttagctctggccaggtcagtgggctccagtcaggatggagccttcagggagagatcaaagctcagaggtttgagatgatatcagccagcaaagaggaggggcagtagggatcctcccagagggagggccagccatagaagacatcaaatctgagcccggatcaggagaaggagcctgcagaactggggctctggcaccgagaacctgcagaacttcgcccctctgagtgcaggtgccagggctggggctgccacccagccttcgcatcccaggcctggcacgtcataggtaaatgtagttgaaaggatgactgagctgatccaattccctttacaactgtccttgtcctgggggacttgaggagggttaagaaagcagctggggaccaaccaacagtcctctaggctctccatgtccagcaatagttgttcagcaaatgagcattaatcagtgactataaactgtagcttcaacataaccgacaacttgcaatggtttctagagcatgctcccatgtgttatctcatttaaatttccaaaccaatcctgtgaaatgttctttttttttttcttttttttttttttgagatagagttttgctctgtcacccaggctggaatacagcggctcgatcatagctcactgcagccttgacctcctgggcccaaggggtcctcccacctcagcctcccaagtagctgggactacaggcacacgccaccgtgcctggctaatttcttttctagttgtttgtagagacagggtctccctatgttgtacaggctgatctgaaactcctggggtcaatcaatcctcctggcttggcctcccaaagtgctgggattacaggcatgagccaccatgccttcattttacagataagaagtctgagaaaactcagatttaggcagattgagtcacttccccaaatttatgtatcttgtaagaatccatattcaaacctcagtcccctaactcttagttcattactttttctaccacttctcagtatcctctaagaattcagaaagaaccacatcgactctgatttttcatttgtttaagtacacaggtaataggtgaatgtattttgttgtttaaaaattcatataatacacaaaaggctaaagtctcgcttcccacttcctctcccctttctacccaactctgcctccccagggagagcttctgctgacagtcggtggacattctttcagagttttacaattatgtgtgtgtgtgtacataagatgtcagtttttctttgtgtaggatacatgaacatgaattttaaacataaatgtgagtgtattacacatattgaccagcaccttagtttttttgtttgtttgtttggttttctttgtgctgtttgagaaggagtcttgctctgtcacccaggctggagtgcagtcttgcaatctcggcttacgcaacctccacctcctgggttcaagtgattctcctgcctcagcctcccgagtagttgggattacaggtgcctgccaccatgcctggctaatttttgtatttttgtagagagggggtttcactatgtaggtcaagctggtctcaaactgctgacctcaaatgatccatccacctcagcctcccaaagtgctgagatgacaggcgtgagcctccgtgcccagccagttttgtttttttattaaccaagttacgtattttaaacttctccatgtcaatgcttttagagctattttgttctctttaatgttaatagagaattttaaggcaatttcaggtgaatctatacaatttctctgtataagtaatttacactagaaatagatttttataaagatgattaagctaccagcctggtatttcattgctgacttaaatgaagaggaaaatcaatgctgtaagggaaaaaaaaaatggcattagagatccagaccttataggcattttccaaattattaattcaatctctcaaaacaggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcaggtactattgtcggtcggtgtttagctgagctcagtggctcctctcccagccttcccctcctctcctgagtgttccttcaggcatgggttataactcagcaaggagcaccctctttagattctgctggttttgtttcctgctttccaaacccttatcttgattcttggtaacatgaatcttctttgtaagttggacctcccctagcaaagaaaatagaataatagtgaaaatgttaatattgtttttatttttacagtgagggataaagtcatgttttcattcatttttgcagtgaccctacatatcaaaatcattgccctcttttttcttttaatgttgtttaatttagaaaaagaagctctggtttaaagaacagtgagtcacgtgacttgctctttgaaatgccctttgaagtctggctgaacactgggctgcattcagattcttcagtggccaccagaacattctgttttcttctgcacatcttacctttgcacaccctgcttattatgttcccccagaagcccaaccctctccaccaggggctgattaggaggctgcaggataaatgtttaaaagaatgaagatgtgtgtgcacgcgcacgtgtgacatctccatgccacagtcatgtttattccacgtctattctcccacagatcgtgctcccggctacctttgtgtttttggctctgatgctttctattgttatccctccttttggcgaataccccgctttgacccttcacccctggatatatgggcagcagtacaccttcttcaggtgcgcggactcggggtcaccattctcctctgtgggtttggggcacctgggtcacatgctgcttagaagggccctgaccttcccacttcactgggaccttcaccaatgagagaggggaggggtctttgggctgcctgcagaaaggaacttaatgtatctgccactgcttggaaaggcgatcctagtggacaggcaggactgcttgggaaggccgaatggggaaaggaatgcaaagcttaggtgaatgggttgaagcgccatctttttgaggcataggtgacatgccatcagaccactgcgagtgttcaggcagcctaccgcactcccaggagagctagcgccatcccaaggcagcattcggtgcctccaatacatacctggcacacagcagctatccagtaaaggctctgagttgcatgatgttggcacgcgcctgctctgtcccagtcacatgtctcactctgtctagcatggatgaaccaggcagtgagcagttcacggtacttgcagacgtcctcctgaataagccaggctttggcaaccgctgcctgaaggaagggtggcttccgtaagtgcctacgcgcccctgtcctaagaagactagctcccctgggaggacccaacggtgggttcaagatggcaggcgttggggaggccccactcaatcctgctctgctggtcacttccatgtctctgaccagcactcccccaacctctccttccacacttgtgtgcagggacattcactacctcctaggaagcccccacaccactggacagctctatatttctcagcatagaagttctatgttgagttgacagatgattccccataacttatttgaaaggcctctgagcagggagggagggaaatagggttatgctattgtgtgattgggccttgaatggcgtgagtgacacagtggccagtactttgtgatagttgtgagtctggagaagggagttagcgaaggccattgacatccaccaggaatcctaaaagttcaatataattttaacttttctccctcagtctttttcaaagctgtcaataaggaccaaaacagactaatttcaaattcctcttctggttgctgtgtctctcaacagctagagctgctaggaataaaaagggagacaaaacgatccacaagctagagatggttattccccagccccacacctagtcagtcacaaaaccctagttttgatattgcttgagcagaaaccagcctccaagagaataagaagaaagggcctgggtctaaagaggaggaggaaagggttgggcacaatttcttatgcctagggatttgtcagcaactttgaggctgattatggaatattttcttgtcttccatgagggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccaggtacctgacctccaaacaacggggccccaggtctgcctgccacagagggactaggggagtccctggtatctcctgagtctctcacaaactaacatttcaaactggcagttgagtaggggactaaaccaaactccctgcaccctctgggaggggctccccacagggcgctgtggctgccaactggaggaagccactcaccaaaagcttcattttccaccagatacttcctatttgatctagtagaaaaaatgtgtttaagcactaaaaaaaattaagtcatatgtgctcattatagaaaaattagaaaacacaggtaagtcagaaggaaaaaaaatcatcgcttggatataaacacagataatgtttggtttgcagccacccaaacagattatattccaaatattgtcttaaaatctgatttactgcataatttactaggaacatgcatccatgtcaataaatagacatctgcatcacttttaatatctgtatattatcccattgtttgaatttcttttttttttttttttttttttttgagacagagtctctctctgtcacccaggttggagtgcagcggtgtgatctcggctcactgcaacctctgcctcccaggttcaattcttgtgcctcagcccccccgagtagtggggattacaggcatgcaccatcatgcccgcctaatttttttggtagttttagtacagatggggttttaccatgttggccaggctggtgttgaactcctggcctcaagtgatctacccacttctgcctaccagagtgctaggattacaagcgtcagccactgctcctggcctaaagttactttaaattaactgatctcccattattcgccacttaggttttttagttttcaccattataagcaatgctatgatgtacattcaaatggaaatgtgtttacacacttattaacagtcttaattaagaagctctccatgtgctgtgtctctaacatctgcaggtatgtacacaaatacatgcacagccagcatccatcttttgcagggacattaatgatcttggctctgagcagcaccctgtcctgggagttctaaagtccagaacagattacagtgagcatctcctgggggatttagagacatcaaagaaggctgtgtccgtggttgataatgggcctcccagctgacttgccagggctgggccttagacagccctgtccaatgatttgtcaatgaataaactgttcccaaacaggctatgcagttcagtgggaaagcacaggtatgggacacggagagccccaggtggactacttgacctctctgagccttaattttatcacctgtgaattgggaataactgcttatttcataatattattatgaggatttaatgaaatcatgtgggcaaggaattatttagaattagattcaactcaagtgatgacaaccccaaactaacagcagataaaacaagacacaacttgtttctcactcatctaaaagtctacgtgggtggtgcacgatgttctattctctttctcctccacactaaacaggcctcagcctcatcagccaataaggcaggagctgccttccaggcagcggaatggaagaaggatgaagcaaaacagagggcagagtgtgcacatgtgctatgtttagggaaggttttctgaagttcccacatagtacttccacttacaaacccaacaaaaaaggctatggctaaggcagcagggaggagcaaataatgggagcaactagattttgccacagcacctatcacagtctggtttataaatggttctaggccaagaacacccgatccctgctcttttttatattctaaagcatgtatctttatatttctcaagcaatattttctctctttgaatcacagctcatctgctgcatcatagggatcccaaaagaaggacccaaggaacttgtctcagtcctctgtgccccaagaggaagctttgcttgtttgctttgctgtcaatgctgagggctcctgtggctgcctccactcaaaaccctccagcatcaggacgtcaaggctgtgatactgtaccctgagctcttggccagggcgagggaggggaggccaagcctacctacatggtgtttcatttcctaaacgaacccttacttccacgcggtctgtccagcttagaaacttattttcagtagtgttggtccttggtccctggacaaaatgtaacagccaaagtcctagaaaaaggcaagccagttcctgccattttctttcacttctgcatttcctcactattatacgtgccttccattggagcaaaactgaatgccacgcatatgcacaggagctgtgcgcgctctgtctctctcactcactctttttctctctctctctttctctctcaatctctctgtctctatctatctcttactctttatctctcactctctcactctttctcactctttctctcaatctctttctcattctctctctatctttctctctctctctctttctcacacacacacactcacaaacccacactcttattcacatctgctcaccctagccactcaaacacaatccctcattcagcctggaataagtccagagggcgtgggcctgattcagagacaatcagttgttctcatctgggaaatggggcaatgtggtcatctctagggaccctccctgctctaacattctttgaatgtggtgggtcctgaggtggaagcactctgtccctgacttctagtatatgtggagatagggttacacaaatattttattgggcagaacttttataaaacaatttatcataagctatcgcagccagcagcaatttttccaacctggattccaccaggggagcttggccggtgtctgagtgccactttcagcttgagaagcaggtgactcagtgaaaagagcaaggaggagacagaggcagattcagttcctaggccctgggccacccacctgcaagtttgcagcccagtcagtgcaagtcagctaactgttctgaacctcagtttctctgtctgtaaattaagctaaaaattcttctttcaaagagtgtcaggatgaagtgagatcgtgtatgtagggcatttaacatagtgcccgacacacagggagcattcggtaggtgccagctctcctcctggcaggagagagagaaacaaggtgaaaagagtgaattaaagaagaggaaagtcaaatgggaaaacagggggaggagatagaaagtgtatgaaaaggaaagaatggtgcgcaataacggcggtgtaatgccaccaaaatcccctcaactacttctgggcagcacccttgacagagtgaatgcttttatgagaatgtaagcggaatgtgttcccagatttgcagtaatattgccacctggtggacaaacccatgcacctttgaattttccaaaatatttcgatgaactagcttccagtcctagatgtattttgaaagtgatttgtaaattgtaaggaactattcaaattctttcattaatgtcacaaatcaactgtgtcatctgtatgccacccactattctgggtgctggggacacaacagctcacaaatcaggcaaagtccctgctctcaccaaaatgatatcctacgggggattacagatacaaatacgtaaacagatccatcgggaggaaactctcagatggaaatgagagctatgaagataacacaacagtacatgacaatacagagtgactggaaccaggaacatttctccgaggaataaaatttgaagcgagccatgagagggtctacaggtagagttcccaggcagagtgaacagccaagcacaaagctgcaccaggagagagaggtgctcgccgagagacagggaggggagtgtggcaggtgagctcagagaggggcagggccacacacatcggccacatgggccttggtagtgagtcgagatttgatcccagggtttattggagtggataagtaagcaaggtgactgaggtgctcgggtttacatttttatagttcaagctggctgctgggtggaaaacggaagttggcagaccaaggacagaatcaggcagacccatgtggaagtttctctagtggtctaggtggtggcttgggtagcgtggcagtattggagctggagaaacgcagatggattggagatttgttttggagtgacgccattctgtcttgtcaatggattggcgaaaaaagaggcatcaaagatgagttacacatcattgaagtgagaactagggagatgccagtactttatttagtattttctcagcagctcaatccataaataatttttggaagacaacaagcagtttcacaaactacttataagtcctcaagttccaaggtaattaacgtgggtgtctcattgcctcagagaacacagcgcagcacggaaattctacaagacctgacggacaggaacatctccgacttcttggtaaaaacgtatcctgctcttataagaagcaggtaagaagaaatccttttatgctttttatcctggctccctgtagaagatattaactagggacagaagataattttctctctcaatttatgtatgatcagggcagtagatttttttcttttttatctgatttgagggccccattcaacataaaaagcaattgaggcacatacaagtaaaatgtaacttaagattaattctttttttgttgtttgtttgtttgtttttacatttagggcaagcagtcttaaattttaacccacgtattattaaaagttatatcagaagaccatagaagttattcaaaaatgcagccacatattttaactagttaaaagagagagtaaaaatttggagggaggtggaggagtataggggaaaaggtagaagaaaaagagaaaataagtaagtggcaaaaaagagaaaggaaaaagatagggtgggaaagaggcagcgggacagtgtctgagtccagcacacgccagggcgagccaggtcaactgcagctgtcatattctaactgtgaattatcatctttgatcactgccctttgagatgccaatgaacttttcaagaaatatctagttctcttggctctccagctgttcttatcagccccatccaggatggaacagctttggcagcccgtatcagaacaagcagcttgacaggggcatgccatgccaggagagaggatcctaaggaagcgtggtccagtccgcacaggctctggggctttaagataaaacctcctgtctaactttagtaggactttctgttgcttcacctgccagagccctgaacgagggataaattgacttaattaactagaacacactgcaaatggtgaaagcatttagcaaaacaaagaatgccatccaagccccaaaataaaagcagaataaatagaatgcaataaacagcaaccatcccaaactgagttctcagcagcaaatctccagtatgaaattttggattttgtgcgtgtgtgcttaaaggtggatgacaatgacagttcatgggattgagctctggggtccagagttggcatctgttcatttcccattttgtcattttacccttgattgactgaatgtcagtgccttaactttgggctgtggagtgagtcggaactcccccgaggtgtgcaggtggttgttagagtctcatttttgcagggtggaagacaggagggctgcagccttcattccacactgacatggtcattgccgtgtgttctgggtccagatcaggcatattgacctgacatatgacctgacaacaggaccactcagaaagtccagcatgcgggatatgatttggagagccagtgggggaaatcataggtcctttctctgcatgtgtattcaggcaatgtcccagggctgggcggcttccgcattgcttggatatcggaaaatgcaaaaatgcccctgaagactgagacttcagtcttcaaaatgaatgtttgggaaagaaagttaacggcactgctgtacttgtggtattcattgcattattttattttggctttcagcttaaagagcaaattctgggtcaatgaacagaggtaagaaactatttttatcagaattaaaatctcagattgattcattgttgaaataattgcacacttttaaaaggcacacctcacagccatgaggaggggctgttctgtaggtgctcaggaagtcacaagacacgtcctgaagaatatgtggctagggacatcccagactcagaagacactcagtggtgcctcttcttggaggacataagtgggggtggcattccctgatgtggcgtttcagagcattctcacccaaaaaaagcttctaaaacctccaagtatataacagtttataatactccaacaagagggccttgtagcctaaacccgggacactccttggcccattccttttaagcttcagggagtgtgggccagccccagactcaccccattcctgaggcatcctggaggttgaaatatttccagaggtttagaacctcaccaagtgggactctaggagcctgctgcctcccagcctccctcaggaactgcacctccagaacaggtgcggggctgacatgtatgtgctttcctgggcagattctagaccgtacacatgaaatctggctttcaggattgctctccagagggacctgtggggcctcggctgagacagagagtaggagtgaggcagtgattcaaggccctgagaaagagctcctcctctgcttggtataaccagctaattcattctgttctgttgactttggcttctgccctgcctttgaagggtttgaggccagggagtgatgcactcagactggtgtttccacacagtcacttcagacttccagggcagtacaggagatagatcccagggccagtgaagaagcagagcacaagtccaggcaggagaggctaagggcctccctgaacaggtgtgaggcacagaagccccgagaggtagggatgacaggatgaagatgggtcctgtgctgctagaagtacctgcaaagcacagaggtggcacagaaaaggagtccttggctgggatgggaggagatgacatgtgacatgtgaaagaggacctggagttggctcgatgctcccaaaagggaaaggtgccgaggggagctagcagccatgcaaaggcagagacatgcaggcagtctgggccatgaggagctctggaagtgactcgatatgtccagaataggccactccagggaagggctgaggaaggatgaagttggagaggggcacagaccagatgcagaagggcctcagaggccaggatgagggtttggactccttcctggaggcagcagcagtgggaaaagagttaaaagctggtttgtaaagtggagccatgttgctcgctggtccaggcaattcccccgaaagttcatgtttccctacaaaacccgagagagctactagtaggcgtgaagttcgtggccctggtctgaggatttcctgtttccttgtcaggtatggaggaatttccattggaggaaagctcccagtcgtccccatcacgggggaagcacttgttgggtttttaagcgaccttggccggatcatgaatgtgagcggggtatgtaaacagactggagatttgagtaggatttttgacttgcttaactaccatgaatgagaaactctcatgagtgataacaggaaaaaaaaattaaaaccgtcttgtttgtttgtttacatggtttttagggccctatcactagagaggcctctaaagaaatacctgatttccttaaacatctagaaactgaagacaacattaaggtacttgacctatgtataatctgctctggagctaaaaatttacctgagctggttattttatttttactttcctaccttcattaaattccatccctcctcctgctgaaatctagcaaggaatgtcttccagctaccaaacccttcctgcttctcaaatttcctttccttcactgatttctgctttaactagctgttagtgcagcgtctcagatgtcctctccaccctctaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgtaagccaccacagccccagcctcaccactttcttgtcaccttctccactctttgaacatcctgagaggattctcaccaccgcgaagtgctgatttggatggtaatgctgtttagtcaggcacatatgaacatccgactttcaaataagtgcctcacacttcacataccagacctcttggtcattctttctccccaacatttatgtggcaagtaagtttacatttggttccattcccttttggcttttgatagcaagttgctcctggagcttatacaattattatctttgctatgtgcaaagcagctgccaggaactggcaaagttcagtaaacctttcagctccctcggagtaattatcttagattccaggaatttcctcagaagagcatactttggagatgtcgacagagctttgctaccctcaagctgaggctcttcttgcacagtttcagccagtggagacagtggccttgtgcgttttgtagtatgttcactctatttgaggcctacatggaggaggggttggtaggagcacctttgttagtgcaaacttcagcaacgttgtggggtcctgattttactatcctagcacacgctgagtgccagtgaacatgcccagggtcatccactaaaacctgggccttggctccttggtgtcttcctctggacaccctagggccctagactgtcctctgttaattctcactcagccacactttcgtgtgtctccttccagtcatttgttctaagcttactacgtgtatggatgatatgatctgtagttttatcaaggtagtgactaccacataggatacctttgtggaaattagtaaaaatgctcttttctgcaggtggacactgtcccatgccaggggttatggcttgtacataaagttcaggctggctttagccccaacttacccctcagccagatgccttctatttgtccgaggaaagaataaatagagccaagtccctgtacaacttgcctgccctcttttcacttaaatttacatcatgaacatttccttgtgttacgatgtacttcttgaaaatgtgatttaacaagatgattattaacaaaagataaatctcacagaccgtatgtctgtcaacatagaaaattcaagagactctatagacagattattagagctaatgagagcattgcagtacataagattaatataaacatctatttctatacaccataaaaataattagagaatataataaaaagaaaggttgtctagaaatattcacatgaaatagaaaggcaacccgcaaatacccatttaaccttggtccatatggattaagacagtttagtggagtgacagcttcaaggtagagaagaggaacctggaggccacacctgggcgggtgtaaggccttcccaaagcctgactttgtatcttctcctccttctgctcttccctcttcatcgccctctccctgtgtctctggccctgctgcaggctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcgtaagtgtcagtttacagcgcctccctcccctccgtgggcccaaggtggagcttgtgtgtgctctgaaggaccagaccaagaggggaggggttctcacggtgccagggctgctgaaaggcactgggccaagggccttgtgtatctgctgtcccttgacatcttctcagaaaggcacagaactaggagcccgaagctaggaaaggctgtggggtgcagcttaacaactggtgaacgggggctctctatgtcctgcactgaggggtcttctgacccatcaaataatcactgcaccgcaggcatgagtctggccttcctggcatcagtctggcgctgagaaggtaatatgaaggggtctttcaccccaagtccccttctcaaatcctgccccaccttcaaaagggtaaaggtaaaactttccctgtggtagggtcaccagataaatacaggacacccagttaaatttaatttcagatgatgaataatttttagtataagcatatgctacttcaaatattgcacaggacatatctacactaaaaaaaaaaaaaaaaaaaaaaaaaacctggttgtttatctgaaactcaaatttcactaggcatcctagatttttatttgccaaatctggcaaccccagccagtggccaaaataataagaccttcacttattagattaaccaccgctacagggaaaaatgaagaaaaaatatttattaaatcaatagcacactaccaccttcctgacaaccaaggttggtgggggtagggaggggtcaggatagcgtaccctattacaggctgcagggtcaaaggaattggtagtaaaggcctagttataatgtaacagggatcattatgacatcaaccccaatttattctaggtgtcttgagtagtaaaatctcaacattttaagaccaacatgagcctccatttcatgtgatgataagatataccaactgatggagaccaacacaaatgaccttctcatccatggttttttaaaatgatggtgaatattggaattcctgaagatatgatttctatcttactcagcttagtaagcagctatcacttaacaatacaaaaccagagattatcagtagcaactaaattatttcctctctcttctgtctacacgaggaaacactcataaatgcacggggaggaggtcagaacctgaaagcctttctttggataagagcatcaactgcaggtaccacattggccctgtgatgctaatataaaaggagctaggcccaccggtaccgaaaagttacttagaaaagtgcggaggcttttaattttactttttttaaaagataagaaatagaatttacacacttggggctggcccacgtgtttctgtgtgtgtgtatgtgtgcacgcacgcgcgtgtgcgcttacagggatctctgagcctatggagagagatgtagctaggatagagtggacatctgaggtgggaggtgatactagctggcagtccaatgaaggggtagaagatggtaggcatcatgttagcaggctttctgatgctccagaattttaaagctggcctggaatctcacctccgcgatccatcattttggaacttaggaccaccattagccagtggcaaaaaaaaagttgaatgaaggaacaaacaattattgcttatgtaattcacttagcacatatatgatgttttaaattcttatatgtgtcatctatttttctttactttaaaattttgcaacagttacagacttatggaaaagtcacaagtacagttgaaacctttttttcttagtcatttgaaagtaacttctcagcaagatgccccttctcatttatttctctcttcctgtctctctctctcacacccctcagcacgtccgatgtatacttcctacaaacgaggatacaccccatacaaccacaacacaaactgtcaacatgaggaaaccagcactgatgtgtcatcaccacctaatcctcacaccccactcctctttcgcccattgccccagtgatgtctttcagaaaaaaggatctagctcagaatcatgcatgacatttgattgtgctgtttctttagtctcgttcagcctggaagagttccacagtcttttgttaacactcatggtcttgacactttgaggactgcaggctggttattttgcagaatgtcccttggtctgagcttgtctgaggtttcctcttgcccaggttgagggtgtgcatcttggcagcagtatcagcaaacagatgctgtgttctcactgcatcctatcaggtggcttctgatttcaatttgctctgttactgatgatgttcaattcggtcacttaagaaggtgtctgctgagcttcttcactgtaaaattactcttttcccctttataataaatacaaatttcaggtagaggcacttcaaagatatataaatatcctattcattatacaattttccatttattcatccatttatttatctctgtatgcagtcatggttcatgtgttaatcaatggactatgatccaagactatcattatttattttgatattcacattatccccactgtggtcagtggggggccgttgaagctggcttctgtatcgtcttgacttgggtcctcatgcccctggacctcctccatgctcaatggcacagcaagatattccaggctcatccttccattatccccattcctaccctctccccaagaagccctggttcctgccagtgggaagtggccctcagaagccaaggtctgagtgctagatatgttcattgcctctggagcaccattggtcccaggccttctcagtgatagaactagggaagatatggatgtacacacacaggtatgcacacacctctatctatagttctctatctacctatacagtgaacactatgagctctccaaaaccaactccacagggctcattctagttttttttctttccacatctgtaactcccttctccaacagtgagacgctggcttctctcactcccaactcatttatctaccggacctatacacctgaacagtgcccaactctgccaccatcccctccccatgtggatgccgtcctctccctgctccagctgcctctgctgcatgcaggtcctcctcgttctgctctggctctgataccctgcaccagatcagcctcctgtaaggatatctttctcatcccgttgaggcctccacaccccacggcaggttgccccctgaggaagcccgtctctggttcttgccctgctcctgatcaccatggctcctcccctaaccccactgttgccgtcccctttctgtgcccagtatagtggctgtaggactaaattgtttaaaaagggtatcattatttatttgagctttgtgaagccaagaactaggctttaagtttttctgaattctgaagacatgcttagaaagaagaatcaacaaaactttatgaccaaatagaaagagtgagagaccaggcagaattttgtaattgatcctttcaaaagatacaaactaaaggttcccttggcagggaggtagggcatggggtggggtaggaggactagtgacagcttaacatatgtttgccaaccaagaactgtttaaaaagcaagtcgaatcagaatcccagaccctacgagctggaggagcctggccccacccctcattttgcagagctggcagcaggtctgagaggttaagtgacttgctctcctcttctctttccgagatgaattattccgtgagtgctgggctggtggtgggcatcttcatcgggtttcagaagaaagcctacacttctccagaaaaccttcctgcccttgtggcactgctcctgctgtatgggtaagccgtttgggccattagctaatgcctctgaagagaagcctggtggtgggggtgggggatcatctcctgacagaaaacctgggctgtcctgtggtggtagcacccacaagtttagcttccggccccaggtagggtctgaagctgataaccagggatctgtctggcttctgattctgactccactgacagaggtatctctgaggcctggtcctgtcagtgacaatgagagaagtcccacatgatctgaatctcctactcaaactgaggccttgaccaaagcctgggggcagccattccccaacccctcacccagctctgactctcactcatctgtggccaatctgtccacctcagtgtccccatgtgaactggccaagagttaccgcccacagtagaagactccggccaaaaagctcctcctgagtcagggacagaggatgacacaggggttacatcagcagagttacagggcccagcatgcaactttctttcccacgtgtgtaaatttgaatgagtaattcatccatctcggcctcagtttcctcatctgtaaaagaaaatagtgatcctggtccttcctctgtgggccagtagagccttgccaaagcattgttctccacatctttctcttggaaatagagaatttgggaaccaacctgactataagctgtgaagatgagctcactgggctcatctgagatgacctcagctgggctttgctgacccaggctagagtgggaggtgttgcaggctggagaaccctcctatgaattgtacagggctttgtagtttacagagtatatacacagctagcagcccatttgctcctcacaaaaccccatgaagtggtcaaggcaggcatcattatctccatttaaagttgaggcacagagaccaacaaatggagtatctctctggtcccctgggactctggccagttcacacacatcacctcaggtgtaaggggagtgcattatatccagacgtattgtaggtggaatggaatgtggaactccatcactctgagttgtctcatttcacacagatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccgggtgagcataactttcttggcttttttgtttgattagtaggatagtagagtatgtgttggtcgagcagagccaggggcaagcatcgtacatgtagcagctgtatgcggatgagtgccactttcttcctccctacccccgaccctgcctcctttccttccttccttcctcccatccttccttcctctttccttcttctcctccctcctccctccttcccccgtccctccttccttcctttttcattgcttccttccttccttcgtccctccttcccttcctctttccttctgccctctctccctttttcctttcatcctccctccatccctccctccatccttccttctttcttccttctttccttcctataagcacctttttcatttctgtgctctgaatgaaatggttttctgtgtttattctgcaagcaaaacttgattcttgcaataaactttaagctttgcttactctttcagaaaggttttctcagggactttgggtgttgggttttacacacacacacatcaatacatttgggtaatttcaaaatctaaaaggaacaaaaaggcatacaatgaaaaaatctccttcctacccctgtttcccactcatgcagttctcttctccagaggcaaactcttacttgagtttcctgtgtgctctggagacacatcagcagatccctatacggtctttctcccgctttcttatggaaattgtaacactctgacatatactattccttgggcaagttaatcttgatgaagagactgggtgttctccatgctgaatgcctcacttttatgagctgccaagcccagttgtcccttccacctgacctccccctgtccagagacagatggccaaactgaatcataaaaagagggggaaaaaaagaaggcagtcgctgcagggctgtctttactccacactccacactcccagtccccaccgctgtgtctgagtcctggctgtggctgtccttggaacatttgcctcaccacgtgcctgtgtccccaggcgcctcaacctttcctctcctcattagctcttcccagttcagagggtgggaccggccagcacatctgcactgctgccctgccacacccacctccacctgcctctgggccccactggggaacacaggacaaatctgtgcggaggccccaccatgaaccgcccagacccgtggacccctgagactgactctttccagatcttgttagggtttcgtggctgctaggcaagtaacgaagcctcatctgtcccatgaatgataagaaattcagcatgtcagagtcagactctggaaaggcggggggataagaacacagccccagcagatggccagagcacccaggtgactgaaagtgctgctttgcagagctgtgtttgccacaggctcacagcccactaagtcttaagacagttttccttcagaataattaaatagccagcttaaagcaactcagaacattttcccctctgaggctgcacccatttagccaacatttgctaagcacccgccttcaaaaacctggtattttcatgtaaattatccgatacacagctgctatggaaacccccagtatcccacaggaagctccccagctcccagcagctgccggcccgtgtgagatcaggaggtctttaccagctgaacaccacgtgccgggtgtgtgctgatataaacaagcgtggcccactcgtcctgccctccagaggctcccgttccagtcggaaaaggacctgcccacgaagtttgcaacgatataagccacagtgtatgatcctccataatacagcgtgtgacagagcagcagaggagcgaggcagataacatgctgcaggccagaggcagcgggaagagccaggctgcaggggctgggggagccgtggtggaggaagttcaatttcagcctgtagatttctattagcccatttaataaataatgaagtgcctactctgagctaatcattgtgcaggtatttaggaaggacaaaaaaataattaggactcagtgcccaccctccaggggcccactgactagtagagaaagtaggcagatttttaaaaaattaatcatgggaatgtgataagtgctgggagagaggaatggatactttctcatgggaatcttggaaggcttgtaagggaaggcactctctgagccagctgtctaaagaagaacaggaatctttaagaaagcagaagggaaaagagcattctttcctgcttggagcaataggtaacagcctgcacatgcccaggcctagaggccaaagagcacagtgattccagaaagagtggggagaaagggtaggcagggaaggatgaggtaatgtgggcgcaggtgtggaggctggagagggaggaggttgtgggactgggaggagccagatggaatggacagcagtggcccagccaggagctatgctggcctcgtacgcctcgatgtcccttctattttctcaggggaggctctgcccaacatgccaagtccgaccacttgaaaacaagtccctggcttaacacagaccccagagagagtctccaaccctcctctccctagacaatggtagttgccctgtgaggggctgaaaagcagagctggagatggctcagggcctggtgttaacaaatgccttgagggctcctgttgtttcaaagtgagtctgcagggagagctccctaagtggacagcaggagggctgcagcttctctgcacattcctgctgtcacccccagagtcacctaggggaggggtaaggacagtaatgcaggttcctcacagttagcctcggtgcccacatggtactgagcatagtaaatgtttagaagatgctgcctggctagacaaaggggaagctcccgcccactagaaacttgcagggagccccagtccttgattggtcatttaattgattagctccttggcctggccttgaggcactgcttgtaagtacttcatgacctccattgcaaacccatgatgctctgctggacaaatccctccagtggccagtctggctgcaaggactctctgtctgcaggccttgccctgtgctgtcctgtgagagcatctgggccccacctgctgaagagagggggggtggggtttgccccgtttccaacagtcctacttctctgtttcagacgctgctcaggttcaacgccgtgctgaggaagctgctcattgtcttcccccacttctgcctgggccggggcctcattgaccttgcactgagccaggctgtgacagatgtctatgcccggtttggtgggtggtagccgaggcccatggagcatgggccctgggtccaaagctgggagggttaccgggggggctcctgcatcagactgtggcaggggctggtgctaggaggggaccttgttgggctggaggtgtcctgccagctggagaggattagggtgcctctgtttccatggctggggagccacaggagggatggagggcagcccttatgaggcgggtgtttggctcttgctcagttcccacataaggcctggtctagtgggccctgtgctgtggccaggtctgtggggtgagctggggcggctgaagtggactcaattcctgttgatgcccaggtgaggagcactctgcaaatccgttccactgggacctgattgggaagaacctgtttgccatggtggtggaaggggtggtgtacttcctcctgaccctgctggtccagcgccacttcttcctctcccaatggtacgtccatgccacaccctgggccagtgggcagctcagggcatccagaactggaccttatacccacatggtcatttctttcctcaggagccccactccacaatgttttttctacattctcaaagcctggcttttctccaataatacaagtagaggatcgggttaaaataggcacattcaaatatgtgaagagcatccactttaaaatatttaaaatgcagtgctattaatttcaattgctgatatttaatccttctcatttaattaccaaatgtgtattttgattagatgatagtattgcaaataacaatggttacagggtatccaaagtactaggaaatagactaatgtatttatgagagaaaggacacagcaggcccctttgctaattagagatttgggagcatgggagtaatatgggagccatgtggaggggtgcgggcagtgatcacgaccccccactcctggaggaaggtgggtagctgccaaccctgacttttgaccagggcttctcaaatgccaggttagctggcaattgccattcttccgcaggctcttcctgaagctgggtgggcccctgcctcactcccctctgcaatccagtcctacctttattgtcctcacccaggggcctgaattgccaagcagcagcccttcctagcaagctttccccaatagtgttttgtttcttaacttttcctcctctcaggctgagtgtggtcacctgtaaatagattccaaggacttggttttatgttttgatccacagggaattgatttattggaaatgaatctgcctttctactcacaggactgtgagaggtgaatgagatcacaggtgtcaacacacgcctgatgaaacaggatacacaagcagttctagttatgggagacagtgtcaggaattgttgtccttggcaccctcagcccctgcagaccctttctgcagccttggccataccttttagaggcttttgtgtgggagagagcaggtcaggaggttgactacccaaattgactcattagcttcaaactctgatgtcaacacatttgaatgagtcctgcctgctttagggcctaaagaggaccagagaagtacaccatagtccctggcttccagaaggtcagggagggtttcaaagaagaggctgtgtctttaagaatggggaagattccatttggtggggcaggaggaggagaacattgagggactggaaacacatgcggaggctgggagacgggaatgaccaataggactgggaaccagggggagatgccaattgctgacagaggagttagtgcaagaggtaagtgagaagggtaggtggggctggattgcagggctgtaactacagctgcagagggagggcttcaacctacagctgatggggaacaacagaaggttttgaggcatgaggtggcctgatgacaactctgttttggaaaggtggagttggcagggcagactggaggaagtgggaggctcggaggttagtaactaccccttactgagtgcttgctgtagaggaagcattttagtcctgacggtgatcccaggccctgagtctttactctgtgccaggcactgtgctgagttcatcttcagcacaatcctatgagacaggtattgttaccctcctcctcatcacatggttgaagtaggcaaggttcagagaggtccaatgcccaagatcacacatgaggaggccaggactggaacccaaggctgactctggacatgagcacctgacctctctacctaatgcctaatgcctctcctgctgggagccctttttagaatttaagtcttaaaggatggaagcccagaaggaagcagaagcaaggaagtggaagagaggtcccatggaaaggacagtgccaaggacactgtacagccagcccaatcctgaccccttttcttcatctaggattgccgagcccactaaggagcccattgttgatgaagatgatgatgtggctgaagaaagacaaagaattattactggtggaaataaaactgacatcttaaggctacatgaactaaccaaggtaagggaatgggtatgagtttggaggtgctggttagatccacagttggcatgatgttgccattttccttctatagaacaattgatatgcttatgcaagcaatttggttcccagttttatgtagggtcatcatccctgtgttataactcgtcttccaagagcatctaattccaatgtgtgttccctgctattcatctcgggcactgacacagggcctcagtgagaatcactccagctgagcatcattcccttttctgtgttctgtttctgcagagcatgggtcagcctcgagatgtctcagtactcaccacacctctgtgcctgcccatgtcaatatgtaacctcctagtgctggtagttttctcctaaaccatcctttgctctttgttccctcttcccctccttgctctcaccctgtctcagttctcagtccggtttcttcgtatcttgcagatttatccaggcacctccagcccagcagtggacaggctgtgtgtcggagttcgccctggagaggtgggtactctgcagaccacgtgtgaaaggcttccgaacatcagctcttgtgcctgcctctcctccccataaggcagagctattcaataggaacataatgccataatgcaagtcacatatgtaattttaaatcttccactagccacatgagaaaagtaaaaagaaaataggtaaaattaatttcattagtattttttattttactcaatataaccaaaatattatttcaaaatgtaattaatagaaaaccttattaatgaaatatttgacaatttctcgttgtttttaagtctttgaatctttacactcagggcccgtgtcaactgggacttagatgtgtttcaagtgcttagtagccacatatggctcgtggcctctgatggcagcccaggtctaaaattcctcccccagctcacacacacacttaccctggggcctgacattttagaccttcttgatctctagggccaggctagctctgtgttttctcctagtgctttggcctcctgggagtgaatggtgccggcaaaacaaccacattcaagatgctcactggggacaccacagtgacctcaggggatgccaccgtagcaggcaagaggtgagtatcctgctcctcctgtctcagggagtctctcacaggtcctgtgagaagaataggaagggtgatcatcagaccctatagtagggtggctctgaggccctgaaagatctgtacagagaaggaggcctcccagagagcatggcccaaaaagcccaacacatagacccaatggaaaagtgaactgaattgtgatagttaagagattcctctgttgggatggattcttggaaagacctgggaagcactaagtgtgtggttcttaatctcttagaggtcacggaaccttttaagcatctgatgaatatttgtagcctattcctataaaaatgcaccattgcttcccattacctccctccacacatttttacaaaacgtttcagggagtttactgagccccaggtcacatttatgatcctgcaggagctcttgaatcccaggttaagaacccctgtgatgaatgaagaatccttcctctgggttgagtttctagataggggctcatgcatgggcctttggggtagcctaacctgcattggctatttgtaggctgatatttggctttgccagaccaaggagcatagagggaaaactggcgtgtgcccttggattctggagggtgactgctgctctctgtaataaaatgtgtttaaacagactggtcccctatgggcaggacagagaggatgagctctcactcatctgcctctttcctggctgcaggaaaagcttgaacagtaaaacttcagcacacacaatagaggtgcccagaggaagcctctgccctggtttataagtggagttaggtgctgctgacatctgtccagcatctgcttgactggggcctcttcctctctcctgaaagccatcctcagcatggcccaatgcccagtgggcaggacgagtcctgagcacgcttcactggctcagacaggatgaatttgattctttggcctccatagccagccctactgggtttacagaaaagggacaggcaggggtgaagccaggtcatggctgagtccatctcaacagatccagcttcacctgcaagtgaccacgcaggtgacttcctcatggtgacaaaaggagtcatggcagggtagagatatcataccatggcaggggaaagatatcatagaattttccatgagcacatttatgagacatcaagttacaactgtgtccaagtgaggcacagtctgacatccagaaggtaaaactgagctggacgctagaaagaaactataggcttaagacacagaattgggattatatggtagggtagctcccactaatttggaaacgtaccctacttgcttccctgagtagttttaattggcccagccatgcctttggtggcttttgtcattgtggggaactgtaatggtctctctgtaccatcctatatcatccatcctttattcatagaccctaagctataagaagaaaaggatgagattagactaaatgtctatgtatagtttattttccatcttggcaatatattttttagtgggggtgaatatattagccaaagggagttggtggaacccaactcactctacccctgctccctgcaggcctctcgctgtgggtagttatctgactggctcctctttcattgctatctttgccaataaatacagatagagaagtttacttccatcgggacacatgcatcttttctagttacttcccaaatgtctgaaaattattgataaatcatgaatcattttcttaaacctgatcttccctctgtttttaaactcacatgtgaggtgatctgatccaaaatgaaagctgacttttggcgtaacagggattcaattaatcctagacatggaaacatggaagaatctgacaggattcagtttctaaccgaagggcccctgttttgattcccaaatatcccatgcatttctgaagccaaataggagaagagaagaagcagcttccttttcccgttggcagaagcttctccagccctagctctatggtcatccctccactccttgaaggatactcagtaattgctttttttcttgcagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggtgaaaaatgttttgttgtggccacataggagtctggttaattacaagcctgtttcatgagagtgcattctcttggagatgagaaactgaagcgtgctattcattcattcattccaacaaatgtttactatgtgtctactgtgtgccaagtactgttctagaaaccaggagtatagcagtgaacaagacagacaaaaaaaaatccccactctcatatctaacaaaatgttgtatgcatttatcctctgactcagcaatcacacgtctaagagtttatcctgaagatgcatctcccacagtgcaaaatgaatatgtataaggtgatccattgcatttgtaattgcaaaatgctggaagttacctaaatgtttagtcattgtagattggctgaataatttatggtacagacacacaataaagtcttacgcaactataaaaaagaagaagaaaagtctcagtaaactgatatggagatatttccagtaaatactgttaaatgataaaaagcaaagtggaaaacagaacatagagaacgctactttgtatgtaagaaagaaggaaaaacaagaaagtaaacgtatgtctgcttacctttgcaaatagaacgtagaaaggataaaccagaaaacaatgaatttggtgatcaacaagaagaaaatgggaagaaagaaaaatgggaggaaacagtacttctggggatatatttttgtatagttttaatttttggaagcatgttaatgttccacatattcaaaaaaaatcagtaagaatgggaagtaggcaaaaatgaaaacaaaaagaaaacctaacactgacagcaaactaaataaagtaacccaattttatttcaaataaatatcataatcttgcaaaagggggatagagctaacacaaacaactgctgaacacagtgtttgactctatatcctcattcttgggcagggtggagcgggggagaagaactacaaataatttctgagttctttttagtttgttttttatagtggtataggcaaagtgattctgaaaattttagatgtgttacaggattaaataaattaataaatgttttgatgttattgggacccagaattctcaccgtggaagaagggacttacaaatatggaaaagggaaaagcaagaaagaactgtgaggtcatggataggaaccggaggtagcactgggaattcaggaatatttatatgcttgtgtttgtgggtgcatgcagatgtgttcatgtttcatgcacataggcatgtatatatagacatatatttgcatgtgtgtatctgtcttccgaaaggctcaagaagcaaaaacaccccagtagccatgagcacacttagcactcaggcttttgtcttaataacattccccactaaaagtaaccctgattcctccaataaatgataagttccagggctggaatggcataggtataaaatgaacctggaatatcttatgccagaaagtaaggaagtgcttttaaaaaaaaaataaggggctgggcatggtggctcacacctgtaatcgcagcactttgggaggccaaggtaggaagatcgcttgagcccaggagttccagattagcctgtgcaacatagggagaccctgtctctacaaaaaattagcaaacaaattagctgggcctggtggtgcacgcctatagtcccagctactcaggtggctgaggtgggaggaatgcttgagcccaggaggttgaggctgcagtgagctgtgatcaagccactgctctccagcctgggaaacagagcaagactctgtctcttaaaataataataatataattttaaagaaataaaagtaactctgtacagattgcttattggttacatgggagaaacataataattttacaatggagaaattagacagcaccttaactgggtgatcaaaattaaccataaggggcagatggacatctcatgccccgagatgtgataccctgtgaaggacacaatttcacttatgtagaatccagattggagatatgtaacctgaatcttatcatgaggaaacatctgacaagctccaaagaaggaatattccttaaaaaaaaaaaaaggagactgtattcttcaaaaacataagagtcataaaagacaaagaaagagctatggaaatatctctgatcgcaggaggctaaacaggcataatgactgaatagcagacaatagactacatcttgtgcagaagagaaaaaaaatgatagaaggatattattggaccaactgacaaaactgaactatgaacagtagattaggtaaatgtatcataacattaagtttactgacattgataatgtactgtggttatgtaagagaagatctctattcttaggaaatatgccctgaagtatttaggagtgaagggctgtgatgagtaatttaccctcaaatgggtcacaaaaaattgtgtgtgagagagagaagggttttattagttaataattctatgaactatttttattcctatatgtttgtgtgagtttgaaactatttccaaataaaaagttaaaaatggagattacattctagtgggagggatagacgatctgtagataaataggtaaaatatccagtacattagagagtgaaaagtcctcagggaaaagtaacgcagggaggaactgctggggcagggtttgcattttgaggtagggtggcccagggagagcctgcagaggagagaacctgaatgaagaactagaggtgagagaaggagccacgtgcacacctagggaggaacattccaggcacgggggactagtatagaaggcagaagcatggtgagcttgtctccagtggcttccctagatcccctcctgcgcatgtgcacacacacctggtgtctctgtcatcgttccctcacagcactgtcacgatctgccagtattctgtttattttgactgccacctccccgcagtctgaggatagcagcaatggctgtgttcacattgttctccagtgcctggttcagtgcctggcgtatggtcagtgctccataggtatgtgtcggatgcacaaggctttgggtgtaaccctcttgacgggtgggatcaacaggtctgggactcaccatcttctcaaacagagccttcctcctccactgctagccatggtccaggacgctgggcgagacccactgtcttgctctttgtaaggctgaagtccatttcccaggcggctacacccaacagatgctgagcaggctgggccaccctgggatccaagacacagagagaaagagcccctgtctggcgcctgaagcacatgccagaggacaggagccagcaggagcctgtttcagcctagctggggatttcattctggaggcgtgagatctgggagcccaaggctttgaactgggggaggtttggggtgtttgcttgtcttctccaaatggcatttctttctcttccctaggttgcaaactggagtattaagagcctgggcctgactgtctacgccgactgcctggctggcacgtacagtgggggcaacaagcggaaactctccacagccatcgcactcattggctgcccaccgctggtgctgctggtaactgcgggcttgggccgcaccaagggcttaaaccaagtgctgggtctcttgggttggggaaataggttctgggtcggcagatttagaaactgcagcagtttggctttagtctggactgtttcctgtgttgctcattttgagcgatcagcccagtgtttggttcacacagctccggagaaaaacaagtcacggcacagccttgacttgggactgcgcacatcctgcgttcccaggatgtctcctgtggggccatcggctcacagccgggaagttcagcccactctgcggcctgtcggtgtctggtccccatacaggagcactgagctgggtcaaaggctcctgagctgagccaggccaggcctgaggccatgcccacgcagcccaaggatcatgagggcacaggacatagcgggaaccaaggaagtgacctgagtgacctccctgccttctgacaaatgtatttgcaggattttctttttttgaggagaattctgtcattgccttaatccactttaatcccctcgtgggctgaaatgggcccaggatggacgccacgcttctttactcttggatccacctcctgccttccctaccctacaccagggtacccctgtcttgctcaagtgaggggagtgactgtgtgcgccttctgtcagctcatcctccacaggggagccagcccagggggaagcagtaatcagaagggccagctcccagcctgtgcccccaaccttctctccaccccccaggatgagcccaccacagggatggacccccaggcacgccgcatgctgtggaacgtcatcgtgagcatcatcagagaagggagggctgtggtcctcacatcccacaggcaagagattcccagggctggggaaggtgggtgggaatcctctcctgctcacctcctctctcctgccccacagcatggaagaatgtgaggcactgtgtacccggctggccatcatggtaaagggcgcctttcgatgtatgggcaccattcagcatctcaagtccaagtaagcagatggtggggcgtgccccttgttgccttctgtggatccacctggatcctgtgttctccattgacacttggaagagtcctgctgctccgtcatcccctggggcagaggcaggtggtggctgggcctcattctccagcagcagatggagaaggccatcatgctgataagaaactcctctatattggcctaatttcctgtggtcgaagactcgcccaagtctctggatggggcatctgatcaggatgcatgcagagcctggctgggatgagggagggctgctaccactgcctcaatatttcaccacttatctcaacagatccgggacctgtggcctatttactaagagtccactccaatgtaggaatggttaggagaccaactgacttgaggacccatctttgtttttagaatattgtatgcttttgagtttgaaaaaagaccatatgttatatgacaaaccaacaatggcagtaatcttgaataggattatccttatcctgtacccacacattgtaaactattgtagataattccttattattaagagtttgcatgccaaagctaacagtttaagattatcagcatattgccgtgctcattcacgttctgatatgctttataacctagaaaagagcagagttacaattactcatttatttaacaaacacttattaagagctcagaatataagtcactaagctggttggtgggaggaacagcacataacccaccttatctatgctgaggtgcataatcctgatgcacccacaggagggtgttacacagaagatgtcatcctttcatatgtgtcagagcagataaataattgagagaaaggtctaatagattagctgcttgtggcaagtggacgtttgacccatgatttattgagcaactacaacttggacactgcatagatatctatagaaatagcagcatgtcaggtcaccagacctgtgtcagcaacttcctgtgtccaactgctggagaaagggaagtctcctattcctttccctccagctccttaatatctccatgatagagggggtgagaggggagtgttccctgtgtggagggatggtgagttttctggagctgaaaggtaaacagcctttctcctctgcatcttactgcagaggagaacagccctagactgtggaggaagctttggagtcagttatgactgacacaggataccagggcatagggtactgacacccgctagccgtgcacacactctctggtggaccatcactcatccaagagagggtaaccagccatcctgctgaaggagaaagaaagcaccaatggcccaagccctagcagctccattgtttcaggaagcttcctcagggaagtgctgccttcccgagcctttgctcccacctggcccatcagcccttaccaccactcagtatgcactggtccacgtgtctttatgggcagtcttgggatccccacactgggctaaaactacctttgacggccaggtgcagtggcttacacctgtaatcctatcactttgggaagctgaggcaggtggatcacttgaggtcaggagttcgagaccagcctggccaacacggtgaaaccctgtctctactaaaaatacaaaaattagatgggcatggtggtatgcacctgtaatcccacctactcgggaaactgaggcacaagaattgcttgaactcagaaggcagaggttgcagtgaatcgagatcacaccactgcactccagcctgggtgaaacagcaagactctgtctcaaaaaataaaataggctgggcgtggtggctcatgcctgtaatcccagcactttgggaggccaaggcgggcggatcacttgaggtcaggagtttaagaccagcctggccaacatagtgaaaccctgtctctactaaaaatacaaaaaaaaaaaaaaaaattagccgagtgtggtggcaggtgcctgtagttccagcctctcaggagactgaggcaggagaattgcttgaacccaggaggcggaggttgcagtgagccaagatcatgccactgtactccagcctgggcaacggtgagactgtctcaaataaaataaaataaaataaaataaaataaaataaaataaaataaataaaataaaataaataaaactacctttgacttcagcaagtacgattatcccacattaccatgcagacatttgatctctaaaaactggtatcaaatgatttctccagggactaccatggtttttctctcctagttttcagtatgtacacaggtctatggtatgggcctttaatccccagtatttctttttttgttgttcttgtttgggtttgtttcttgtttttcggtttttttgagacagggtctcactctgtcacccaggctggagtgcagtggcatgatcatggctcactgtagccttgacctcctatgctcaagtgatcctcccgcctcagcctcccaagtagctgggaccacaggcatgtgccaccatgccctgctaattttcgtagagacagggtctttcttgttgcccaggcttatcttacattcctgagctcaagtgatcctcccacctctacctcccaaattgctgggatttcaggtgtgagccaccaagctgagcttaatccccaaaatttctgatgagtctactccttattttgggattaccttaggcccaaccactaacagaggcctgtcctgcactgtgtgcatcccctagatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggcaagttggccctggggcaccgagagctgagcaaagactggtccagaacacccagtgtgggttggaattgccataagagggaggcataacattcccgatttttaacaaactcttgccctctgtttattggggtaaaagctgatatatcagaaattgttttctaacaatattttttagtcatcaggaaacttcattgattcttttttttacattttccttccctgtgatgctatggtgtgttatttcattcttgctcgtttgtggtggtggtttttccttcaaatcagctttattgatgtgtaattaacatacgatgaaacacaggttctttgggaggccaaggcaggaggatcacttgagcccaggagtttaagacaggcccatgtaacaaagtgagactttgtctctacagaaaaaaaaaaaaaaaatcagaaaattagccaggcgtggtggtgcatgcctgtggtcccatctacatgggaggttgaggaaggaagattgctggagcccaggaggtcaaggctgcaatgagctgtgttcataccactgcactctagtctgggtgacagagcaagcccctgtctcaaaaaagcaaaacaaaacaaaaacacctattttaaatgtacagtttagtgagttttgataaacgtgcattccatgtgtggtttttaaaaatgtaatcacattttttattgcggtaaaatataataacataaaattgaccatgccaaccatgtttaagtgcacagtgcagtggcactaagtacatttacattgttgtgcaaccgttaccaccatccccgatagaactctttcatcttgcttcagtgaaaatctgtgcccattaaacactaactcaccacttactgcccccctcgcccttggcaactactgttctactttctgtctctaaggctctgactactatagatacctcatataagtggaatcatacagtgtttgtccttttgtgtctggcttattatgcgaggacttagcataatgtcctcaaggttcatccgtgttgtatcatgtgccagaatttccttcctttttcaggccgaataatattcctttgtacgtatatgtgctacattttgttcatccatctattcattcattgatagacatttgggttgtttctgggttttgtgtttttatatatgtttttttaaaaataaacatctttagagacagttcagtaaagcagtggaaacagggaagtctccatttaacccctgaggatctggctcacctgcaccttctcatcagcattaagcagagggaggcacgagcaggagccacctgcacactcaatgaggagctgaacagggatcaattacctttttttttagttattaggatgctgctagctgagaatctgccttgccttgattaccccaatgtctggtgcccaagtcccttgagtcctccagcaggaactcctgtggcatcactcaggagtctagtctaagaagctagctctgaccagggcagtggtggccaggcttctgtgagtgggccagcctcccccgggtaggacacaagccataccagcagggctgtatgtgaactgtggaaaatagagagcaaagtgggtaggtgggtgtagggtgctgttttcctggaaatatctacctaatctcgctcttctcttacctctaggtgtttgtaaattttgctaaacagcagactgaaagtcatgacctccctctgcaccctcgagctgctggagccagtcgacaagcccaggtacccctgctgcttatgcagtccacagcttgaggcagttccttggctcagagcccagctggttcactgggcttgagttgctccaaggctcagatatgcctcctacagagagccccacccacaccacggtccctaccaagtccccaccacatcctcatcacatccttgctaagtccctgccactgtgtgttctgtgctgaagaacttttcattcagtagttgtaggggttcctattgtaatcaggaaaccatctggatagcatgggagagcatttttgaaaagaactttcccatgtttttgcttacagcaaaaaagcttggatttggggaataaggagcagagaaggtaatagagaatattagaatgttttgggtgcttgacatctatgtctggacatgtgtttgagtttcaagggaagggacttaactggcacatcatttcagtgtcagacacatttggttagatcaaggaatagcatctgttgtaggaagagggctctttgttctttataaaaattacaagaagatggagaaagaagcaataggaggtatgtctcctggcttgtgataactcttggaataggtgcttgtaggttcctgccctggcacagtgccccatgtaaggagcacaccacccaagaaggagagagctagagcaagtactggaggaggcaccagcatcccaatgccttggcttaagcctgggattgtagagggatgaattagccactctcttctgacttacctggagagtaaatcaaatcaaatcaagaagcaaggatatgcaaaaaccttatttccccataaagtttttattctgcccagtttctggattgcaagaaaaaccaaatacagctaatgattgaaacactgctgtctaaagcagtgcttgtgatgaattttttcccttcctcttgaccagcagagacctaatggctacttggcaaaactgactttgtcttcccaccccttacctgccagagggcccagaaatgcctaaggctcctttagttacagaaagtttgcttttactgagatcttccagccactgattcccatttatagatctggtgattgctgttgacatcagttgaaaattatttttaaaaaccacttgcagttgcaaatcctttttataactctgtaactcagaatatagaattgggtagcaaaattgtttcccagaattaccaatggtctccccacccctgcctggcatgttccctcttaaaggactaatcccaccacatcacctctgggccaggcagaacatcaggggtgctgatgttctgtgatctacagcagttaattccaaacttttctcccttattggatgagatcatttttctattgtgttttttacatttttgttcacaaagattagaaaacctgcaacacacttattggcatatttttctgataattttcatccaaaacctaattctgactttacaacatactatctttacaaaggtttgcaaaaattctttcatatagcattgtatatgtctgtcatgaaataatagtaagtatattattgtttacattataccacttcaaaataatttcctttaaagtattcttcaaacaagaaaaaggcaatttctctcaagaagttttagagagaatttacaacttgctcctaagcaaatgtgagaacttcaggaggttcatctggccattggctttacaactccaaattgtgagccaggaccacacagatatttctctagaaatcagcgtttgcttaccaagaacatttttactctccaaaggactccatcctggaaaacatgttttgggataaggtcttatgcaatcttatactctgttattaaaaccagtgagggtcaaggtgttaatagattaagtagtgacagatgatcagacaacttagaaacatcctaaataggttaataattatgtgaccatcgcatgtgcattcccaaattaggaacaactcagatcaatttctaatccttattcttacactgttccagttcccccatataactcgtatctttgtgttagtttcagaagtttctgaagtaccctcagccttgatggggatcctcgcaccacctcaaatcctgttctcagccctaagaactgtgttagtcatcctcttaagaggatgtgtgattttaaatcagataatgggataaaccacatttcgtctagactggtcaggcctttgtccagtcccctcctcgcccacactaccccagctccacagcgggcattggttcaggaattcaacccacactttataactggagacagtatctctccagttaaaaaggtcaccttggtgtccgcttctcaaggaacatggacatctttattaatcaaagcccaagctttgatctggagcctaatatcctgcactccagctctcatctctcccctcccccagtcacactttcatgcttcccagagccacccctacaggaagtggtcaagggaattctatacctcagggctgacctaaattaggatttcttggcttttaagataatggtaactttcttaagctaaaaaagccccaaaagaccctgtaagagcccttggaaacagcaccatgggtgtagcttccccccaggatgtaagcatgtatgcacacatctcgtatgtgtgtctttgtaacaaatgcctggatcttagtaccagggagacctgattcatagatttcatagagaaggagagaaagatggcccataacctgggtgatctgacagaatcacagtgccctcagctgagtgcccttcagaaattgattgacaactgtttagcttttgaaatctaaaagtagtacagcatctcagaaaaccaagatgacgcgagtccatgtgatctccttccacaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaa

SEQ ID NO: 2 - RNA, 7326 nt, Homo sapiens
aggacacagcguccggagccagaggcgcucuuaacggcguuuauguccuuugcugucugaggggccucagcucugaccaaucuggucuucguguggucauuagcaugggcuucgugagacagauacagcuuuugcucuggaagaacuggacccugcggaaaaggcaaaagauucgcuuugugguggaacucguguggccuuuaucuuuauuucuggucuugaucugguuaaggaaugccaacccgcucuacagccaucaugaaugccauuuccccaacaaggcgaugcccucagcaggaaugcugccguggcuccaggggaucuucugcaaugugaacaaucccuguuuucaaagccccaccccaggagaaucuccuggaauugugucaaacuauaacaacuccaucuuggcaaggguauaucgagauuuucaagaacuccucaugaaugcaccagagagccagcaccuuggccguauuuggacagagcuacacaucuugucccaauucauggacacccuccggacucacccggagagaauugcaggaagaggaauacgaauaagggauaucuugaaagaugaagaaacacugacacuauuucucauuaaaaacaucggccugucugacucaguggucuaccuucugaucaacucucaaguccguccagagcaguucgcucauggagucccggaccuggcgcugaaggacaucgccugcagcgaggcccuccuggagcgcuucaucaucuucagccagagacgcggggcaaagacggugcgcuaugcccugugcucccucucccagggcacccuacaguggauagaagacacucuguaugccaacguggacuucuucaagcucuuccgugugcuucccacacuccuagacagccguucucaagguaucaaucugagaucuuggggaggaauauuaucugauaugucaccaagaauucaagaguuuauccaucggccgaguaugcaggacuugcugugggugaccaggccccucaugcagaauggugguccagagaccuuuacaaagcugaugggcauccugucugaccuccuguguggcuaccccgagggagguggcucucgggugcucuccuucaacugguaugaagacaauaacuauaaggccuuucuggggauugacuccacaaggaaggauccuaucuauucuuaugacagaagaacaacauccuuuuguaaugcauugauccagagccuggagucaaauccuuuaaccaaaaucgcuuggagggcggcaaagccuuugcugaugggaaaaauccuguacacuccugauucaccugcagcacgaaggauacugaagaaugccaacucaacuuuugaagaacuggaacacguuaggaaguuggucaaagccugggaagaaguagggccccagaucugguacuucuuugacaacagcacacagaugaacaugaucagagauacccuggggaacccaacaguaaaagacuuuuugaauaggcagcuuggugaagaagguauuacugcugaagccauccuaaacuuccucuacaagggcccucgggaaagccaggcugacgacauggccaacuucgacuggagggacauauuuaacaucacugaucgcacccuccgccuggucaaucaauaccuggagugcuugguccuggauaaguuugaaagcuacaaugaugaaacucagcucacccaacgugcccucucucuacuggaggaaaacauguucugggccggagugguauucccugacauguaucccuggaccagcucucuaccaccccacgugaaguauaagauccgaauggacauagacgugguggagaaaaccaauaagauuaaagacagguauugggauucuggucccagagcugaucccguggaagauuuccgguacaucuggggcggguuugccuaucugcaggacaugguugaacaggggaucacaaggagccaggugcaggcggaggcuccaguuggaaucuaccuccagcagaugcccuaccccugcuucguggacgauucuuucaugaucauccugaaccgcuguuucccuaucuucauggugcuggcauggaucuacucugucuccaugacugugaagagcaucgucuuggagaaggaguugcgacugaaggagaccuugaaaaaucagggugucuccaaugcagugauuugguguaccugguuccuggacagcuucuccaucaugucgaugagcaucuuccuccugacgauauucaucaugcauggaagaauccuacauuacagcgacccauucauccucuuccuguucuuguuggcuuucuccacugccaccaucaugcugugcuuucugcucagcaccuucuucuccaaggccagucuggcagcagccuguaguggugucaucuauuucacccucuaccugccacacauccugugcuucgccuggcaggaccgcaugaccgcugagcugaagaaggcugugagcuuacugucuccgguggcauuuggauuuggcacugaguaccugguucgcuuugaagagcaaggccuggggcugcaguggagcaacaucgggaacagucccacggaaggggacgaauucagcuuccugcuguccaugcagaugaugcuccuugaugcugcugucuauggcuuacucgcuugguaccuugaucagguguuuccaggagacuauggaaccccacuuccuugguacuuucuucuacaagagucguauuggcuuggcggugaaggguguucaaccagagaagaaagagcccuggaaaagaccgagccccuaacagaggaaacggaggauccagagcacccagaaggaauacacgacuccuucuuugaacgugagcauccaggguggguuccugggguaugcgugaagaaucugguaaagauuuuugagcccuguggccggccagcuguggaccgucugaacaucaccuucuacgagaaccagaucaccgcauuccugggccacaauggagcugggaaaaccaccaccuuguccauccugacgggucuguugccaccaaccucugggacugugcucguugggggaagggacauugaaaccagccuggaugcaguccggcagagccuuggcauguguccacagcacaacauccuguuccaccaccucacgguggcugagcacaugcuguucuaugcccagcugaaaggaaagucccaggaggaggcccagcuggagauggaagccauguuggaggacacaggccuccaccacaagcggaaugaagaggcucaggaccuaucagguggcaugcagagaaagcugucgguugccauugccuuugugggagaugccaagguggugauucuggacgaacccaccucugggguggacccuuacucgagacgcucaaucugggaucugcuccugaaguaucgcucaggcagaaccaucaucauguccacucaccacauggacgaggccgaccuccuuggggaccgcauugccaucauugcccagggaaggcucuacugcucaggcaccccacucuuccugaagaacugcuuuggcacaggcuuguacuuaaccuuggugcgcaagaugaaaaacauccagagccaaaggaaaggcagugaggggaccugcagcugcucgucuaaggguuucuccaccacguguccagcccacgucgaugaccuaacuccagaacaaguccuggauggggauguaaaugagcugauggauguaguucuccaccauguuccagaggcaaagcugguggagugcauuggucaagaacuuaucuuccuucuuccaaauaagaacuucaagcacagagcauaugccagccuuuucagagagcuggaggagacgcuggcugaccuuggucucagcaguuuuggaauuucugacacuccccuggaagagauuuuucugaaggucacggaggauucugauucaggaccucuguuugcggguggcgcucagcagaaaagagaaaacgucaacccccgacaccccugcuugggucccagagagaaggcuggacagacaccccaggacuccaaugucugcuccccaggggcgccggcugcucacccagagggccagccucccccagagccagagugcccaggcccgcagcucaacacggggacacagcugguccuccagcaugugcaggcgcugcuggucaagagauuccaacacaccauccgcagccacaaggacuuccuggcgcagaucgugcucccggcuaccuuuguguuuuuggcucugaugcuuucuauuguuaucccuccuuuuggcgaauaccccgcuuugacccuucaccccuggauauaugggcagcaguacaccuucuucagcauggaugaaccaggcagugagcaguucacgguacuugcagacguccuccugaauaagccaggcuuuggcaaccgcugccugaaggaaggguggcuuccggaguaccccuguggcaacucaacacccuggaagacuccuucuguguccccaaacaucacccagcuguuccagaagcagaaauggacacaggucaacccuucaccauccugcaggugcagcaccagggagaagcucaccaugcugccagagugccccgagggugccgggggccucccgcccccccagagaacacagcgcagcacggaaauucuacaagaccugacggacaggaacaucuccgacuucuugguaaaaacguauccugcucuuauaagaagcagcuuaaagagcaaauucugggucaaugaacagagguauggaggaauuuccauuggaggaaagcucccagucguccccaucacgggggaagcacuuguuggguuuuuaagcgaccuuggccggaucaugaaugugagcgggggcccuaucacuagagaggccucuaaagaaauaccugauuuccuuaaacaucuagaaacugaagacaacauuaaggugugguuuaauaacaaaggcuggcaugcccuggucagcuuucucaauguggcccacaacgccaucuuacgggccagccugccuaaggacaggagccccgaggaguauggaaucaccgucauuagccaaccccugaaccugaccaaggagcagcucucagagauuacagugcugaccacuucaguggaugcugugguugccaucugcgugauuuucuccauguccuucgucccagccagcuuuguccuuuauuugauccaggagcgggugaacaaauccaagcaccuccaguuuaucaguggagugagccccaccaccuacugggugaccaacuuccucugggacaucaugaauuauuccgugagugcugggcugguggugggcaucuucaucggguuucagaagaaagccuacacuucuccagaaaaccuuccugcccuuguggcacugcuccugcuguauggaugggcggucauucccaugauguacccagcauccuuccuguuugauguccccagcacagccuauguggcuuuaucuugugcuaaucuguucaucggcaucaacagcagugcuauuaccuucaucuuggaauuauuugagaauaaccggacgcugcucagguucaacgccgugcugaggaagcugcucauugucuucccccacuucugccugggccggggccucauugaccuugcacugagccaggcugugacagaugucuaugcccgguuuggugaggagcacucugcaaauccguuccacugggaccugauugggaagaaccuguuugccauggugguggaaggggugguguacuuccuccugacccugcugguccagcgccacuucuuccucucccaauggauugccgagcccacuaaggagcccauuguugaugaagaugaugauguggcugaagaaagacaaagaauuauuacugguggaaauaaaacugacaucuuaaggcuacaugaacuaaccaagauuuauccaggcaccuccagcccagcaguggacaggcugugugucggaguucgcccuggagagugcuuuggccuccugggagugaauggugccggcaaaacaaccacauucaagaugcucacuggggacaccacagugaccucaggggaugccaccguagcaggcaagaguauuuuaaccaauauuucugaaguccaucaaaauaugggcuacuguccucaguuugaugcaauugaugagcugcucacaggacgagaacaucuuuaccuuuaugcccggcuucgagguguaccagcagaagaaaucgaaaagguugcaaacuggaguauuaagagccugggccugacugucuacgccgacugccuggcuggcacguacagugggggcaacaagcggaaacucuccacagccaucgcacucauuggcugcccaccgcuggugcugcuggaugagcccaccacagggauggacccccaggcacgccgcaugcuguggaacgucaucgugagcaucaucagagaagggagggcugugguccucacaucccacagcauggaagaaugugaggcacuguguacccggcuggccaucaugguaaagggcgccuuucgauguaugggcaccauucagcaucucaaguccaaauuuggagauggcuauaucgucacaaugaagaucaaauccccgaaggacgaccugcuuccugaccugaacccuguggagcaguucuuccaggggaacuucccaggcagugugcagagggagaggcacuacaacaugcuccaguuccaggucuccuccuccucccuggcgaggaucuuccagcuccuccucucccacaaggacagccugcucaucgaggaguacucagucacacagaccacacuggaccagguguuuguaaauuuugcuaaacagcagacugaaagucaugaccucccucugcacccucgagcugcuggagccagucgacaagcccaggacugaucuuucacaccgcucguuccugcagccagaaaggaacucugggcagcuggaggcgcaggagccugugcccauauggucauccaaauggacuggccagcguaaaugaccccacugcagcagaaaacaaacacacgaggagcaugcagcgaauucagaaagaggucuuucagaaggaaaccgaaacugacuugcucaccuggaacaccugauggugaaaccaaacaaauacaaaauccuucuccagaccccagaacuagaaaccccgggccaucccacuagcagcuuuggccuccauauugcucucauuucaagcagaucugcuuuucugcauguuugucugugugucugcguugugugugauuuucauggaaaaauaaaaugcaaaugcacucaucacaaa

SEQ ID NO: 3 - protein, 2273 aa, Homo sapiens [sequence omitted]

SEQ ID NO: 4 - DNA, 283 bp, Homo sapiens
ttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtg

SEQ ID NO: 5 - DNA, 257 bp, Homo sapiens
ttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcct

SEQ ID NO: 6 - RNA, 20 nt, Artificial Sequence
  AON1
accccaggaaucaccuugca

SEQ ID NO: 7 - RNA, 20 nt, Artificial Sequence
  AON 1 target
ugcaaggugauuccuggggu

SEQ ID NO: 8 - RNA, 30 nt, Artificial Sequence
  AON1 target +5
ccaauugcaaggugauuccugggguagcag

SEQ ID NO: 9 - RNA, 40 nt, Artificial Sequence
  AON1 target +10
gcucgccaauugcaaggugauuccugggguagcagagccu

SEQ ID NO: 10 - RNA, 20 nt, Artificial Sequence
  AON2
gcucugcuaccccaggaauc

SEQ ID NO: 11 - RNA, 20 nt, Artificial Sequence
  AON2 target
gauuccugggguagcagagc

SEQ ID NO: 12 - RNA, 30 nt, Artificial Sequence
  AON2 target +5
aaggugauuccugggguagcagagccucac

SEQ ID NO: 13 - RNA, 40 nt, Artificial Sequence
  AON2 target +10
auugcaaggugauuccugggguagcagagccucacgaauu

SEQ ID NO: 14 - RNA, 19 nt, Artificial Sequence
  AON3
caauuggcgagcagccaaa

SEQ ID NO: 15 - RNA, 19 nt, Artificial Sequence
  AON3 target
uuuggcugcucgccaauug

SEQ ID NO: 16 - RNA, 29 nt, Artificial Sequence
  AON3 target +5
agggguuuggcugcucgccaauugcaagg

SEQ ID NO: 17 - RNA, 39 nt, Artificial Sequence
  AON3 target +10
aagggagggguuuggcugcucgccaauugcaaggugauu

SEQ ID NO: 18 - RNA, 20 nt, Artificial Sequence
  AON4
aucaccuugcaauuggcgag

SEQ ID NO: 19 - RNA, 20 nt, Artificial Sequence
  CUCGCCAAUUGCAAGGUGAU
cucgccaauugcaaggugau

SEQ ID NO: 20 - RNA, 30 nt, Artificial Sequence
  AON4 target +5
ggcugcucgccaauugcaaggugauuccug

SEQ ID NO: 21 - RNA, 40 nt, Artificial Sequence
  AON4 target +10
gguuuggcugcucgccaauugcaaggugauuccuggggua

SEQ ID NO: 22 - RNA, 20 nt, Artificial Sequence
  AON5
gagcagccaaaccccucccu

SEQ ID NO: 23 - RNA, 20 nt, Artificial Sequence
  AON5 target
agggagggguuuggcugcuc

SEQ ID NO: 24 - RNA, 30 nt, Artificial Sequence
  AON5 target +5
uuguaagggagggguuuggcugcucgccaa

SEQ ID NO: 25 - RNA, 40 nt, Artificial Sequence
  AON5 target +10
ccguguuguaagggagggguuuggcugcucgccaauugca

SEQ ID NO: 26 - RNA, 20 nt, Artificial Sequence
  AON6
uuggcgagcagccaaacccc

SEQ ID NO: 27 - RNA, 20 nt, Artificial Sequence
  AON6 target
gggguuuggcugcucgccaa

SEQ ID NO: 28 - RNA, 30 nt, Artificial Sequence
  AON6 target +5
agggagggguuuggcugcucgccaauugca

SEQ ID NO: 29 - RNA, 40 nt, Artificial Sequence
  AON6 target +10
uuguaagggagggguuuggcugcucgccaauugcaaggug

SEQ ID NO: 30 - RNA, 20 nt, Artificial Sequence
  AON7
cuugcaauuggcgagcagcc

SEQ ID NO: 31 - RNA, 20 nt, Artificial Sequence
  AON7 target
ggcugcucgccaauugcaag

SEQ ID NO: 32 - RNA, 30 nt, Artificial Sequence
  AON7 target +5
gguuuggcugcucgccaauugcaaggugau

SEQ ID NO: 33 - RNA, 40 nt, Artificial Sequence
  AON7 target +10
ggagggguuuggcugcucgccaauugcaaggugauuccug

SEQ ID NO: 34 - RNA, 21 nt, Artificial Sequence
  AON8
ggaaucaccuugcaauuggcg

SEQ ID NO: 35 - RNA, 21 nt, Artificial Sequence
  AON8 target
cgccaauugcaaggugauucc

SEQ ID NO: 36 - RNA, 31 nt, Artificial Sequence
  AON8 target +5
cugcucgccaauugcaaggugauuccugggg

SEQ ID NO: 37 - RNA, 41 nt, Artificial Sequence
  AON8 target +10
uuuggcugcucgccaauugcaaggugauuccugggguagca

SEQ ID NO: 38 - DNA, 19 bp, Artificial Sequence
  primer
cacccggagagaattgcag

SEQ ID NO: 39 - DNA, 19 bp, Artificial Sequence
  primer
cctgcatactcggccgatg

SEQ ID NO: 40 - DNA, 24 bp, Artificial Sequence
  primer
ggaatacgaataagggatatcttg

SEQ ID NO: 41 - DNA, 24 bp, Artificial Sequence
  primer
cttgaattcttggtgacatatcag

SEQ ID NO: 42 - DNA, 59 bp, Homo sapiens
agctcttccgtgtggtaagggaggggtttggctgctcgccaattgcaaggtgattcctg

SEQ ID NO: 43 - DNA, 59 bp, Homo sapiens
agctcttccgtgttgtaagggaggggtttggctgctcgccaattgcaaggtgattcctg

SEQ ID NO: 44 - DNA, 22 bp, Homo sapiens
tcttccgtgtgcttcccacact

SEQ ID NO: 45 - DNA, 22 bp, Homo sapiens
tcttccgtgttcttcccacact

SEQ ID NO: 46 - DNA, 57 bp, Homo sapiens
tcttccgtgttgtaagggaggggtttggctgctcgccaattgcaagcttcccacact

SEQ ID NO: 47 - RNA, 21 nt, Artificial Sequence
  AON10
cgagcagccaaaccccucccu

SEQ ID NO: 48 - RNA, 21 nt, Artificial Sequence
  AON11
ggcgagcagccaaaccccucc

SEQ ID NO: 49 - RNA, 21 nt, Artificial Sequence
  AON12
uuggcgagcagccaaaccccu

SEQ ID NO: 50 - RNA, 21 nt, Artificial Sequence
  AON13
aauuggcgagcagccaaaccc

SEQ ID NO: 51 - RNA, 21 nt, Artificial Sequence
  AON14
gcaauuggcgagcagccaaac

SEQ ID NO: 52 - RNA, 21 nt, Artificial Sequence
  AON15
uugcaauuggcgagcagccaa

SEQ ID NO: 53 - RNA, 21 nt, Artificial Sequence
  AON16
ccuugcaauuggcgagcagcc

SEQ ID NO: 54 - RNA, 21 nt, Artificial Sequence
  AON17
caccuugcaauuggcgagcag

SEQ ID NO: 55 - RNA, 21 nt, Artificial Sequence
  AON18
ucaccuugcaauuggcgagca

SEQ ID NO: 56 - RNA, 21 nt, Artificial Sequence
  AON19
aucaccuugcaauuggcgagc

SEQ ID NO: 57 - RNA, 21 nt, Artificial Sequence
  AON20
aaucaccuugcaauuggcgag

SEQ ID NO: 58 - RNA, 21 nt, Artificial Sequence
  AON21
gaaucaccuugcaauuggcga

SEQ ID NO: 59 - RNA, 21 nt, Artificial Sequence
  AON22
ggaaucaccuugcaauuggcg

SEQ ID NO: 60 - RNA, 21 nt, Artificial Sequence
  AON23
aggaaucaccuugcaauuggc

SEQ ID NO: 61 - RNA, 21 nt, Artificial Sequence
  AON24
caggaaucaccuugcaauugg

SEQ ID NO: 62 - RNA, 21 nt, Artificial Sequence
  AON25
ccaggaaucaccuugcaauug

SEQ ID NO: 63 - RNA, 21 nt, Artificial Sequence
  AON26
cccaggaaucaccuugcaauu

SEQ ID NO: 64 - RNA, 21 nt, Artificial Sequence
  AON27
ccccaggaaucaccuugcaau

SEQ ID NO: 65 - RNA, 21 nt, Artificial Sequence
  AON28
accccaggaaucaccuugcaa

SEQ ID NO: 66 - RNA, 21 nt, Artificial Sequence
  AON29
uaccccaggaaucaccuugca

SEQ ID NO: 67 - RNA, 21 nt, Artificial Sequence
  AON30
cuaccccaggaaucaccuugc

SEQ ID NO: 68 - RNA, 21 nt, Artificial Sequence
  AON31
gcuaccccaggaaucaccuug

SEQ ID NO: 69 - RNA, 21 nt, Artificial Sequence
  AON32
ugcuaccccaggaaucaccuu

SEQ ID NO: 70 - RNA, 21 nt, Artificial Sequence
  AON33
cugcuaccccaggaaucaccu

SEQ ID NO: 71 - RNA, 21 nt, Artificial Sequence
  AON34
ucugcuaccccaggaaucacc

SEQ ID NO: 72 - RNA, 21 nt, Artificial Sequence
  AON35
ggaaucaccuugcaauuggcg

SEQ ID NO: 73 - RNA, 21 nt, Artificial Sequence
  AON36
gcaauuggcgagcagccaaac

SEQ ID NO: 74 - RNA, 21 nt, Artificial Sequence
  AON37
uugcaauuggcgagcagccaa

SEQ ID NO: 75 - RNA, 21 nt, Artificial Sequence
  AON38
ccuugcaauuggcgagcagcc

SEQ ID NO: 76 - RNA, 21 nt, Artificial Sequence
  AON39
aucaccuugcaauuggcgagc

SEQ ID NO: 77 - RNA, 21 nt, Artificial Sequence
  AON40
aggaaucaccuugcaauuggc

SEQ ID NO: 78 - RNA, 21 nt, Artificial Sequence
  2′-OMe SON
cgccaauugcaaggugauucc

SEQ ID NO: 79 - RNA, 21 nt, Artificial Sequence
  2′-MOE SON
cgccaauugcaaggugauucc

SEQ ID NO: 80 - DNA, 75 bp, Artificial Sequence
  Mutant ABCA4 in Figure 4
cttccgtgttgtaagggaggggtttggctgctcgccaattgcaaggtgattcctggggtagcagagcctcacgaa

SEQ ID NO: 81 - DNA, 21 bp, Artificial Sequence
  ABCA4 ex6_Fw primer
cttcagccagagacgcggggc

SEQ ID NO: 82 - DNA, 20 bp, Artificial Sequence
  pCI-Neo-Rho ex5_Rev primer
aggtgtaggggatgggagac

SEQ ID NO: 83 - DNA, 18 bp, Artificial Sequence
  pCI-Neo-Rho ex5_Fw primer
atctgctgcggcaagaac

SEQ ID NO: 84 - DNA, 20 bp, Artificial Sequence
  pCI-Neo-Rho ex5_Rev
aggtgtaggggatgggagac

SEQ ID NO: 85 - DNA, 24 bp, Artificial Sequence
  exon5_Fw primer
ggaatacgaataagggatatcttg

SEQ ID NO: 86 - DNA, 24 bp, Artificial Sequence
  exon7_Rev primer
cttgaattcttggtgacatatcag

SEQ ID NO: 87 - DNA, 19 bp, Artificial Sequence
  exon4_Fw primer
cacccggagagaattgcag

SEQ ID NO: 88 - DNA, 19 bp, Artificial Sequence
  exon8_Rev primer
cctgcatactcggccgatg

SEQ ID NO: 89 - DNA, 24 bp, Artificial Sequence
  exon5_Fw primer
ggaatacgaataagggatatcttg

SEQ ID NO: 90 - DNA, 24 bp, Artificial Sequence
  exon7_Rev primer
cttgaattcttggtgacatatcag

SEQ ID NO: 91 - DNA, 20 bp, Artificial Sequence
  P49750_Fw_ACTB exon 3 primer
actgggacgacatggagaag

SEQ ID NO: 92 - DNA, 20 bp, Artificial Sequence
  P49751_Rev_ACTB exon 4 primer
tctcagctgtggtggtgaag

(19) United States
(12) Patent Application Publication
(10) Pub. No.: US 2022/0049250 A1
(43) Pub. Date: Feb. 17, 2022

(54) Antisense Oligonucleotides Rescue Aberrant Splicing of ABCA4

(71) Applicant: Stichting Katholieke Universiteit, Nijmegen (NL)

(72) Inventors: Robert Wilhelmus Johanna Collin, Venlo (NL); Alejandro Garanto Iglesias, Nijmegen (NL); Franciscus Peter Maria Cremers, Malden (NL)

(73) Assignee: Stichting Katholieke Universiteit, Nijmegen (NL)

(21) Appl. No.: 17/298,957

(22) PCT Filed: Dec. 4, 2019
(86) PCT No.: PCT/EP2019/083618

§ 371 Date: Jun. 2, 2021

(30) Foreign Application Priority Data
Dec. 4, 2018 (EP) 18210107.1

(51) Int. Cl. C12N 15/113

(52) U.S. Cl. CPC C12N 15/113

(57) ABSTRACT

The present invention relates to the field of medicine. In particular, it relates to novel antisense oligonucleotides that may be used in the treatment, prevention and/or delay of Stargardt disease

### FIELD OF THE INVENTION

The invention relates to the fields of medicine and immunology. In particular, it relates to novel antisense oligonucleotides that may be used in the treatment, prevention and/or delay of an ABCA4-associated condition.

### BACKGROUND OF THE INVENTION

Autosomal recessive mutations in ABCA4 cause Stargardt disease, a progressive disorder characterized by central vision loss and often leading to complete blindness. A typical hallmark of Stargardt disease is the presence of many yellow spots (flecks) distributed throughout the fundus of the patients. The ABCA4 gene is comprised of 50 exons and encodes a protein consisting of 2273 amino acids. This protein is expressed in the outer segments of cone and rod photoreceptor cells and plays an important role in the removal of waste products following phototransduction.

Besides STGD1, variants in ABCA4 can also lead to other subtypes of retinal disease ranging from bull's eye maculopathy to autosomal recessive cone-rod dystrophy (arCRD; Cremers et al, 1998; Maugeri et al, 2000) and pan-retinal dystrophies (Cremers et al, 1998; Martinez-Mir et al, 1998), depending on the severity of the alleles.

Biallelic ABCA4 variants can be identified in approximately 80% of the cases with STGD1 (Allikmets et al, 1997; Fujinami et al, 2013; Lewis et al, 1999; Maugeri et al, 1999; Rivera et al, 2000; Schulz et al, 2017; Webster et al, 2001; Zernant et al, 2011; Zernant et al, 2017), and 30% of cases with arCRD (Maugeri et al, 2000), after sequencing coding regions and flanking splice sites. In general, individuals with arCRD or pan-retinal dystrophy carry two severe ABCA4 alleles, whereas individuals with STGD1 carry two moderately severe variants or a combination of a mild and a severe variant (Maugeri et al, 1999; van Driel et al, 1998). It has been hypothesized that the majority of the missing ABCA4 variants in STGD1 patients reside in intronic regions of the gene, and indeed, over the last few years, several groups have demonstrated the existence of such deep-intronic variants (Bauwens et al, 2015; Bax et al, 2015; Braun et al, 2013; Lee et al, 2016; Schulz et al, 2017). A mutation that is frequently present in patients with STGDI (−2-3% of all STGDI cases in the Western world carry this mutation) is c.768G>T, a variant that affects the last nucleotide of exon 6. This mutation weakens the splice donor site of exon 6, resulting in the use of an alternative splice donor site downstream in intron 6 and a subsequent elongation of the transcript with 35 nucleotides. This elongation is predicted to result in a frame-shift and thus premature termination of ABCA4 protein synthesis.

The fact that a considerable amount of STGD1 cases carries the c.768G>T variant renders it an attractive target for antisense oligonucleotide (AON)-based splice modulation therapy. Accordingly, there is an urge to develop AONs for splice modulation of the ABCA4 gene to enable expression of a functional ABCA4 protein in subjects suffering from Stargardt disease, in particular for the c.768G>T mutation.

### SUMMARY OF THE INVENTION

In a first aspect, the invention provides for an antisense oligonucleotide for redirecting splicing that binds to and/or is complementary to a polynucleotide with the nucleotide sequence as shown in SEQ ID NO: 4, preferably the antisense oligonucleotide binds to or is complementary to a polynucleotide with SEQ ID NO: 5, more preferably the antisense oligonucleotide binds to or is complementary to a polynucleotide with SEQ ID NO: 80, even more preferably the antisense oligonucleotide binds to or is complementary to a polynucleotide selected from the group consisting of SEQ ID NO: 7, 8, 9; 11, 12,13; 15,16,17; 19, 20, and 21.

In a second aspect, the invention provides for a viral vector expressing antisense oligonucleotide for redirecting splicing as defined herein when placed under conditions conducive to expression of the molecule.

In a third aspect, the invention provides for a pharmaceutical composition comprising an antisense oligonucleotide for redirecting splicing as defined herein or a viral vector as defined herein and a pharmaceutically acceptable excipient.

In a fourth aspect, the invention provides for an antisense oligonucleotide for redirecting splicing as defined herein for use as a medicament, preferably for use as a medicament for treating an ABCA4 related disease or a condition requiring modulating splicing of ABCA4.

### DETAILED DESCRIPTION OF THE INVENTION

By definition, antisense oligonucleotides (AONs) are substantially complementary (antisense) to their target, allowing them to bind to the corresponding pre-mRNA molecule, thereby, without wishing to be being bound by theory, preventing the binding of proteins essential for splicing. Usually, this lack of binding results in the skipping of the targeted exon, as the present inventors have previously shown for several mutations in ABCA4 (WO2018/109011).

Some mutations create novel splice acceptor, splice donor or exonic splice enhancer binding sites which results in the inclusion of pseudoexons to the mRNA of the corresponding gene. The c.768G>T mutation weakens the splice donor site of exon 6, resulting in the use of an alternative splice donor site downstream in intron 6 and a subsequent elongation of the transcript with 35 nucleotides. To restore splicing in individuals carrying the c.768G>T mutation, the inventors have designed AONs that specifically block the newly used alternative splice donor site in intron 6, and thereby redirect the splicing machinery back to the original site at position c.768.

Accordingly, in a first aspect the invention provides an antisense oligonucleotide for redirecting splicing that binds to and/or is complementary to a polynucleotide with the nucleotide sequence as shown in SEQ ID NO: 4. Preferably the antisense oligonucleotide binds to or is complementary to a polynucleotide with SEQ ID NO: 5. More preferably the antisense oligonucleotide binds to or is complementary to a polynucleotide with SEQ ID NO: 80. Even more preferably the antisense oligonucleotide binds to or is complementary to a polynucleotide selected from the group consisting of SEQ ID NO: 7, 8, 9; 11, 12,13; 15,16,17; 19, 20, and 21.

The terms “antisense oligonucleotide” or “AON” are used interchangeably herein and are understood to refer to an oligonucleotide molecule comprising a nucleotide sequence which is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions. Binding of an AON to its target can easily be assessed by the person skilled in the art using techniques that are known in the field such as the gel mobility shift assay as described in EP1619249.

The term “complementary” used in the context of the invention indicates that some mismatches in the antisense sequence are allowed as long as the functionality, i.e. redirecting splicing is achieved. Preferably, the complementarity is from 90% to 100%. In general this allows for 1 or 2 mismatches in an AON of 20 nucleotides or 1, 2, 3 or 4 mismatches in an AON of 40 nucleotides, or 1, 2, 3, 4, 5 or 6 mismatches in an AON of 60 nucleotides, etc. Optionally, said AON may further be tested by transfection into retina cells of patients. The complementary regions are preferably designed such that, when combined, they are specific for the exon in the pre-mRNA. Such specificity may be created with various lengths of complementary regions, as this depends on the actual sequences in other (pre-)mRNA molecules in the system. The risk that the AON will also be able to hybridize to one or more other pre-mRNA molecules decreases with increasing size of the AON. It is clear that AONs comprising mismatches in the region of complementarity but that retain the capacity to hybridize and/or bind to the targeted region(s) in the pre-mRNA, can be used in the invention. However, preferably at least the complementary parts do not comprise such mismatches as AONs lacking mismatches in the complementary part typically have a higher efficiency and a higher specificity than AONs having such mismatches in one or more complementary regions. It is thought, that higher hybridization strengths, (i.e. increasing number of interactions with the opposing strand) are favorable in increasing the efficiency of the process of interfering with the splicing machinery of the system.

The terms “modulate splicing” and “redirect splicing” are used herein interchangeably and encompass AON-based splice modulation therapy for the c.768G>T mutation. The term “redirecting splicing” is herein defined as redirecting the ABCA4 pre-mRNA splicing to yield the original transcript.

The AON according to the invention preferably does not contain a stretch of CpG, more preferably does not contain any CpG. The presence of a CpG or a stretch of CpG in an oligonucleotide is usually associated with an increased immunogenicity of said oligonucleotide (Dorn and Kippenberger, 2008). This increased immunogenicity is undesired since it may induce damage of the tissue to be treated, i.e. the eye. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an AON according to the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay. The AON according to the invention furthermore preferably has acceptable RNA binding kinetics and/or thermodynamic properties. The RNA binding kinetics and/or thermodynamic properties are at least in part determined by the melting temperature of an oligonucleotide (Tm; calculated with the oligonucleotide properties calculator (www.unc.edu/-cail/biotool/oligo/index) for single stranded RNA using the basic Tm and the nearest neighbor model), and/or the free energy of the AON-target exon complex (using RNA structure version 4.5). If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

In all embodiments, the nucleotide in the antisense oligonucleotide according to the invention may be an RNA residue, a DNA residue, or a nucleotide analogue or equivalent.

A preferred AON for redirecting splicing according to the invention, has a length of from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides. Preferably, an AON according to the invention has a length of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 , 38, 39, or 40 nucleotides.

In a preferred embodiment, an AON for redirecting splicing according to the invention, comprises or consists of SEQ ID NO: 34, 51, 52, 53, 54, 55, 56, 57, 58, 59, 60, 61, 73, 75 or 77. It was found that these AONs ware very efficient in redirecting the aberrant splicing of ABCA4 that is caused by the c.768G>T mutation. These preferred AONs preferably comprises from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides, or preferably comprises or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 , 38, 39, or 40 nucleotides.

It is preferred that an AON for redirecting splicing according to the invention comprises one or more residues that are modified to increase nuclease resistance, and/or to increase the affinity of the antisense oligonucleotide for the target sequence. Therefore, in a preferred embodiment, the AON comprises at least one nucleotide analogue or equivalent, wherein a nucleotide analogue or equivalent is defined as a residue having a modified base, and/or a modified backbone, and/or a non-natural internucleoside linkage, or a combination of these modifications.

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report, demonstrated triplex formation by a morpholino oligonucleotide and, because of the non-ionic backbone, these studies showed that the morpholino oligonucleotide was capable of triplex formation in the absence of magnesium.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005). Since the backbone of a PNA molecule contains no charged phosphate groups, PNA-RNA hybrids are usually more stable than RNA-RNA or RNA-DNA hybrids, respectively (Egholm et al., 1993). A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, a nucleotide analogue or equivalent according to the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and am inoalkylphosphoram idate, thionophosphoramidate, thionoalkylphosphonate, thionoalkylphosphotriester, selenophosphate or boranophosphate.

A further preferred nucleotide analogue or equivalent according to the invention comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-or N-alkynyl; O-, S-, or N-allyl; O-alkyl-O-alkyl, -methoxy, -aminopropoxy; methoxyethoxy; dimethylaminooxyethoxy; and -dimethylaminoethoxyethoxy. The sugar moiety can be a pyranose or derivative thereof, or a deoxypyranose or derivative thereof, preferably ribose or derivative thereof, or deoxyribose or derivative of. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O, 4′-C-ethylene-bridged nucleic acid (Morita et al., 2001). These substitutions render the nucleotide analogue or equivalent RNase H and nuclease resistant and increase the affinity for the target RNA.

In another embodiment, a nucleotide analogue or equivalent according to the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by the skilled person that it is not necessary for all positions in an AON to be modified uniformly. In addition, more than one of the aforementioned analogues or equivalents may be incorporated in a single AON or even at a single position within an AON. In certain embodiments, an AON according to the invention has at least two different types of analogues or equivalents.

Accordingly, in a preferred embodiment an antisense oligonucleotide for redirecting splicing according to the invention, comprises a 2′-O alkyl phosphorothioate antisense oligonucleotide, such as 2′-O-methyl modified ribose (RNA), 2′O-Ethyl modified ribose, 2′O-methoxyethyl modified ribose, 2′-O-propyl modified ribose, and/or substituted derivatives of these modifications such as halogenated derivatives.

In a preferred embodiment, an AON for redirecting splicing according to the invention, comprises or consists of SEQ ID NO: 34, 51, 52, 53, 54, 55, 56, 57, 58, 59, 60, 61, 73, 75 or 77, and comprises a 2′-O-methyl modified ribose (RNA) and a phosphorothioate backbone. In another preferred embodiment, an AON for redirecting splicing according to the invention, comprises or consists of SEQ ID NO: 34, 51, 52, 53, 54, 55, 56, 57, 58, 59, 60, 61, 73, 75 or 77, and comprises a 2′-O-methoxyethyl modified ribose (RNA) and a phosphorothioate backbone.

In an embodiment, an AON for redirecting splicing according to the invention, comprises or consists of SEQ ID NO: 6, and comprises a 2′-O-methyl modified ribose (RNA) or a 2′-O-methoxyethyl modified ribose (RNA) and a phosphorothioate backbone.

In an embodiment, an AON for redirecting splicing according to the invention, comprises or consists of SEQ ID NO: 10, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 14, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 18, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 22, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 26, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 30, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 47, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 48, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 49, and comprises a 2′-O-methyl modified ribose (RNA) or a 2′O-methoxyethyl modified ribose (RNA)and a phosphorothioate backbone.

In an embodiment, an AON for redirecting splicing according to the invention, comprises or consists of SEQ ID NO: 50, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 51, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 52, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 53, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 54, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 55, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 56, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 57, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 58, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 59, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 60, and comprises a 2′-O-methyl modified ribose (RNA) or a 2′O-methoxyethyl modified ribose (RNA)and a phosphorothioate backbone.

In an embodiment, an AON for redirecting splicing according to the invention, comprises or consists of SEQ ID NO: 61, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 62, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 63, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 64, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 65, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 66, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 67, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 68, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 69, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 70, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 71, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 72, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 73, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 74, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 75, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 76, and comprises a 2′-O-methyl modified ribose (RNA) or a 2′-O-methoxyethyl modified ribose (RNA)and a phosphorothioate backbone.

In an embodiment, an AON for redirecting splicing according to the invention, comprises or consists of SEQ ID NO: 77, and comprises a 2′-O-methyl modified ribose (RNA) or a 2′-O-methoxyethyl modified ribose (RNA)and a phosphorothioate backbone.

An AON for redirecting splicing according to the invention may be indirectly administrated using suitable means known in the art. It may for example be provided to an individual or a cell, tissue or organ of said individual as such, as a so-called ‘naked’ AON. It may also be administered in the form of an expression vector wherein the expression vector encodes an RNA transcript comprising the sequence of said AON according to the invention. The expression vector is preferably introduced into a cell, tissue, organ or individual via a gene delivery vehicle. In a preferred embodiment, there is provided a viral-based expression vector comprising an expression cassette or a transcription cassette that drives expression or transcription of an AON for redirecting splicing according to the invention. Accordingly, the invention provides for a viral vector expressing antisense oligonucleotide for redirecting splicing according to the invention when placed under conditions conducive to expression of the molecule.

A cell can be provided with an AON for redirecting splicing according to the invention by plasmid-derived antisense oligonucleotide expression or viral expression provided by adenovirus- or adeno-associated virus-based vectors. Expression may be driven by an RNA polymerase II promoter (Pol II) such as a U7 RNA promoter or an RNA polymerase III (Pol III) promoter, such as a U6 RNA promoter. A preferred delivery vehicle is a viral vector such as an adeno-associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an AON for redirecting splicing according to the invention. Preferred for the invention are those vectors wherein transcription is driven from PolIII promoters, and/or wherein transcripts are in the form fusions with U1 or U7 transcripts, which yield good results for delivering small transcripts. It is within the skill of the artisan to design suitable transcripts. Preferred are PolIII driven transcripts, preferably, in the form of a fusion transcript with an U1 or U7 transcript. Such fusions may be generated as previously described (Gorman et al., 1998).

A preferred expression system for an AON for redirecting splicing according to the invention is an adenovirus associated virus (AAV)-based vector. Single chain and double chain AAV-based vectors have been developed that can be used for prolonged expression of antisense nucleotide sequences for highly efficient redirection of splicing. A preferred AAV-based vector, for instance, comprises an expression cassette that is driven by an RNA polymerase III-promoter (Pol III) or an RNA polymerase II promoter (Pol II). A preferred RNA promoter is, for example, a Pol III U6 RNA promoter, or a Pol II U7 RNA promoter.

The invention accordingly provides for a viral-based vector, comprising a Pol II or a Pol III promoter driven expression cassette for expression of an AON for redirecting splicing according to the invention.

An AAV vector according to the invention is a recombinant AAV vector and refers to an AAV vector comprising part of an AAV genome comprising an encoded AON for redirecting splicing according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAVS, AAV8, AAV9 and others. A protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector.

More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector.

More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector.

More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector.

A nucleic acid molecule encoding an AON for redirecting splicing according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art, see e.g. (Chiorini et al., 1999) or U.S. Pat. No. 5,139,941, incorporated herein by reference. The AAV helper functions can be supplied on an AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs according to the invention may thus be chosen such that they produce the desired combination of serotypes for the AAV vector's capsid protein shell on the one hand and for the AAV genome present in said AAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

Preferably, an AAV vector according to the invention is constructed and produced according to the method according to Garanto et al., 2016 which is herein incorporated by reference.

A preferred AAV vector according to the invention is an AAV vector, preferably an AAV2/5, AAV2/8, AAV2/9 or AAV2/2 vector, expressing an AON for redirecting splicing according to the invention that is an AON that comprises, or preferably consists of, a sequence that is:

complementary or substantially complementary to a nucleotide sequence consisting of SEQ ID NO: 4, preferably the AON is complementary to a polynucleotide with SEQ ID NO: 5, more preferably the AON is complementary to a polynucleotide with SEQ ID NO: 80, even more preferably complementary or substantially complementary to a polynucleotide with a nucleotide sequence selected from the group consisting of SEQ ID NO: 7, 8, 9; 11, 12,13; 15,16,17; 19, 20 and 21. Even more preferably, the AON comprises or consists of polynucleotide with a nucleotide sequence selected from the group consisting of SEQ ID NO: 6, SEQ ID NO: 10, SEQ ID NO: 14, SEQ ID NO: 18, SEQ ID NO: 22, SEQ ID NO: 26, and SEQ ID NO: 30, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ

ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or most preferably selected from the group consisting of SEQ ID NO: 34, 51, 52, 53, 54, 55, 56, 57, 58, 59, 60, 61, 73, 75 and 77.

Improvements in means for providing an individual or a cell, tissue, organ of said individual with an AON for redirecting splicing according to the invention, are anticipated considering the progress that has already thus far been achieved. Such future improvements may of course be incorporated to achieve the mentioned effect on restructuring of mRNA using a method according to the invention. An AON for redirecting splicing according to the invention can be delivered as such as a ‘naked’ AON to an individual, a cell, tissue or organ of said individual. When administering an AON for redirecting splicing according to the invention, it is preferred that the molecule is dissolved in a solution that is compatible with the delivery method. Retina cells can be provided with a plasmid for antisense oligonucleotide expression by providing the plasmid in an aqueous solution.

Alternatively, a preferred delivery method for an AON for redirecting splicing or a plasmid for expression of such AON is a viral vector or are nanoparticles. Preferably, viral vectors or nanoparticles are delivered to retina or other relevant cells. Such delivery to retina cells or other relevant cells may be in vivo, in vitro or ex vivo; see e.g. Garanto et al, 2016, which is herein incorporated by reference.

Alternatively, a plasmid can be provided by transfection using known transfection agents. For intravenous, subcutaneous, intramuscular, intrathecal and/or intraventricular administration it is preferred that the solution is a physiological salt solution. Particularly preferred in the invention is the use of an excipient or transfection agents that will aid in delivery of each of the constituents as defined herein to a cell and/or into a cell, preferably a retina cell. Preferred are excipients or transfection agents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection agentia comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), lipofectin™, DOTAP and/or viral capsid proteins that are capable of self-assembly into particles that can deliver each constitutent as defined herein to a cell, preferably a retina cell. Such excipients have been shown to efficiently deliver an oligonucleotide such as AONs to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles.

Polycations such as diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an AON according to the invention, across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an exon retention molecule for use in the current invention to deliver it for the prevention, treatment or delay of ABCA4-related disease or condition. “Prevention, treatment or delay of an ABCA4-related disease or condition” is herein preferably defined as preventing, halting, ceasing the progression of, or reversing partial or complete visual impairment or blindness that is caused by a genetic defect in the ABCA4 gene.

In addition, an AON for redirecting splicing according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, an AON for redirecting splicing according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may not be suitably formulated in one single combination or composition or preparation. Depending on their identity and specific features, the skilled person will know which type of formulation is the most appropriate for each constituent as defined herein. In a preferred embodiment, the invention provides a composition or a preparation which is in the form of a kit of parts comprising an AON for redirecting splicing according to the invention and a further adjunct compound as later defined herein.

If required and/or if desired, an AON for redirecting splicing according to the invention or a vector, preferably a viral vector, according to the invention, expressing an AON for redirecting splicing according to the invention can be incorporated into a pharmaceutically active mixture by adding a pharmaceutically acceptable carrier.

Accordingly, the invention also provides for a composition, preferably a pharmaceutical composition comprising an antisense oligonucleotide for redirecting splicing according to the invention or a viral vector according to the invention and a pharmaceutically acceptable excipient. Such composition may comprise a single AON for redirecting splicing or viral vector according to the invention, but may also comprise multiple, distinct AONs for redirecting splicing or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington, 2000. Each feature of said composition has earlier been defined herein.

A preferred route of administration is through intra-vitreal injection of an aqueous solution or specially adapted formulation for intraocular administration. EP2425 814 discloses an oil in water emulsion especially adapted for intraocular (intravitreal) administration of peptide or nucleic acid drugs. This emulsion is less dense than the vitreous fluid, so that the emulsion floats on top of the vitreous, avoiding that the injected drug impairs vision. Therefor in one embodiment, there is provided for a pharmaceutical composition suitable for intravitreal administration and dosed in an amount ranged from 0.01 and 20 mg/kg, preferably from 0.05 and 20 mg/kg of total antisense oligonucleotides per eye. A suitable intravitreal dose is provided and comprises between 0.05 mg and 5 mg, preferably between 0.1 and 1 mg of total antisense oligonucleotides per eye, such as about per eye: 0.1, 0.2, 0.3, 0.4, 0.5, 0.6, 0.7, 0.8, 0.9 or 1.0 mg.

A preferred AON for redirecting splicing according to the invention, is for the treatment of an ABCA4-related disease or condition of an individual. In all embodiments of the invention, the term “treatment” is understood to include the prevention and/or delay of the ABCA4-related disease or condition. An individual, which may be treated using an AON for redirecting splicing according to the invention may already have been diagnosed as having an ABCA4-related disease or condition. Alternatively, an individual which may be treated using an AON for redirecting splicing according to the invention may not have yet been diagnosed as having a ABCA4-related disease or condition but may be an individual having an increased risk of developing a ABCA4-related disease or condition in the future given his or her genetic background. A preferred individual is a human being. In all embodiments of the invention, the ABCA4-related disease or condition preferably is Stargardt disease.

Accordingly, the invention further provides for an antisense oligonucleotide for redirecting splicing according to the invention, or a viral vector according to the invention, or a (pharmaceutical) composition according to the invention for use as a medicament, preferably as a medicament for the treatment of an ABCA4-related disease or condition requiring modulating splicing of ABCA4 and for use as a medicament for the prevention, treatment or delay of an ABCA4-related disease or condition. Each feature of all medical use embodiment herein has earlier been defined herein and is preferably such feature as earlier defined herein.

The invention further provides for the use of an AON for redirecting splicing according to the invention, a vector according to the invention or a (pharmaceutical) composition according to the invention for treating an ABCA4-related disease or condition requiring modulating splicing of ABCA4. Each feature of all medical use embodiment herein has earlier been defined herein and is preferably such feature as earlier defined herein.

The invention further provides for, a method of treatment of an ABCA4-related disease or condition requiring modulating splicing of ABCA4, comprising said method comprising contacting a cell of said individual with an AON for redirecting splicing according to the invention, a vector according to the invention or a (pharmaceutical) composition according to the invention. Each feature of all medical use embodiment herein has earlier been defined herein and is preferably such feature as earlier defined herein.

The invention further provides for the use of an AON for redirecting splicing according to the invention, a vector according to the invention or a (pharmaceutical) composition according to the invention for the preparation of a medicament for the treatment of an ABCA4-related disease or condition requiring modulating splicing of ABCA4. Each feature of all medical use embodiment herein has earlier been defined herein and is preferably such feature as earlier defined herein.

The invention further provides for an antisense oligonucleotide for redirecting splicing according to the invention, the use according the invention or the method according to the invention, wherein the ABCA4-related disease or condition is Stargardt disease.

Treatment in a use or in a method according to the invention is preferably at least once, and preferably lasts at least one week, one month, several months, one year, 2, 3, 4, 5, 6 years or longer, such as life-long. Each AON for redirecting splicing according to the invention or equivalent thereof as defined herein for use according to the invention may be suitable for direct administration to a cell, tissue and/or an organ in vivo of individuals already affected or at risk of developing an ABCA4-related disease or condition, and may be administered directly in vivo, ex vivo or in vitro. The frequency of administration of an AON, composition, compound or adjunct compound according to the invention may depend on several parameters such as the severity of the disease, the age of the patient, the mutation of the patient, the number of AON for redirecting splicing according to the invention (i.e. dose), the formulation of the AON, composition, compound or adjunct compound according to the invention, the route of administration and so forth. The frequency of administration may vary between daily, weekly, at least once in two weeks, or three weeks or four weeks or five weeks or a longer time period.

Dose ranges of an AON, composition, compound or adjunct compound according to the invention are preferably designed on the basis of rising dose studies in clinical trials (in vivo use) for which rigorous protocol requirements exist. An AON according to the invention may be used at a dose which is ranged from 0.01 and 20 mg/kg, preferably from 0.05 and 20 mg/kg. A suitable intravitreal dose would be between 0.05 mg and 5 mg, preferably between 0.1 and 1 mg per eye, such as about per eye: 0.1, 0.2, 0.3, 0.4, 0.5, 0.6, 0.7, 0.8, 0.9 or 1.0 mg.

In a preferred embodiment, a concentration of an oligonucleotide as defined herein, which is ranged from 0.1 nM and 1 μM is used. Preferably, this range is for in vitro use in a cellular model such as retina cells or retinal tissue. More preferably, the concentration used is ranged from 1 to 400 nM, even more preferably from 10 to 200 nM, even more preferably from 50 to 100 nM. If multiple distinct AONs are used, this concentration or dose may refer to the total concentration or dose of the AONs or the concentration or the dose of each AON added.

In a preferred embodiment, a viral vector, preferably an AAV vector as described earlier herein, as delivery vehicle for a molecule according to the invention, is administered in a dose ranging from 1×1⁰⁹−1×10¹⁷ virus particles per injection, more preferably from 1×10¹⁰−1 ×10¹² virus particles per injection.

The ranges of concentration or dose of AONs as depicted above are preferred concentrations or doses for in vivo, in vitro or ex vivo uses. The skilled person will understand that depending on the AONs used, the target cell to be treated, the gene target and its expression levels, the medium used and the transfection and incubation conditions, the concentration or dose of AONs used may further vary and may need to be optimized any further.

An AON for redirecting splicing according to the invention, or a viral vector according to the invention, or a composition according to the invention for use according to the invention may be administered to a cell, tissue and/or an organ in vivo of individuals already affected or at risk of developing a ABCA4-related disease or condition, and may be administered in vivo, ex vivo or in vitro. An AON for redirecting splicing according to the invention, or a viral vector according to the invention, or a composition according to the invention may be directly or indirectly administered to a cell, tissue and/or an organ in vivo of an individual already affected by or at risk of developing a ABCA4-related disease or condition, and may be administered directly or indirectly in vivo, ex vivo or in vitro. As Stargardt disease has a pronounced phenotype in retina cells, it is preferred that said targeted cells are retina cells, it is further preferred that said tissue is the retina and it is further preferred that said organ comprises or consists of the eye.

The invention further provides for a method for a method for modulating splicing of ABCA4 in a cell, said method comprising contacting the cell, preferably a retina cell, with an antisense oligonucleotide for redirecting splicing according to the invention, the vector according to the invention or the pharmaceutical composition according to the invention The features of this aspect are preferably those defined earlier herein. Contacting the cell with an AON for redirecting splicing according to the invention, or a viral vector according to the invention, or a composition according to the invention may be performed by any method known by the person skilled in the art. Use of the methods for delivery of AONs for redirecting splicing, viral vectors and compositions as described earlier herein is included. Contacting may be directly or indirectly and may be in vivo, ex vivo or in vitro.

Unless otherwise indicated each embodiment as described herein may be combined with another embodiment as described herein.

Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value. The sequence information as provided herein should not be so narrowly construed as to require inclusion of erroneously identified bases. The skilled person is capable of identifying such erroneously identified bases and knows how to correct for such errors. In case of sequence errors, the sequence of the polypeptide obtainable by expression of the gene present in SEQ ID NO: 1 containing the nucleic acid sequence coding for the polypeptide should prevail.

All patent and literature references cited in the present specification are hereby incorporated by reference in their entirety.

### DESCRIPTION OF THE FIGURES

FIG. 1: A) Bioinformatic prediction of splice donor site strength for the original exon 6 splice donor site, and the alternative splice donor site in intron 6, both for the wild-type (upper sequence) and c.768G>T mutant (lower sequence) situation. The usage of the alternative splice site in the mutant situation is predicted to result in a 35-nt extension (in gray, red in the colored version) of the ABCA4 transcript. B) RT-PCR analysis of ABCA4 on RNA derived from fibroblast of a control (ctrl). individual, a STGD1 patient compound heterozygous (comp. het.) for the c.768G>T change, and a STGD1 patient homozygous (hom.) for the c.768G>T mutation. − or + indicates the absence or presence of cycloheximide. M: 100-bp ladder. MQ: milliQ water.

FIG. 2: Schematic representation of the location of the c.768G>T variant, the original and alternative splice donor site, and the relative position of the AONs that were designed.

FIG. 3: RNA analysis of homozygous c.768G>T patient-derived fibroblast cells upon AON delivery.

(A) Analysis of ABCA4 transcript by RT-PCR and nested PCR of patient-derived fibroblast cells treated with AON2 to AON8 or non-treated (NT). Wild-type fibroblast cells (WT) were used as control. ACTB was used as loading control. MQ stands for the negative control of the first and second (nested) PCR reaction. AON1 gave similar results to AON2 (data not shown).

(B) Chromatograms of the bands highlighted in the gel 1 to 3. The band with the asterisk turned out to be contamination as the sequence only showed a peak representing a G, like in band 1, and the patient homozygously carries the c.768G>T mutation. AON8 was able to correct splicing as shown by the sequence in band 2.

FIG. 4: AON positions compared to the target region

Position of the AONs relative to the target region. AONs10-34 contained the 2′-OMe modification with a phosphorothioate (PS) backbone, whereas AONs35-40 contained the 2′-O-MOE modification with a phosphorothioate (PS) backbone. The exonic part is depicted with a grey box. The c.768G>T mutation is indicated with an arrow and in bold. The 35-nucleotide extension found in the STGD1 cases is listed in italics.

FIG. 5: AON screening in HEK293T cells using a midigene harboring the c.768T variant

RT-PCR analysis of HEK293T cells co-transfected with the midigene carrying the c.768T variant (MUT) and either an AON (A#) or the sense oligo (SON) with the 2′-OMe modification with a phosphorothioate (PS) backbone. A wild-type midigene carrying the c.768G (WT) was used as a control. Non-transfected HEK293T cells (HEK) were used as negative control. MQ indicates the negative control of the PCR. In the lower panel, semi-quantification of the ratio (expressed in percentage) between correct (WT) and aberrant (MUT) transcript for each condition is presented.

FIG. 6: Screening of 2′-O-MOE AONs in HEK293T cells

RT-PCR analysis of HEK293T cells co-transfected with the mutant midigene (MUT) and several AONs (A#) or sense oligo (SON) with the 2′-MOE chemical modification and a PS backbone. A wild-type midigene (WT) was used as a positive control, while non-transfected cells (HEK) were used as negative control. MQ lane corresponds to the negative control of the PCR. The lower panel represents the semi-quantification of the bands expressed in percentage of correct (WT) and aberrant (MUT) transcript. AON concentration: 1 μM.

FIG. 7: AON efficacy analysis in patient-derived fibroblast cells

Analysis of the efficacy of several AONs (A#) either 2′-OMe or 2′-MOE chemically modified with a PS backbone in a patient-derived fibroblast cell line homozygously carrying the c.768G>T variant (HOM). Fibroblast derived from a healthy individual were used as control (CON). Since the aberrant transcript is subjected to nonsense-mediated decay (NMD) both CON and HOM cells were cultured in the absence (−) or presence (+) of cycloheximide (CHX). All conditions involving AON delivery are performed under the presence of CHX. MQ indicates the negative control of the PCR. ACTB was used as loading control. Semi-quantitative analysis of the ratio of correct (WT) and aberrant (MUT) transcripts expressed in percentage for each condition. AON concentration: 1 μM.

### DESCRIPTION OF THE SEQUENCES

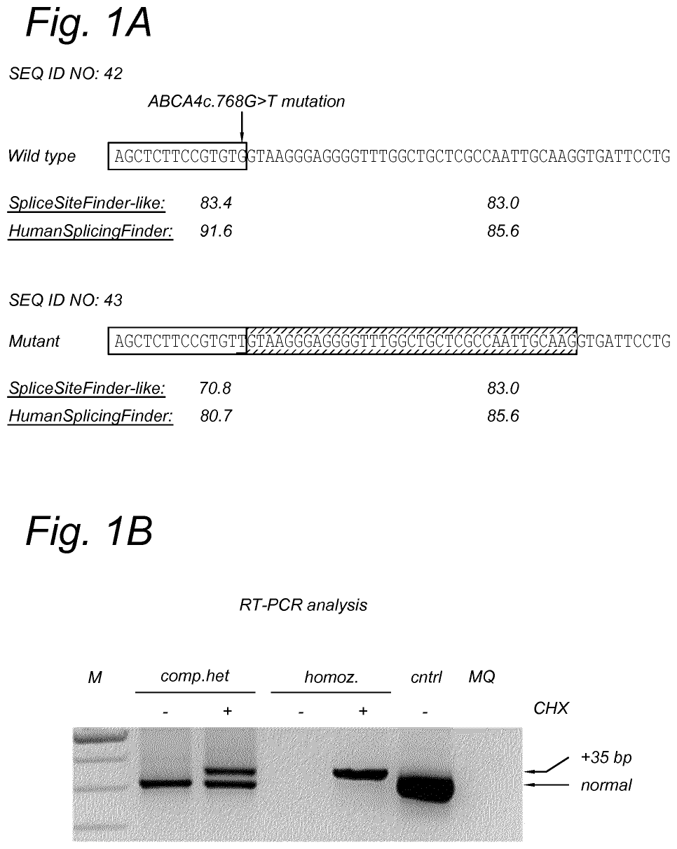

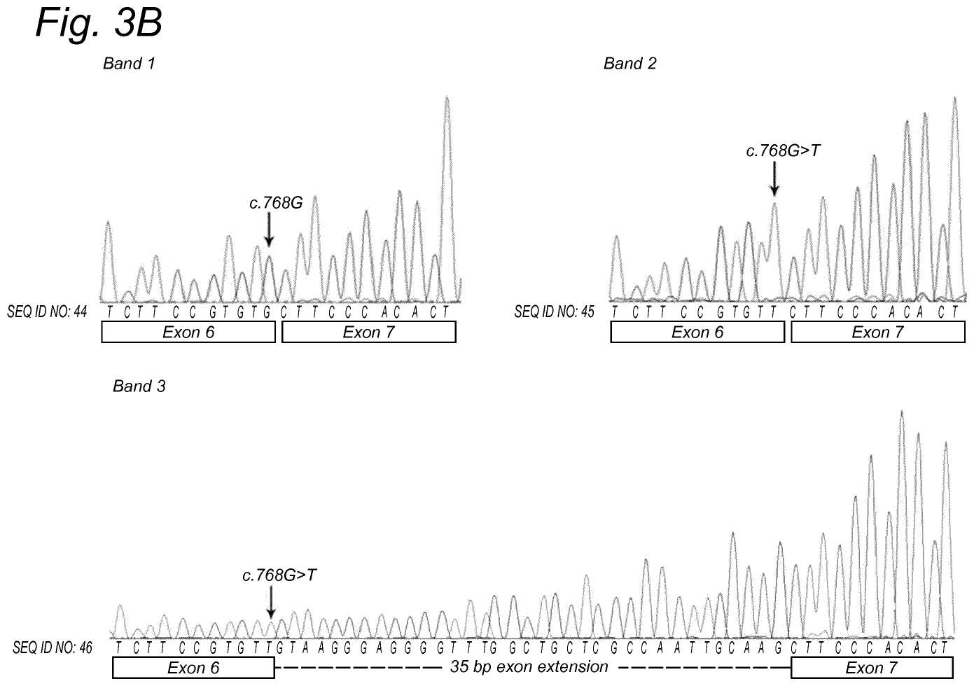

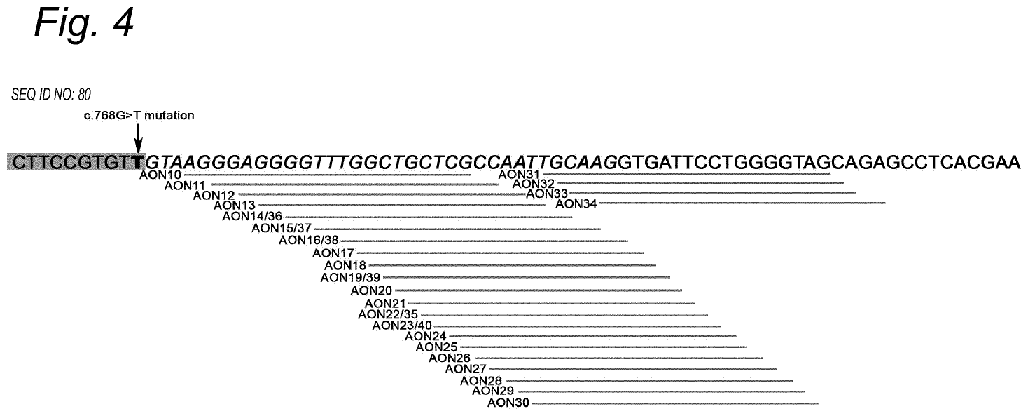

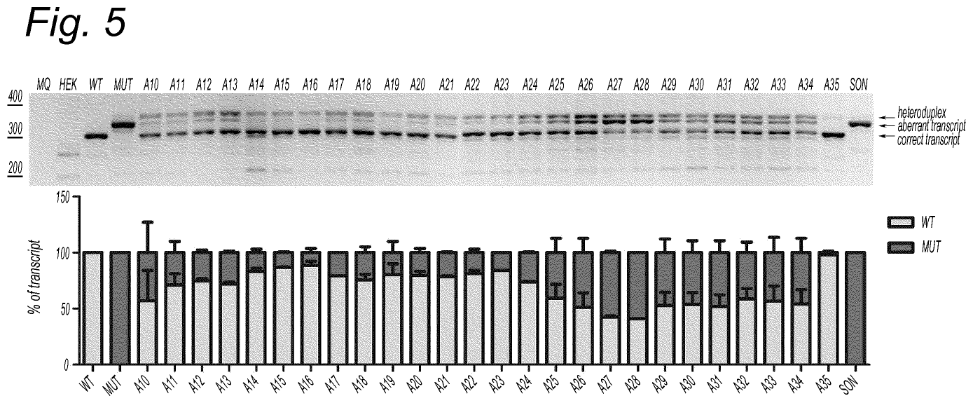

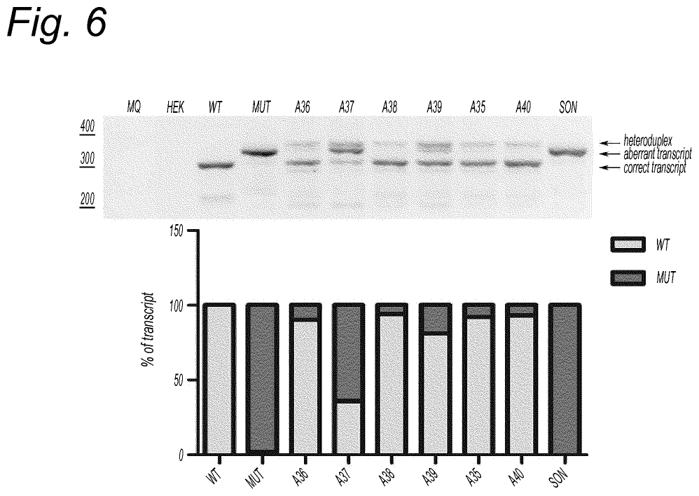

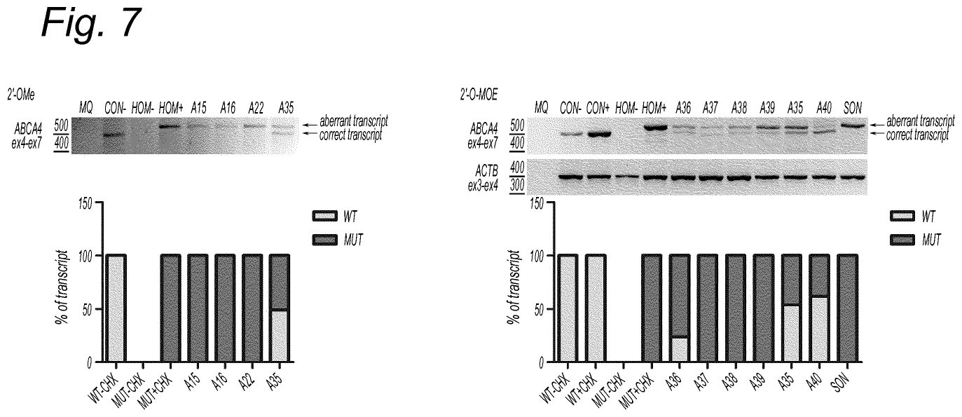

TABLE 1
Sequences

| SEQ ID NO: | Name |
|---|---|
| 1 | Genomic DNA sequence ABCA4 |
| 2 | mRNA sequence ABCA4 |
| 3 | ABCA4 Protein sequence |
| 4 | Exon 6 sequence ((±75 bp) |
| 5 | Target region in exon 6 |
| 6 | AON1 |
| 7 | AON1 target region |
| 8 | AON1 target region +5 |
| 9 | AON1 target region +10 |
| 10 | AON2 |
| 11 | AON2 target region |
| 12 | AON2 target region +5 |
| 13 | AON2 target region +10 |
| 14 | AON3 |
| 15 | AON3 target region |
| 16 | AON3 target region +5 |
| 17 | AON3 target region +10 |
| 18 | AON4 |
| 19 | AON4 target region |
| 20 | AON4 target region +5 |
| 21 | AON4 target region +10 |
| 22 | AON5 |
| 23 | AON5 target region |
| 24 | AON5 target region +5 |
| 25 | AON5 target region +10 |
| 26 | AON6 |
| 27 | AON6 target region |
| 28 | AON6 target region +5 |
| 29 | AON6 target region +10 |
| 30 | AON7 |
| 31 | AON7 target region |
| 32 | AON7 target region +5 |
| 33 | AON7 target region +10 |
| 34 | AON8 |
| 35 | AON8 target region |
| 36 | AON8 target region +5 |
| 37 | AON8 target region +10 |
| 38 | exon4_Fw primer |
| 39 | Exon8_Rv primer |
| 40 | exon5_Fw primer |
| 41 | exon7_Rv primer |
| 42 | Wild-type ABCA4 sequence in FIG. 1A |
| 43 | Mutant ABCA4 sequence in FIG. 1A |
| 44 | Sequence of the fragment in band 1 in FIG. 3B |
| 45 | Sequence of the fragment in band 2 in FIG. 3B |
| 46 | Sequence of the fragment in band 3 in FIG. 3B |
| 47 | AON10 |
| 48 | AON11 |
| 49 | AON12 |
| 50 | AON13 |
| 51 | AON14 |
| 52 | AON15 |
| 53 | AON16 |
| 54 | AON17 |
| 55 | AON18 |
| 56 | AON19 |
| 57 | AON20 |
| 58 | AON21 |
| 59 | AON22 |
| 60 | AON23 |
| 61 | AON24 |
| 62 | AON25 |
| 63 | AON26 |
| 64 | AON27 |
| 65 | AON28 |
| 66 | AON29 |
| 67 | AON30 |
| 68 | AON31 |
| 69 | AON32 |
| 70 | AON33 |
| 71 | AON34 |
| 72 | AON35 |
| 73 | AON36 |
| 74 | AON37 |
| 75 | AON38 |
| 76 | AON39 |
| 77 | AON40 |
| 78 | 2′-OMe SON |
| 79 | 2′-MOE SON |
| 80 | Mutant ABCA4 sequence in FIG. 4 |
| 81 | ABCA4 ex6_Fw primer |
| 82 | pCI-Neo-Rho ex5_Rev primer |
| 83 | pCI-Neo-Rho ex5_Fw primer |
| 84 | pCI-Neo-Rho ex5_Rev primer |
| 85 | exon5_Fw primer |
| 86 | exon7_Rev primer |
| 87 | exon4_Fw primer |
| 88 | exon8_Rev primer |
| 89 | exon5_Fw primer |
| 90 | exon7_Re primer |
| 91 | P49750_Fw_ACTB exon 3 primer |
| 92 | P49751_Rev_ACTB exon 4 |

### Examples

A recurrent mutation in ABCA4 that underlies Stargardt disease (STGD1) is c.768G>T. This mutation changes the last nucleotide of exon 6, but does not alter the amino acid sequence, i.e. the Valine residue encoded by the corresponding codon remains a Valine (p.Va1256Val). Instead, the strength of the splice donor site of exon 6 decreases by the presence of a T instead of a G (FIG. 1A, FIG. 4). As a result, an alternative splice site 35-nt downstream in intron 6 is being used by the splicing machinery, leading to a 35nt-extension of exon 6, as demonstrated in patient-derived fibroblast cells (FIG. 1B). Since this transcript was not detected in the absence of cycloheximide—an inhibitor of nonsense-mediated decay—this aberrant transcript is most-likely degraded within the cell. Alternatively, in case the mutant RNA would be translated, the 35-nt extension causes a frame-shift that is predicted to result in premature termination of protein synthesis. We have assessed whether AON administration can restore the splice defect associated with the c.768G>T mutation in ABCA4, by designing AONs that would block the alternative splice donor site in intron 6, thereby aiming to redirect the splicing machinery back to the original splice donor site.

### Example 1

First, in the Materials and Methods section, the experimental details are described, whereas the results are described and illustrated in the Results section further below.

Materials and Methods

AON Design and Testing

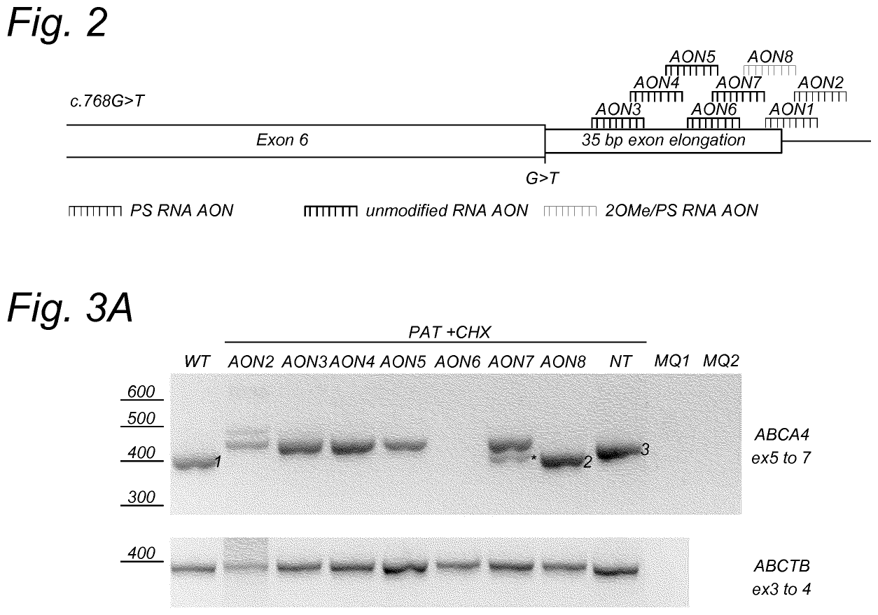

To design the AONs, a smaller region of interest of 257 bp was selected (SEQ ID NO: 5). Subsequently, oligonucleotides of ˜20 nucleotides in length were designed. All oligonucleotides were subjected to in silico RNA structure prediction. Eight AONs that showed differences in predicted structure and/or had the best accessibility were designed and ordered in different chemistries (see Table 2 and FIG. 2).

TABLE 2
AON's

| | SEQ | | |
|---|---|---|---|
| | ID | | Chemical |
| AON# | NO: | AON sequence (5′->3′) | modifications |
| AON1 | 6 | ACCCCAGGAAUCACCUUGCA | 2′O-methyl (2′-OMe) |
| AON2 | 10 | GCUCUGCUACCCCAGGAAUC | 2′-OMe |
| AON3 | 14 | CAAUUGGCGAGCAGCCAAA | unmodified |
| AON4 | 18 | AUCACCUUGCAAUUGGCGAG | unmodified |
| AON5 | 22 | GAGCAGCCAAACCCCUCCCU | unmodified |
| AON6 | 26 | UUGGCGAGCAGCCAAACCCC | unmodified |
| AON7 | 30 | CUUGCAAUUGGCGAGCAGCC | unmodified |
| AON8 | 34 | GGAAUCACCUUGCAAUUGGCG | 2′-OMe and |
| | | | phosphorothioate |
| | | | backbone |

AON Testing

A skin biopsy from an STGD1 patient homozygously carrying the c.768G>T was obtained and a fibroblast cell line was generated. The fibroblast cell line was then transfected with the eight different AONs (described in Table 2) in the in the presence of cycloheximide. The transfected cells were then subjected to RT-PCR analysis.

RT-PCR Analysis

Total RNA was isolated by using the NucleoSpin RNA Clean-up Kit (catalog no., 740955-50; Macherey-Nagel, Düren, Germany) according to the manufacturer's protocol. RNA was quantified and cDNA was synthesized from 1 μg RNA by using the iScript cDNA synthesis kit (catalog no., 1708891; Bio-Rad, Hercules, Calif.) following the manufacturer's instructions. Finally, the efficacy of the AONs was assessed by performing a nested PCR using the following ABCA4 primers: PCR1 (exon4_Fw: 5′-CACCCGGAGAGAATTGCAG-3′ (SEQ ID NO: 38) and exon8_Rv: 5′-CCTGCATACTCGGCCGATG-3′ (SEQ ID NO: 39)); PCR2 or nested PCR (exon5_Fw: 5′-GGAATACGAATAAGGGATATCTTG-3′ (SEQ ID NO: 40) and exon7_Rv: 5′-CTTGAATTCTTGGTGACATATCAG-3′(SEQ ID NO: 41)).

Results

AON8 was able to block the able to block the alternative splice site in intron 6 and redirect the splice site back to the original splice donor site that, albeit is weakened due to the c.768G>T mutation, still can be employed (FIG. 3A and B). As a result, ABCA4 pre-mRNA splicing can be restored.

### Example 2

Here, we report on expansion of example 1, using more AONs (different in terms of sequence as well as chemistry) and employing multiple cellular systems (HEK293T cells and patient-derived fibroblasts.

Materials and Methods

AON Design

To design the AONs, a small ABCA4 region of 235 bp was selected; ABCA4 c.669−c.768+135. Subsequently, a total of 26 AONs (each 21 nucleotides in length) were designed and labelled AON10 to AON35. AON10 to AON34 were designed with 2′-O-Methyl (2′-OMe) chemical modification and AON35 was designed with 2′O-methoxyethyl (2′-O-MOE) chemistry. Following the first results, five more AONs with 2′-O-MOE chemistry were ordered, labelled AON36-AON40 and corresponding to the sequences of AON14, -15, -16, -19 and 23, respectively. For each chemistry, a sense oligonucleotide was ordered, harboring the sequences complementary to AON22 and -35, respectively. All AONs (and SONs) are listed in Table 3. All listed AON's have a phosphorothioate backbone.

TABLE 3
AON's

| | SEQ | | |
|---|---|---|---|
| | ID | | Chemical |
| AON# | NO: | AON sequence (5′->3′) | modification |
| AON10 | 47 | CGAGCAGCCAAACCCCUCCCU | 2′-OMe |
| AON11 | 48 | GGCGAGCAGCCAAACCCCUCC | 2′-OMe |
| AON12 | 49 | UUGGCGAGCAGCCAAACCCCU | 2′-OMe |
| AON13 | 50 | AAUUGGCGAGCAGCCAAACCC | 2′-OMe |
| AON14 | 51 | GCAAUUGGCGAGCAGCCAAAC | 2′-OMe |
| AON15 | 52 | UUGCAAUUGGCGAGCAGCCAA | 2′-OMe |
| AON16 | 53 | CCUUGCAAUUGGCGAGCAGCC | 2′-OMe |
| AON17 | 54 | CACCUUGCAAUUGGCGAGCAG | 2′-OMe |
| AON18 | 55 | UCACCUUGCAAUUGGCGAGCA | 2′-OMe |
| AON19 | 56 | AUCACCUUGCAAUUGGCGAGC | 2′-OMe |
| AON20 | 57 | AAUCACCUUGCAAUUGGCGAG | 2′-OMe |
| AON21 | 58 | GAAUCACCUUGCAAUUGGCGA | 2′-OMe |
| AON22 | 59 | GGAAUCACCUUGCAAUUGGCG | 2′-OMe |
| AON23 | 60 | AGGAAUCACCUUGCAAUUGGC | 2′-OMe |
| AON24 | 61 | CAGGAAUCACCUUGCAAUUGG | 2′-OMe |
| AON25 | 62 | CCAGGAAUCACCUUGCAAUUG | 2′-OMe |
| AON26 | 63 | CCCAGGAAUCACCUUGCAAUU | 2′-OMe |
| AON27 | 64 | CCCCAGGAAUCACCUUGCAAU | 2′-OMe |
| AON28 | 65 | ACCCCAGGAAUCACCUUGCAA | 2′-OMe |
| AON29 | 66 | UACCCCAGGAAUCACCUUGCA | 2′-OMe |
| AON30 | 67 | CUACCCCAGGAAUCACCUUGC | 2′-OMe |
| AON31 | 68 | GCUACCCCAGGAAUCACCUUG | 2′-OMe |
| AON32 | 69 | UGCUACCCCAGGAAUCACCUU | 2′-OMe |
| AON33 | 70 | CUGCUACCCCAGGAAUCACCU | 2′-OMe |
| AON34 | 71 | UCUGCUACCCCAGGAAUCACC | 2′-OMe |
| AON35 | 72 | GGAAUCACCUUGCAAUUGGCG | 2′O-methoxyethyl |
| | | | (2′-O-MOE) |
| AON36 | 73 | GCAAUUGGCGAGCAGCCAAAC | 2′-O-MOE |
| AON37 | 74 | UUGCAAUUGGCGAGCAGCCAA | 2′-O-MOE |
| AON38 | 75 | CCUUGCAAUUGGCGAGCAGCC | 2′-O-MOE |
| AON39 | 76 | AUCACCUUGCAAUUGGCGAGC | 2′-O-MOE |
| AON40 | 77 | AGGAAUCACCUUGCAAUUGGC | 2′-O-MOE |
| 2′-OMe SON | 78 | CGCCAAUUGCAAGGUGAUUCC | 2′-OMe |
| 2′-MOE SON | 79 | CGCCAAUUGCAAGGUGAUUCC | 2′-O-MOE |

AON-Walk In HEK293T Cells

HEK293T Cell Transfection

All tests were performed in duplicates. HEK293T cells were seeded into a 6 well plate and transfected with 6 μg of midigene containing wild-type ABCA4 exon 6 and intron 6 (BA4_WT) and the same midigene but containing the c.768G>T mutation (BA4_MUT). After overnight incubation, HEK293T cells were digested with trypsin and plated in 12-well plates. After the cells became attached, they were transfected with AONs or SON at 1 μM concentration using FuGENE (catalog no. E2311; Promega) and incubated for 48 h. The transfected cells were analyzed at RNA level by RT-PCR.

RT-PCR Analysis

Total RNA was isolated by using NucleoSpin RNA Clean-up Kit (catalog no.740955250; Macherey-Nagel, Düren, Germany) according to the manufacturer's protocol. RNA was quantified and cDNA was synthesized from 1 μg RNA by using SuperScript VILO cDNA synthesis kit (catalog no. 11755050; Invitrogen) following the manufacturer's instructions and was subsequently diluted with H₂O to working concentration of 20 ng/μl. The efficacy of the AONs was assessed by performing a reverse transcriptase PCR using following BA4 midigene specific primers: ABCA4 ex6_Fw: 5′-CTTCAGCCAGAGACGCGGGGC-3′ (SEQ ID NO: 81) and pCI-Neo-Rho ex5_Rev: 5′-AGGTGTAGGGGATGGGAGAC-3′ (SEQ ID NO: 82). RT-PCR using primers targeting RHO exon 5 was used as a control to assess the midigene transfection efficacy (pCI-Neo-Rho ex5_Fw: 5′-ATCTGCTGCGGCAAGAAC-3′ (SEQ ID NO: 83) and pCI-Neo-Rho ex5_Rev: 5′-AGGTGTAGGGGATGGGAGAC-3′(SEQ ID NO: 84)). RT-PCR analysis was performed with 10 μM of each primer, 2 μM of dNTPs, 2.5 mM of MgCl₂, 1 U of Taq polymerase (Roche, Basel, Switzerland), and 40 ng of cDNA in total reaction of 25 μl using the following PCR conditions: 3 min at 94° C., followed by 35 cycles of 30 sec at 94° C., 30 sec at 58° C., and 2 min at 72° C., with the final elongation of 3 min at 72° C. PCR products were resolved on 2.5% agarose gel and selected bands were confirmed with Sanger sequencing. Observed non-specific bands were not considered for analysis. Fiji software was used to perform a semiquantitative analysis of the heteroduplex, mutant and wild type band. Heteroduplex band was distributed equally between the correct and aberrant transcript for graphical representation.

Comparison of AON Efficacy in Fibroblasts

Fibroblast Cell Transfection

Skin biopsies from a healthy individual and from a STGD1 patient homozygous for ABCA4 c.768G>T were obtained and two fibroblasts cell lines were generated. The fibroblast cells were plated in 6-well plates and transfected with AONs at 1 μM concentration using FuGENE (catalog no. E2311; Promega) in the presence of cycloheximide. The transfected cells were analyzed at RNA level by RT-PCR.

RT-PCR Analysis

Total RNA was isolated by using NucleoSpin RNA Clean-up Kit (catalog no. 740955250; Macherey-Nagel, Düren, Germany) according to the manufacturer's protocol. RNA was quantified and cDNA was synthesized from 1 μg RNA by using SuperScript VILO cDNA synthesis kit (catalog no. 11755050; Invitrogen) following the manufacturer's instructions and was subsequently diluted with H₂O to working concentration of 20 ng/μl. The efficacy of the AONs was assessed by performing a RT-PCR on fibroblasts control cell line using following ABCA4 primers: exon5_Fw: 5′ GGAATACGAATAAGGGATATCTTG-3′ (SEQ ID NO: 85) and exon7_Rev: 5′-CTTGAATTCTTGGTGACATATCAG-3′ (SEQ ID NO: 86) and nested PCR on fibroblasts carrying ABCA4 c.768G>T using following ABCA4 primers: PCR1 exon4_Fw: 5′-CACCCGGAGAGAATTGCAG-3′ (SEQ ID NO: 87) and exon8_Rev: 5′-CCTGCATACTCGGCCGATG-3′ (SEQ ID NO: 88) and PCR2 exon5_Fw: 5′ GGAATACGAATAAGGGATATCTTG-3′ (SEQ ID NO: 89) and exon7_Rev: 5′-CTTGAATTCTTGGTGACATATCAG-3′ (SEQ ID NO: 90). The RT-PCR on fibroblasts control cell line was performed with 10 μM of exon5_Fw and exon7_Rw primer, 2 μM of dNTPs, 2.5 mM of MgCl₂, 1 U of Taq polymerase (Roche, Basel, Switzerland), and 80 ng of cDNA in total reaction of 25 μI using the following PCR conditions: 3 min at 94° C., followed by 35 cycles of 30 sec at 94° C., 30 sec at 58° C., and 2 min at 72° C., with the final elongation of 3 min at 72° C. The RT-PCR on fibroblasts carrying ABCA4 c.768G>T mutation was performed with 10 μM of exon4_Fw primer and exon8_Rw primer, 2μM of dNTPs, 2.5 mM of MgCl₂, 1 U of Taq polymerase (Roche, Basel, Switzerland), and 80 ng of cDNA in total reaction of 25 μI using the following PCR conditions: 3 min at 94° C., followed by 40 cycles of 30 sec at 94° C., 30 sec at 58° C., and 2 min at 72° C., with the final elongation of 3 min at 72° C. The nested PCR was performed as described above, 0.5 μl of PCR1 product was used as a PCR template for the nested PCR using the following conditions 3 min at 94° C., followed by 35 cycles of 30 sec at 94° C., 30 sec at 58° C., and 2 min at 72° C., with the final elongation of 3 min at 72° C. All PCR products were resolved on 2.5% agarose gel and selected bands were confirmed with Sanger sequencing. Fiji software was used to perform a semiquantitative analysis of the mutant and wild type band. Actin (ACTB) was amplified to serve as a loading control. Primer: P49750_Fw_ACTB exon 3: 5′-ACTGGGACGACATGGAGAAG-3′ (SEQ ID NO: 91) Primer: P49751_Rev_ACTB exon 4: 5′-TCTCAGCTGTGGTGGTGAAG-3′ (SEQ ID NO: 92).

Results and Discussion

AON Rescue in HEK293T Cells

To identify the most potent AON that is able to rescue the splice defect associated with the c.768G>T mutation (a 35-nt extension), an ‘oligo-walk’ was performed in which AON sequences that target the region around the alternative splice site (at position c.768+35) and only differ one or two nucleotides from one to the next (FIG. 4). Midigenes harboring a genomic fragment of ABCA4 having either a G (WT) or T (MUT) at position c.768 were transfected into HEK293T cells, and subsequently co-transfected without or with the different AONs. In the first series, the majority of AONs contained the previously used chemistry with a phosphorothioate backbone and a 2′-O-Methyl modification (2′-OMe), only AON35 was different and had a 2′-O-methoxyethyl (2′-O-MOE) instead of 2′-OMe, with the same sequence as AON22. As can be observed from FIG. 5, all AONs were capable of to some extent redirecting the splicing defect observed when transfecting the MUT midigene. AONs 14, -15, -16, -19, -22 and -23 appeared to be the most potent when harboring a 2′-OMe chemistry. Intriguingly, AON35, with the recently ‘released’ 2′-O-MOE chemistry, was most capable of redirecting the splicing towards normal ABCA4 transcripts, as confirmed by quantification and Sanger sequencing. All experiments were performed in duplo. Results for AON administration to HEK293T cells with the WT midigene did not show any splicing defects (data not shown). Given the promising results with the 2′-O-MOE chemistry, we decided to order the AONs with the sequence of AONs14, -15, -16, -19 and 23 (AON35 already corresponded to AON22) also with the 2′-O-MOE chemistry. These were coined AONs 36-40, and their potential efficacy was assessed in HEK293T cells in the same manner. As can be observed in FIG. 6, the previously effective AON35, but also AON36, -38 and -40 were highly capable of correcting the aberrant splicing processes. AON39 was a bit less effective while AON37 barely showed rescue.

AON Rescue in Fibroblasts

Next, for the most effective AONs in the HEK293T cells, six for each chemistry, we aimed to assess their splice redirection ability in patient-derived fibroblast cells, as these cells harbor the c.768G>T mutation in their genomic DNA and thus allow the assessment of the associated splice defect with endogenous ABCA4 expression. Fibroblast cells from an individual with STGD1 harboring the c.768G>T mutation in a homozygous state were transfected with the most potent AONs, either having a 2′-OMe or a 2′-O-MOE chemistry. As shown in FIG. 7, some correction of splicing was measured, yet the efficacy was less compared to the results obtained in the HEK293T cells. Of all AONs, AON35 and -40, both having the 2′-O-MOE chemistry appeared to have the strongest effect. The reason for having less efficacy in fibroblasts compared to HEK293T cells is most likely explained by the fact that in HEK293T cells, only a fraction of the ABCA4 transcript was expressed while in the fibroblast, the entire mRNA is there. Thus, the folding of the pre-mRNA molecule can be entirely different, also influencing AON access. In addition, the transfection efficacy can differ per cell as well as the stability of ABCA4 pre-mRNA.

Conclusions

By expanding the number of AONs potentially capable of redirecting aberrant ABCA4 splicing due to the c.768G>T mutation, a number of interesting findings have been made. First, when using the ABCA4 midigene system combined with an ‘oligo-walk’, we discovered that almost all AONs were to some extent able to redirect splicing, yet significant differences between AONs were observed. AON14, -15, -16, -19, -22 and -23, all with the 2′-OMe chemistry, were most efficacious. Intriguingly, AON35, that had a 2′-O-MOE chemistry, outperformed all the 2′-OMe chemistries. Similar observations were made for a number of additional AONs (AON36-AON40), all with the 2′-O-MOE chemistry, yet again with different efficacies between AON sequences. In the fibroblast cells, AONs were not as effective compared to the HEK293T cells, suggesting that either the uptake of the AONs by these cells is less effective, and/or the accessibility of target transcript for the AON is less, due to e.g. folding of ABCA4 pre-mRNA in the endogenous context, something which is not an issue in the HEK293T midigene system.

Taken together, we have identified the most potent AON sequences to target the splice defect caused by c.768G>T, we showed that AONs with the 2′-O-MOE chemistry appear to be more effective compared to those with the 2′-OMe chemistry.

### REFERENCES

Albert S, Garanto A, Sangermano R, Khan M, Bax N M, Hoyng CB, Zernant J, Lee W, Allikmets R, Collin R W J, Cremers F P M (2018). Identification and Rescue of Splice Defects Caused by Two Neighboring Deep-Intronic ABCA4 Mutations Underlying Stargardt Disease. Am J Hum Genet, 102: 517-527.

Allikmets, R., Singh, N., Sun, H., Shroyer, N. F., Hutchinson, A., Chidambaram, A., Gerrard, B., Baird, L., Stauffer, D., Peiffer, A., Rattner, A., Smallwood, P., Li, Y., Anderson, K. L., Lewis, R. A., Nathans, J., Leppert, M., Dean, M. & Lupski, J. R. A photoreceptor cell-specific ATP-binding transporter gene (ABCR) is mutated in recessive Stargardt macular dystrophy. Nat. Genet. 15, 236-246 (1997), doi:10.1038/ng0397-236.

Bauwens, M., De Zaeytijd, J., Weisschuh, N., Kohl, S., Meire, F., Dahan, K., Depasse, F., De Jaegere, S., De Ravel, T., De Rademaeker, M., Loeys, B., Coppieters, F., Leroy, B. P. & De Baere, E. An augmented ABCA4 screen targeting noncoding regions reveals a deep intronic founder variant in Belgian Stargardt patients. Hum. Mutat. 36, 39-42 (2015), doi:10.1002/humu.22716.

Bax, N. M., Sangermano, R., Roosing, S., Thiadens, A. A., Hoefsloot, L. H., van den Born, L. I., Phan, M., Klevering, B. J., Westeneng-van Haaften, C., Braun, T. A., Zonneveld-Vrieling, M. N., de Wijs, I., Mutlu, M., Stone, E. M., den Hollander, A. I., Klaver, C. C., Hoyng, C. B. & Cremers, F. P. M. Heterozygous deep-intronic variants and deletions in ABCA4 in persons with retinal dystrophies and one exonic ABCA4 variant. Hum. Mutat. 36, 43-47 (2015), doi:10.1002/humu.22717.

Braun, T. A., Mullins, R. F., Wagner, A. H., Andorf, J. L., Johnston, R. M., Bakall, B. B., Deluca, A. P., Fishman, G. A., Lam, B. L., Weleber, R. G., Cideciyan, A. V., Jacobson, S. G., Sheffield, V. C., Tucker, B. A. & Stone, E. M. Non-exomic and synonymous variants in ABCA4 are an important cause of Stargardt disease. Hum. Mol. Genet. 22, 5136-5145 (2013), doi:10.1093/hmg/ddt367.

Collin R W J, den Hollander A I, van der Velde-Visser S, Bennicelli J, Bennett J, Cremers F P M (2012). Antisense oligonucleotide (AON)-based therapy for Leber Congenital Amaurosis caused by a frequent mutation in CEP290. Mol Ther Nucl Acids, e14.

Cremers, F. P. M., van de Pol, D. J., van Driel, M., den Hollander, A. I., van Haren, F. J., Knoers, N. V., Tijmes, N., Bergen, A. A., Rohrschneider, K., Blankenagel, A., Pinckers, A. J., Deutman, A. F. & Hoyng, C. B. Autosomal recessive retinitis pigmentosa and cone-rod dystrophy caused by splice site mutations in the Stargardt's disease gene ABCR. Hum. Mol. Gen. 7, 355-362 (1998).

Fujinami, K., Zernant, J., Chana, R. K., Wright, G. A., Tsunoda, K., Ozawa, Y., Tsubota, K., Webster, A. R., Moore, A. T., Allikmets, R. & Michaelides, M. ABCA4 gene screening by next-generation sequencing in a British cohort. Invest. Ophthalmol. Vis. Sci. 54, 6662-6674 (2013), doi:10.1167/iovs.13-12570.

Garanto A, Chung D C, Duijkers L, Corral-Serrano J C, Messchaert M, Xiao R, Bennett J, Vandenberghe L H, Collin R W J (2016). In vitro and in vivo rescue of aberrant splicing in CEP290-associated LCA by antisense oligonucleotide delivery. Hum Mol Genet, 25: 2552-2563.

Garanto A & Collin R W J (2018). Design and in vitro use of antisense oligonucleotides to correct pre-mRNA splicing defects in inherited retinal dystrophies. Methods Mol Biol, 1715: 61-78.

Garanto, A., et al., In vitro and in vivo rescue of aberrant splicing in CEP290-associated LCA by antisense oligonucleotide delivery. Hum Mol Genet, 2016. 25(12): p. 2552-2563.

Lewis, R. A., Shroyer, N. F., Singh, N., Allikmets, R., Hutchinson, A., Li, Y., Lupski, J. R., Leppert, M. & Dean, M. Genotype/Phenotype analysis of a photoreceptor-specific ATP-binding cassette transporter gene, ABCR, in Stargardt disease. Am. J. Hum. Genet. 64, 422-434 (1999), doi:10.1086/302251.

Maugeri, A., van Driel, M. A., van de Pol, D. J., Klevering, B. J., van Haren, F. J., Tijmes, N., Bergen, A. A., Rohrschneider, K., Blankenagel, A., Pinckers, A. J., Dahl, N., Brunner, H. G., Deutman, A. F., Hoyng, C. B. & Cremers, F. P. M. The 2588G-->C mutation in the ABCR gene is a mild frequent founder mutation in the Western European population and allows the classification of ABCR mutations in patients with Stargardt disease. Am. J. Hum. Genet. 64, 1024-1035 (1999).

Maugeri, A., Klevering, B. J., Rohrschneider, K., Blankenagel, A., Brunner, H. G., Deutman, A. F., Hoyng, C. B. & Cremers, F. P. M. Mutations in the ABCA4 (ABCR) gene are the major cause of autosomal recessive cone-rod dystrophy. Am. J. Hum. Genet. 67, 960-966 (2000), doi:10.1086/303079.

Martinez-Mir, A., Paloma, E., Allikmets, R., Ayuso, C., del Rio, T., Dean, M., Vilageliu, L., Gonzalez-Duarte, R. & Balcells, S. Retinitis pigmentosa caused by a homozygous mutation in the Stargardt disease gene ABCR. Nat. Genet. 18, 11-12 (1998), doi:10.1038/ng0198-11.

Rivera, A., White, K., Stohr, H., Steiner, K., Hemmrich, N., Grimm, T., Jurklies, B., Lorenz, B., Scholl, H. P., Apfelstedt-Sylla, E. & Weber, B. H. A comprehensive survey of sequence variation in the ABCA4 (ABCR) gene in Stargardt disease and age-related macular degeneration. Am. J. Hum. Genet. 67, 800-813 (2000), doi:10.1086/303090.

Sangermano R, Bax N M, Bauwens M, van den Born L I, de Baere E, Garanto A, Collin R W J, Goercharn-Ramlal A S, den Engelsman-van Dijk A H, Rohrschneide K, Hoyng C B, Cremers F P M, Albert S (2016). Photoreceptor progenitor mRNA analysis reveals exon skipping resulting from the ABCA4 c.5461-10T>C mutation in Stargardt disease. Ophthalmology, 123: 1375-1385.

Sangermano R, Khan M, Cornelis S S, Richelle V, Albert S, Garanto A, Elmelik D, Qamar R, Lugtenberg D, van den Born L I, Collin R W J, Cremers F P M (2018). ABCA4 midigenes reveal the full splice spectrum of all reported noncanonical splice site variants in Stargardt disease. Genome Res, 28: 100-110.

Schulz, H. L., Grassmann, F., Kellner, U., Spital, G., Ruther, K., Jagle, H., Hufendiek, K., Rating, P., Huchzermeyer, C., Baier, M. J., Weber, B. H. & Stohr, H. Mutation spectrum of the ABCA4 gene in 335 Stargardt disease patients from a multicenter German cohort-impact of selected deep intronic variants and common SNPs. Invest. Ophthalmol. Vis. Sci. 58, 394-403 (2017), doi:10.1167/iovs.16-19936.

van Driel, M. A., Maugeri, A., Klevering, B. J., Hoyng, C. B. & Cremers, F. P. M. ABCR unites what ophthalmologists divide(s). Ophthalmic Genet. 19, 117-122 (1998).

Webster, A. R., Heon, E., Lotery, A. J., Vandenburgh, K., Casavant, T. L., Oh, K. T., Beck, G., Fishman, G. A., Lam, B. L., Levin, A., Heckenlively, J. R., Jacobson, S. G., Weleber, R. G., Sheffield, V. C. & Stone, E. M. An analysis of allelic variation in the ABCA4 gene. Invest. Ophthal. Vis. Sci. 42, 1179-1189 (2001).

Zernant, J., Lee, W., Collison, F. T., Fishman, G. A., Sergeev, Y. V., Schuerch, K., Sparrow, J. R., Tsang, S. H. & Allikmets, R. Frequent hypomorphic alleles account for a significant fraction of ABCA4 disease and distinguish it from age-related macular degeneration. J. Med. Genet. 54, 404-412 (2017), doi:10.1136/jmedgenet-2017-104540.

Zernant, J., Schubert, C., Im, K. M., Burke, T., Brown, C. M., Fishman, G. A., Tsang, S. H., Gouras, P., Dean, M. & Allikmets, R. Analysis of the ABCA4 gene by next-generation sequencing. Invest. Ophthalmol. Vis. Sci. 52, 8479-8487 (2011), doi:10.1167/iovs.11-8182.

Zhou, S., et al., Differentiation of human embryonic stem cells into cone photoreceptors through simultaneous inhibition of BMP, TGFbeta and Wnt signaling. Development, 2015. 142(19): p. 3294-306.

1. An antisense oligonucleotide for redirecting splicing that binds to and/or is complementary to a polynucleotide with the nucleotide sequence as shown in SEQ ID NO: 4, preferably the antisense oligonucleotide binds to or is complementary to a polynucleotide with SEQ ID NO: 5, more preferably the antisense oligonucleotide binds to or is complementary to a polynucleotide with SEQ ID NO: 80, even more preferably the antisense oligonucleotide binds to or is complementary to a polynucleotide selected from the group consisting of SEQ ID NO: 7, 8, 9; 11, 12,13; 15,16,17; 19, 20, and 21.

2. An antisense oligonucleotide for redirecting splicing according to claim 1, wherein a nucleotide in the antisense oligonucleotide may be an RNA residue, a DNA residue, or a nucleotide analogue or equivalent.

3. An antisense oligonucleotide for redirecting splicing according to claim 1, wherein the antisense oligonucleotide has a length of from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides.

4. An antisense oligonucleotide for redirecting splicing according to claim 1, wherein the antisense oligonucleotide comprises or consists of SEQ ID NO: 34, 51, 52, 53, 54, 55, 56, 57, 58, 59, 60, 61, 73, 75 or 77.

5. An antisense oligonucleotide for redirecting splicing according to claim 1, comprising a 2′-O alkyl phosphorothioate antisense oligonucleotide, such as 2′-O-methyl modified ribose (RNA), 2′-O-ethyl modified ribose, 2′-O-methoxyethyl modified ribose, 2′-O-propyl modified ribose, and/or substituted derivatives of these modifications such as halogenated derivatives.

6. An antisense oligonucleotide for redirecting splicing according to claim 1, wherein the antisense oligonucleotide comprises or consists of SEQ ID NO: 34, 51, 52, 53, 54, 55, 56, 57, 58, 59, 60, 61, 73, 75 or 77, and wherein the antisense oligonucleotide comprises a 2′-O-methyl modified ribose (RNA) or a 2′-O-methoxyethyl modified ribose and a phosphorothioate backbone.

7. A viral vector expressing antisense oligonucleotide for redirecting splicing as defined claim 1 when placed under conditions conducive to expression of the molecule.

8. A pharmaceutical composition comprising an antisense oligonucleotide for redirecting splicing according to claim 1 and a pharmaceutically acceptable excipient.

9. A pharmaceutical composition according to claim 8, wherein the pharmaceutical composition is for intravitreal administration and is dosed in an amount ranged from 0.05 mg and 5 mg of total antisense oligonucleotides.

10. A pharmaceutical composition according to claims 8, wherein the pharmaceutical composition is for intravitreal administration and is dosed in an amount ranged from 0.1 and 1 mg of total antisense oligonucleotides for redirecting splicing per eye, such as about 0.1, 0.2, 0.3, 0.4, 0.5, 0.6, 0.7, 0.8, 0.9 or 1.0 mg of total antisense oligonucleotides for redirecting splicing per eye.

11-14. (canceled)

15. A method for the treatment of an ABCA4-related disease or condition requiring modulating splicing of ABCA4 of individual in need thereof, the method comprising contacting a cell of the individual with an antisense oligonucleotide for redirecting splicing as defined in claim 1.

16. The method according to claim 15, wherein the ABCA4-related disease or condition is Stargardt disease.
